(19) United States
(12) Patent Application Publication
(10) Pub. No.: US 2026/0218150 A1
(43) Pub. Date: Jul. 30, 2026

(54) METHODS AND SYSTEMS FOR ENGINEERING CELLS AND FOR TARGET VALIDATION

(71) Applicant: BRISTOL-MYERS SQUIBB COMPANY, Princeton, NJ (US)

(72) Inventors: Eugene DROKHLYANSKY, Westwood, MA (US); Chao DAI, Auburndale, MA (US); Brian Anthony RABE, Waltham, MA (US); Anna LYUBETSKAYA, Boston, MA (US)

(73) Assignee: BRISTOL-MYERS SQUIBB COMPANY, Princeton, NJ (US)

(21) Appl. No.: 19/145,669

(22) PCT Filed: Jan. 4, 2024
(86) PCT No.: PCT/US2024/010376

§ 371 Date: Jul. 3, 2025

Related U.S. Application Data
(60) Provisional application No. 63/478,880, filed on Jan. 6, 2023.
(60) Provisional application No. 63/589,901, filed on Oct. 12, 2023.

(51) Int. Cl. C12N 9/22; A01K 67/0271; C12N 5/00; C12N 5/09; C12N 9/02; C12N 15/113; C12N 15/867

(52) U.S. Cl. CPC C12N 9/226; A01K 67/0271; C12N 5/0081; C12N 5/0693; C12N 9/0069; C12N 15/113; C12N 15/867; C12Y 113/12007; A01K 2227/105; C12N 2310/20; C12N 2510/00; C12N 2740/15043; C12N 2830/34

(57) ABSTRACT

Provided are vectors, systems, and methods for gene editing that result in reduced immunogenicity in a host.

### CROSS REFERENCE

The application claims the benefit of priority of U.S. provisional patent application No. 63/478,880, filed on Jan. 6, 2023, and U.S. provisional patent application No. 63/589,901, filed on Oct. 12, 2023, which are herein incorporated by reference in their entirety.

### INCORPORATION BY REFERENCE OF SEQUENCE LISTING

The present application is being filed along with a Sequence Listing in electronic format. The Sequence Listing is provided as a file entitled 217372000340SEQLIST.xml. The contents of the electronic sequence listing (217372000340SEQLIST.xml; Size: 74,817 bytes; and Date of Creation: Dec. 8, 2023) is herein incorporated by reference in its entirety.

### FIELD

The present disclosure relates to the field of genome editing and more specifically improved vectors that delivers exogenous genes into human and other mammalian cells. The methods, systems and kits were developed to genetically modify those cells, then involve removing some of those genes (e.g., selectable markers and detection markers) to reduce immunogenic effects of introducing exogenous genes. The present disclosure also relates to administering edited cells into tolerized subjects (e.g., mice) to further decrease immunogenic effects of the remaining genes.

### BACKGROUND

In vivo genetic screening enabled by genomic editing technology (such as CRISPR-Cas) has the potential to reveal new therapeutic targets and to provide new understanding into biological mechanisms. However, such in vivo screening is challenging due to potential immunogenicity of the genomic editing components themselves or ancillary components of genetic editing systems which are exogenous to the host. Providing cells harboring such genomic editing components into immunocompetent hosts can cause cell or tumor rejection in the host or an immune reaction that may confound data.

In particular, gene editing system components such as Cas and guide RNA are often provided to cells on vectors that contain detection or selection markers that are used to identity successful transformants harboring the editing components. However these detection or selection markers can cause such host immune reactions

Thus, new vectors and systems that allow for in vivo gene editing with reduced immunogenicity are needed.

### BRIEF SUMMARY

The present disclosure provides for vectors, systems, cells, methods, and kits related to delivering exogenous genes into human and other mammalian cells. Importantly, the methods, systems and kits of the present invention permit introduction of exogenous genes with decreased immunogenic effects. Such vectors, systems, cells, methods, and kits are advantageous for generating genome edited cells that do not trigger immune reactions or cell/tumor rejections in immunocompetent hosts.

In some aspects, provided herein is a vector comprising i) a nucleic acid encoding a Cas enzyme and/or or a nucleic acid encoding a guide RNA and ii) a nucleic acid encoding a selectable marker, wherein the nucleic acid encoding the selectable marker is flanked by recognition sites for a site-specific recombinase and wherein the nucleic acid encoding the Cas enzyme or the nucleic acid encoding the guide RNA is located outside of the recognition sites for the site-specific recombinase.

In some embodiments, the vector comprises a nucleic acid encoding a Cas enzyme, wherein the Cas enzyme is a type I Cas enzyme, type II Cas enzyme, type III Cas enzyme, type IV Cas enzyme, or type V Cas enzyme. In some embodiments, the Cas enzyme is a Cas9 enzyme, a Cas10 enzyme, or a Cas12 enzyme. In some embodiments, the vector further comprises a promoter operably linked to the nucleic acid encoding the Cas enzyme and the selectable marker.

In some embodiments, the vector comprises a nucleic acid encoding a guide RNA. In some embodiments, the nucleic acid encoding the guide RNA is operably linked to a polymerase III holoenzyme (PolIII) promoter.

In some embodiments, the selectable marker comprises an antibiotic resistance gene. In some embodiments, the antibiotic resistance gene is a blasticidin S deaminase gene (also referred to as BSD), or puromycin resistance gene (also referred to as PAC).

In some embodiments, the promoter is an inducible promoter. In some embodiments, the promoter is a human or murine promoter. In some embodiments, the promoter is selected form the group consisting of human cytomegalovirus (hCMV), human phosphoglycerate kinase (hPGK), murine phosphoglycerate kinase (mPGK), human elongation factor-1α (hEF1a), and murine elongation factor-1α (mEF1a). In some embodiments, wherein the promoter is hEF1a.

In some embodiments, the vector further comprises nucleic acid encoding a detection marker. In some embodiments, the nucleic acid encoding the detection marker is flanked by recognition sites for the site-specific recombinase. In some embodiments, the nucleic acid encoding the detection marker and the nucleic acid encoding the selection marker are flanked by the same recognition sites for the site-specific recombinase or wherein the nucleic acid encoding the detection marker and the selection marker are flanked by different recognition sites for a site-specific recombinase. In some embodiments, the detection marker is a fluorescent protein, optionally wherein the detection marker is green fluorescent protein (GFP), yellow fluorescent protein (YFP), red fluorescent protein (RFP), enhanced green fluorescent protein (EGFP), or a luciferase.

In some embodiments, the vector comprises a nucleic acid encoding an internal ribosome entry site between the nucleic acid encoding the Cas enzyme and the selectable marker.

In some embodiments, the site specific recombinase is selected from the group consisting of Cre recombinase of the P1 bacteriophage (Cre), a D6 site-specific DNA recombinase (Dre), and a flippase recombination enzyme (Flp).

In some embodiments, the vector is a viral vector. In some embodiments, the vector is an adenoviral or a lentiviral vector.

In certain aspects, provided herein is system comprising a vector as disclosed herein.

In some embodiments, the system comprises a first vector comprising the nucleic acid encoding the Cas enzyme and a first selectable marker and a second vector comprises the nucleic acid encoding the guide RNA and a second selectable marker. In some embodiments, the first selectable marker and the second selectable marker are the different.

In some embodiments, an engineered cell comprises the vector or the system as disclosed herein. In some embodiments, the cell is a human cell or a murine cell. In some embodiments, the cell is a cell from a primary tumor or a tumor cell line.

In certain aspects, provided herein is a method of generating engineered cells comprising a nucleic acid encoding a Cas enzyme comprising selecting a plurality of cells comprising a nucleic acid encoding a Cas enzyme, a nucleic acid encoding a guide RNA, and a nucleic acid encoding a selectable marker, and removing from the selected cells the nucleic acid encoding the selectable, wherein the resulting engineered cells comprise the nucleic acid encoding the Cas enzyme and the nucleic acid encoding the guide RNA.

In certain aspects, provided herein is a method of generating cells comprising a nucleic acid encoding a Cas enzyme comprising removing nucleic acid encoding a selectable marker from a vector within a plurality of cells, wherein the vector comprises a nucleic acid encoding a Cas enzyme, a nucleic acid encoding a guide RNA, and the nucleic acid encoding the selectable marker, wherein the resulting cells comprise engineered cells comprising the nucleic acid encoding the Cas enzyme.

In some embodiments, the nucleic acid encoding the selectable marker is removed by providing a site-specific recombinase to the plurality of cells. In some embodiments, the site-specific recombinase is selected from the group consisting of Cre, Dre, and Flp. In some embodiments, providing the site-specific recombinase to the cell comprises providing protein or mRNA encoding the site-specific recombinase to the cell plurality of cells. In some embodiments, the method further comprises performing in vitro transcription to produce the mRNA.

In some embodiments, the method further comprises detecting removal of the nucleic acid encoding the selectable marker. In some embodiments, detecting removal of the selectable marker comprises assaying for nucleic acid encoding the selectable marker.

In some embodiments, prior to selecting or removing, the method comprises transducing or transfecting the cell plurality of cells with one or more vectors comprising the nucleic acid encoding the Cas enzyme, the nucleic acid encoding the guide RNA, and the nucleic acid encoding the selectable marker.

In some embodiments, the method comprises providing two or more vectors to the cell, wherein at least one vector comprises the nucleic acid encoding the Cas enzyme, at least one vector comprises the nucleic acid encoding the guide RNA, and at least one vector comprises the nucleic acid encoding the selectable marker. In some embodiments, the two or more vectors comprises a selectable marker. In some embodiments, each of the selectable markers is flanked by recognition sites for the site-specific recombinase. In some embodiments, the selectable marker comprises two or more distinct selection markers. In some embodiments, the nucleic acid encoding the Cas enzyme and the nucleic acid encoding the guide RNA are provided on two different vectors. In some embodiments, the nucleic acid encoding the Cas enzyme and the nucleic acid encoding the guide RNA are provided on two or more different vectors.

In some embodiments, the method comprises providing first and second vectors, wherein the first vector comprises the nucleic acid encoding the Cas enzyme and nucleic acid encoding a first selectable marker flanked by recognition sites for a site specific-recombinase and wherein the second vector comprises the nucleic acid encoding the guide RNA and nucleic acid encoding a second selectable marker flanked by recognition sites for a site-specific recombinase.

In some embodiments, the nucleic acid encoding the guide RNA and the nucleic acid encoding the Cas enzyme are each operably linked to a promoter.

In some embodiments, the selectable markers are removed following selecting the cells for the selectable marker. In some embodiments, the method further comprises selecting the cells for the selectable marker. In some embodiments, selecting the cells for the selectable marker comprises culturing the cell with an antibiotic. In some embodiments, the method further comprises culturing the cell following selection with the selectable marker.

In some embodiments, the Cas enzyme is Cas9. In some embodiments, the method further comprises assaying the Cas enzyme activity in the engineered cells.

In some embodiments, the nucleic acid encoding the guide RNA is not removed.

In some embodiments, the one or more vectors comprises nucleic acid encoding a detection marker.

In some embodiments, the nucleic acid encoding the detection marker is flanked by recognition sites for the site-specific recombinase. In some embodiments, the nucleic acid encoding the detection marker is flanked by two or more recognition sites for the site-specific recombinase. In some embodiments, the detection marker is a fluorescent protein. In some embodiments, the detection marker is GFP, YFP, RFP, EGFP, or a luciferase. In some embodiments, the method further comprises detecting the detection marker.

In some embodiments, the engineered cell is a primary tumor cell or a tumor cell line. In some embodiments, the engineered cell is a murine cell or a human cell.

In some embodiments, the nucleic acid encoding the guide RNA is operably linked to a PolIII promoter. In some embodiments, the promoter is an inducible promoter. In some embodiments, the nucleic acid encoding the Cas enzyme is operably linked to an inducible promoter. In some embodiments, the promoter is a human promoter or a murine promoter.

In some embodiments, the method comprises selecting cells which comprise a vector as disclosed herein.

In some embodiments, the method further comprises introducing the engineered cells into an organism, a tissue, or an organ. In some embodiments, the organism is tolerized to the Cas enzyme and/or GFP. In some embodiments, the engineered cells are engineered inducible cells that can be induced to express the Cas enzyme after introduction into an organism.

In certain aspects, provided herein are a method of introducing engineered cells comprising a Cas enzyme and a guide RNA into an organism that has an immune response, comprising generating the engineered cells by providing one or more vectors comprising the Cas enzyme, the guide RNA, and a selectable marker to a plurality of cells, culturing the cells such that the plurality of cells express the selectable marker, isolating from the cultured cells, selected cells the express the selectable marker, removing the selectable marker from the isolated, selected cells to produce engineered cells, providing the engineered cells to the organism, wherein the organism is tolerized to the Cas enzyme.

In some embodiments, the method further comprises tolerizing the organism to the Cas enzyme. In some embodiments, tolerizing the organism to the Cas enzyme comprises expressing the Cas enzyme in the organism prior to providing the engineered cells to the organism.

In some embodiments, the engineered cell is implanted or injected into the organism. In some embodiments, the method further comprises culturing the engineered cells prior to providing the engineered cells to the organism.

In some embodiments, the expression of Cas enzyme and/or the guide RNA are operably linked to an inducible promoter. In some embodiments, the method further comprises activating the inducible promoter in the organism.

In some embodiments, expression of the nucleic acid encoding the Cas enzyme and the nucleic acid encoding the guide RNA results in editing in the genome of the engineered cell.

In some embodiments, the organism is a non-human mammal. In some embodiments, the organism is a rat or a mouse. In some embodiments, the organism is the same species as the engineered cell. In some embodiments, the organism is a different species from the cell.

In some embodiments, the Cas enzyme is a Cas9 enzyme.

In some embodiments, the engineered cells are a plurality of mammalian cells. In some embodiments, the mammalian cells are murine cells or human cells. In some embodiments, the engineered cells do not express a selectable marker. In some embodiments, the method comprises applying a selection for engineered cells which comprise a vector as described herein.

In some embodiments, the rate of rejection of the engineered cells by the organism is reduced compared to rate of rejection of the engineered cells introduced into an organism which is not tolerized. In some embodiments, the rejection rate of the engineered cells by the organism is reduced by at least 20% compared to rate of rejection of the engineered cells introduced into an organism which is not tolerized. In some embodiments, the rejection rate of the engineered cells by the organism is reduced by at least 50% to rate of rejection of the engineered cells introduced into an organism which is not tolerized. In some embodiments, the rejection rate of the engineered cells by the organism is reduced by 20%-50% compared to rate of rejection of the engineered cells introduced into an organism which is not tolerized. For example, the rejection rate of the engineered cells by the organism is reduced by at least 25%, 30%, 40%, or 45% compared to rate of rejection of the engineered cells introduced into an organism which is not tolerized. In some embodiments, the engineered cells are tumor cells and the rate of tumor growth in the organism is increased compared to the rate of tumor growth of the engineered cells into an organism which is not tolerized.

In some embodiments, provided herein is a kit comprising the vector, the system or the engineered cell as disclosed herein. In some embodiments, the kit comprises a non-human mammal. In some embodiments, the kit comprises instructions for use according to any of the methods provided herein.

### BRIEF DESCRIPTION OF THE DRAWINGS

FIG. 1A is a drawing of a gene editing system. A Cas9 gene editing protein is introduced into cells by lentiviral transduction. Positive transductants were identified by tracking the detection marker, an enhanced green fluorescent protein (EGFP) signal. The cells are transduced again with a guide RNA (gRNA) construct and selected using a selectable marker for positive transduction events. Cells are then transfected with Cre mRNA to drive bacteriophage P1-encoded recombinase Cre expression. Cre expression removes selectable markers from both constructs. The resulting EGFP+ cells are then introduced into a tolerized organism (e.g., Cas9⁺/EGFP⁺ mice).

FIG. 1B is a schematic showing a Cas9 construct of the gene editing system before and after Cre-mediated selection gene removal. A promoter is selected and cloned into the multiple cloning site upstream of a Cas9. The construct also carries a selection marker encoding blasticidin S deaminase gene (BSD) that is flanked by locus of X(cross)-over in P1(loxP) sites. The selectable marker is removed upon expression of Cre from transfected RNA.

FIG. 1C is a drawing/schematic of a base guide vector construct including a polII bicistronic transcript cassette coding EGFP followed by a lox2772-flanked internal ribosome entry site (IRES)-puromycin acetyltransferase (PAC), which is a puromycin resistance gene, followed, in its 3′ untranslated region (UTR), by the empty guide expression cassette (U6-promoter driven, with the 10× feature capture sequence 1 in the hairpin position). The U6-driven guide cassette allows guide RNA expression using an RNA polymerase III (polIII) promoter and is copied into the 5′ LTR during reverse translation and integration of the viral genome. The selection marker is removed upon expression of Cre from transfected RNA. The EGFP gene is included for monitoring transduction and selection.

FIG. 2A and FIG. 2B are a set of graphs that show EGFP fluorescence pre-guide (FIG. 2A) and post-guide construct transduction and puromycin selection (FIG. 2B). The fluorescence activated cell sorting (FACS) plots show the induction of EGFP fluorescence upon guide construct transduction and puromycin selection in murine colon adenocarcinoma MC38 cells treated with the Cas9 editing system.

FIG. 3A is a graph that quantifies macrophage migration inhibitory factor (MIF) knockdown in MC38 cells and murine melanoma B16F10 cells using a Cas9 editing system. MIF relative quantity was determined by using the negative control single guide RNA (sgRNA) cell line as the baseline control.

FIG. 3B is a graph of MIF protein levels after MIF knockdown in MC38 cells. sgMIF3 and sgMIF4 is able to knock down MIF by at least five-fold compared to the negative control (sgNEG cell line).

FIG. 3C is a graph of MIF protein levels after MIF knockdown in B16F10 cells treated with the Cas9 editing system.

FIG. 4A is a schematic that shows the protocol for mRNA preparation and synthesis, including template preparation, in vitro transcription, and in vitro capping.

FIG. 4B is a group of FACS plots for MC38 cells transduced with or without a guide vector (sgNeg1), and 24 hours post transfection with either Cre mRNA or mCherry mRNA. The FACs plot shows data for cells isolated via FACS and contacted with guide⁺/mRNA⁺; or contacted EGFP⁺/mCherry⁺), as well as data for cells contacted with controls (Guide+ only, mRNA+ only, and neither).

FIG. 5 is a graph that shows quantification of the selection gene pre-removal and post-removal Selection gene relative quantities (RQ), calculated using the Delta-Delta-Ct (ddCT) algorithm approximation method, and normalized to the pre-removal levels.

FIG. 6A is a schematic that shows the experimental setup for the tolerization study. In one group, parental MC38 cells were introduced into a syngeneic mouse line (Group A). The other two groups involved Cas9 engineered MC38 hEF1a cells being introduced into either Cas9-EGFP (tolerized) mice (Group B) or wild-type (WT) mice (Group C).

FIG. 6B is a graph with corresponding legend that shows the tumor rejection rate for each group described in FIG. 6A, the rate of tumor rejection in each group, and corresponding pairwise comparisons. * indicates p<0.05; ** indicates p<0.01.

FIG. 6C is a graph that shows the tumor volume of the individual tumors for mice in Groups A, B and C described in FIG. 6A. Each line represents an individual mouse.

FIG. 6D is a group of photographs and images that show dissected tumors from mice as treated in Group A and Group B described in FIG. 6A. As described above, a mouse in group A was injected with parental MC38 cells. An image was taken of a tumor from the Group A mouse shows that the tumor was EGFP negative. An image was also taken of the tumor removed from a mouse in Group B that was injected with edited cells transduced with guide vector (sgNeg); the resulting tumor from the Group B mouse was EGFP positive.

FIG. 7A is a schematic that shows the experimental setup for the αPD1 response experiment. In one group, parental MC38 cells (top) or Cas9 engineered MC38 hEF1a cells (bottom) were introduced into a Cas9-EGFP (tolerized) mouse line and either treated with an OPD1 antibody or a control IgG antibody.

FIG. 7B is a graph that shows the tumor growth inhibition of tumors grown from either parental MC38 cells or CAS9 engineered MC38 hEF1a cells following treatment with αPD1 antibody.

FIG. 8A is a schematic that shows the experimental setup for a CRISPR screen experiment. KP2 cells were transduced with a viral library encoding guides. The selection markers are then removed, and the cells are cultured, then implanted into tolerized mice carrying Cas9-EGFP. Tumors are isolated and sequenced at 21 days and 28 days post injection.

FIG. 8B is a graph that plots the genes targeted in the screen. Triangle and inverted triangles notate control genes that were not predicted to have an effect on viability. Squares indicate essential genes that were predicted to have an effect on viability, such as Rac1. Gray circles indicate experimental genes. The X axis displays the relative in vitro response after 21 days, and the Y axis displays the relative in vivo response after 21 days.

### DETAILED DESCRIPTION

CRISPR/Cas systems create gene edits or deletions in both a fast and reliable way. However, in vivo, these tools elicit an immune response and tumor rejection which limits their utility. Provided herein are vectors and methods for the streamlined approach for antigen removal from the CRISPR/Cas gene editing system, and enabled retention of Cas while achieving tolerization by implanting tumors into Cas expressing mice (tolerized). This advance permits gene editing while simultaneously decreasing or eliminating the immune reaction caused by the selectable marker.

Provided herein are vectors capable of delivering nucleic acids. In some embodiments, the vectors comprise i) a nucleic acid encoding a Cas enzyme or a nucleic acid encoding a guide RNA and ii) a nucleic acid encoding a selectable marker, wherein the nucleic acid encoding the selectable marker is flanked by recognition sites for a site-specific recombinase and wherein the nucleic acid encoding the Cas enzyme or the nucleic acid encoding the guide RNA is located outside of the recognition sites for the site-specific recombinase. This allows retention of the Cas enzyme in the vector and removal of the selectable marker in order to decrease immunogenicity of the construct.

Also provided herein are methods of generating a cell comprising a nucleic acid encoding a Cas enzyme comprising (a) selecting cells comprising one or more vectors comprising a nucleic acid encoding a Cas enzyme, a nucleic acid encoding a guide RNA, and a nucleic acid encoding a selectable marker for the selectable marker, and (b) removing from the selected cells, the nucleic acid encoding the selectable marker from the vector, wherein the nucleic acid encoding the Cas enzyme is maintained.

Also provided herein are methods of generating cells comprising a nucleic acid encoding a Cas enzyme comprising removing the nucleic acid encoding a selectable marker from a vector in a cell, wherein the vector comprises a nucleic acid encoding a Cas enzyme, a nucleic acid encoding a guide RNA, and the nucleic acid encoding the selectable marker, wherein the nucleic acid encoding the Cas enzyme is maintained.

Also provided herein are methods of introducing cells comprising a Cas enzyme and a guide RNA into an organism that has an immune response, comprising (a) generating the cell by providing one or more vectors comprising the Cas enzyme, the guide RNA, and a selectable marker to the cell, (b) expressing the selectable marker, (c) applying a selection for cells that express the selectable marker to produce a selected cell, (d) removing the selectable marker from the cell, and (e) providing the selected cells to the organism, wherein the organism is tolerized to the Cas enzyme.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### Definitions

In order that the present description may be more readily understood, certain terms are first defined. Additional definitions are set forth throughout the detailed description.

As used herein, the singular forms “a,” “an,” and “the” include plural referents unless the context clearly dictates otherwise. The use of “or” or “and” means “and/or” unless stated otherwise. Furthermore, use of the term “including” as well as other forms, such as “include,” “includes,” and “included,” is not limiting.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

It is understood that wherever aspects are described herein with the language “comprising,” otherwise analogous aspects described in terms of “consisting of” and/or “consisting essentially of” are also provided.

The term “vector,” as used herein, is intended to refer to a nucleic acid molecule/construct capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “vector” includes other forms of expression vectors, such as viral vectors (e.g., replication defective retroviruses, adenoviruses and adeno-associated viruses), which serve equivalent functions.

A “polypeptide” refers to a chain comprising at least two consecutively linked amino acid residues, with no upper limit on the length of the chain. One or more amino acid residues in the protein may contain a modification such as, but not limited to, glycosylation, phosphorylation or disulfide bond formation. A “protein” may comprise one or more polypeptides.

As used herein, the recitation of a numerical range for a variable is intended to convey that the invention can be practiced with the variable equal to any of the values within that range. Thus, for a variable that is inherently discrete, the variable can be equal to any integer value within the numerical range, including the end-points of the range. Similarly, for a variable that is inherently continuous, the variable can be equal to any real value within the numerical range, including the end-points of the range. As an example, and without limitation, a variable that is described as having values between 0 and 2 can take the values 0, 1 or 2 if the variable is inherently discrete, and can take the values 0.0, 0.1, 0.01, 0.001, or any other real values 0 and 2 if the variable is inherently continuous.

As used herein, the terms “genetic modification” and “gene editing” are used interchangeably and refer to the modification of a genetic sequence in a chromosome. Gene editing methods may use an endonuclease that is capable of cleaving a target region in a chromosome (e.g., an exon of coding sequence). After cleavage, repair of double-strand breaks by non-homologous end joining in the absence of a template nucleic acid can result in mutations (e.g., insertions, deletions and/or frameshifts) at the target site. Alternatively, in the presence of a donor sequence homologous to sequences flanking the cleavage site, homologous recombination can repair the double-strand breaks with the introduction of an insertion of sequences from the donor sequence (e.g., missense mutations or transgenes). Gene editing methods are generally classified based on the type of endonuclease that is involved in generating double stranded breaks in the target nucleic acid. Examples include, but are not limited to, Clustered Regularly Interspaced Short Palindromic Repeats (CRISPR)/endonuclease systems, transcription activator-like effector-based nuclease (TALEN), zinc finger nucleases (ZFN), homing endonucleases (e.g., ARC homing endonucleases), meganucleases (e.g., mega-TALs), or a combination thereof. Various gene editing systems using meganucleases, including modified meganucleases, have been described in the art; see, e.g., the reviews by Steentoft et al. (2014), Glycobiology 24(8):663-80; Belfort and Bonocora (2014), Methods Mol Biol. 1123:1-26; Hafez and Hausner (2012), Genome 55(8):553-69; and references cited therein. Gene editing methods may also utilize inactive endonucleases fused to epigenetic modifying domains that cause epigenetic modifications to a genetic sequence in a chromosome. The addition and/or removal of epigenetic modifications reconfigures local chromatin structure, with the potential to provoke long-lasting changes in gene transcription.

As used herein, the term “CRISPR” or “CRISPR/Cas system” refers to an endonuclease comprising a Cas enzyme and a guide RNA that directs DNA cleavage by the Cas protein at a recognition site in the genomic DNA recognized by the guide RNA. Thus, the Cas component of a CRISPR/Cas system is an RNA-guided DNA endonuclease. CRISPR biology, as well as Cas endonuclease sequences and structures, are well known to those of skill in the art (see, e.g., “Complete genome sequence of an M1 strain of Streptococcus pyogenes.” Ferretti J. J., et al., Proc. Natl. Acad. Sci. U.S.A. 98:4658-4663(2001); “CRISPR RNA maturation by trans-encoded small RNA and host factor RNase III.” Deltcheva E., et al., Nature 471:602-607 (2011); and “A programmable dual-RNA-guided DNA endonuclease in adaptive bacterial immunity.” Jinek M., et al., Science 337:816-821 (2012), the entire contents of each of which are incorporated herein by reference).

As used herein, the terms “guide RNA,” “single guide RNA” or “sgRNA” may be used interchangeably and refer to an artificial RNA sequence that can be used to guide a Cas protein to a target sequence on a chromosome which shares homology with a portion of the sgRNA. The guide RNA is a specific RNA sequence that recognizes the target DNA region of interest and directs the Cas nuclease there for editing. The gRNA is made up of two parts: crispr RNA (crRNA) which is the portion with sequence complementary to the target DNA, and a tracr RNA, which serves as a binding scaffold for the Cas nuclease.

As used herein, the terms “polynucleotide”, “nucleotide”, “nucleotide sequence”, “nucleic acid” and “oligonucleotide” can be used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, introns, exons, promoters, guide RNA, messenger RNA (mRNA), cDNA, recombinant polynucleotides, plasmids, cassettes, vectors, isolated DNA of any sequence, isolated RNA of any sequence, nucleic acid probes, and primers. A polynucleotide can comprise one or more modified nucleotides, such as the 5′ m7G cap and nucleoside analogs. Modifications to the polynucleotide can be imparted before, during, or after assembly.

As used herein, the terms “DNA regulatory region,” “control elements,” and “regulatory elements,” are used interchangeably and refer to transcriptional and translational control sequences, such as promoters, enhancers, polyadenylation signals, terminators, internal ribosome entry sites, transcription enhancing elements and the like, that provide for and/or regulate transcription of a non-coding sequence (e.g., guide RNA) or a coding sequence (e.g., Cas coding sequence) and/or regulate translation of an encoded polypeptide.

A “promoter” is a nucleotide sequence that directs the transcription of a gene. Typically, a promoter is located in the 5′ non-coding region of a gene, proximal to the transcriptional start site of a gene. If a promoter is an inducible promoter, then the rate of transcription increases in response to an inducing agent. In contrast, the rate of transcription is not regulated by an inducing agent if the promoter is a constitutive promoter. Repressible promoters are also known.

As used herein, the term “multiple cloning site” refer to a cluster of restriction endonuclease recognition sites on a nucleic acid construct (e.g., a viral vector, transfer vector, expression vector, or naked RNA or DNA).

A cell has been “transformed” or “transfected” or “transduced” by exogenous DNA or RNA, e.g., a lentiviral vector, when such DNA or RNA has been introduced inside the cell. The presence of the exogenous DNA or RNA can result in either a permanent or transient genetic change.

As used herein, the term “host cell” refers to a human or other cell (e.g., mammalian cell, including but not limited to non-human primate, rodent (e.g., mouse or rat)), that is transformed, transfected or transduced with one or more of the disclosed vectors.

As used herein, the term “tumor cell” refers to a cell derived from any well-known cancer cell line or a cell derived from a tumor from a patient.

As used herein, the term “target DNA” refers to a DNA polynucleotide that comprises a “target site” or “target sequence.” The terms “target site” or “target sequence” are also used to refer to a nucleic acid sequence present in a target DNA to which a DNA-targeting segment of a guide RNA (e.g., an sgRNA) has complementarity (e.g., a complementary strand of DNA or RNA may be constructed based on nucleobase complementarity).

As used herein, the terms “sequence-specific recombinase” and “site-specific recombinase” refer to enzymes that recognize and bind to a recognition site, then catalyze a process where DNA molecules are rearranged by breaking and rejoining the strands at the recognition site.

As used herein, the terms “recombination site,” “recombination sites for a site-specific recombinase,” “recognition site,” or “recognition site for a site specific recombinase” refers to nucleic acid sites or sequences which are recognized by a sequence- or site-specific recombinase and which become the crossover regions during the site-specific recombination event. Examples of sequence-specific recombinase target sites include, but are not limited to, lox sites, frt sites, attL/attR sites, rox sites and dif sites.

As used herein, the term “lox site” refers to a nucleotide sequence that the Cre recombinase (encoded by the cre gene of bacteriophage P1), can recognize and catalyze a site-specific recombination. A variety of lox sites are known to the art including but not limited to the naturally occurring loxP (the sequence found in the P1 genome), loxB, loxL and loxR (these are found in the E. coli chromosome) as well as a number of mutant or variant lox sites such as loxP511, lox2272, loxA86, loxA117, loxC2, loxP2, loxP3 and loxP23.

### Vectors

The present disclosure provides vectors used in gene editing. In some embodiments, the vectors comprise viral vectors comprising one or more components of a gene editing system (e.g., a Cas enzyme and/or a guide RNA). These viral vectors may be replication deficient viruses are lacking in one or more genes necessary for capsid production and/or genome packaging. However, these vectors may be produced in packaging cell lines which supply the missing functions through use of a packaging plasmid that is present in the packaging cell line. In the present disclosure, the vectors may be capable of integration and, therefore, may include 5′ and 3′ long terminal repeat (LTR) regions. Integrase and reverse transcriptase are encoded by the pol gene. In some embodiments, the viral vector may be an adenovirus vector or a lentiviral vector.

Vectors may also include other modifications necessary or useful for cloning, replication, expression, selection, or detection. For example, one or more multiple cloning sites (MCS) may be included for inserting different promoter elements, and selectable or detection marker genes can be included to identify successfully transformed cells. Selectable markers may be included to select engineered cells comprising one or more vectors of the present disclosure. In some embodiments, the selectable marker comprises an antibiotic resistance marker. In some embodiments, the antibiotic resistance marker is BSD or PAC. The antibiotic resistance marker encodes for a gene product or protein that confers resistance to an antibiotic. In some embodiments, the antibiotic is blasticidin or puromycin. In some embodiments, the vector comprises i) a nucleic acid encoding a Cas enzyme and/or or a nucleic acid encoding a guide RNA and ii) a nucleic acid encoding a selectable marker. A vector comprising the nucleic acid encoding a selectable marker would be easily identified compared to a vector comprising a nucleic acid not encoding a selectable marker.

In some embodiments, the vectors comprise i) a nucleic acid encoding a Cas enzyme and/or a nucleic acid encoding a guide RNA and ii) a nucleic acid encoding a selectable marker, wherein the nucleic acid encoding the selectable marker is flanked by recombination sites for a site-specific recombinase and wherein the nucleic acid encoding the Cas enzyme or the nucleic acid encoding the guide RNA is located outside of the sites for the site-specific recombinase.

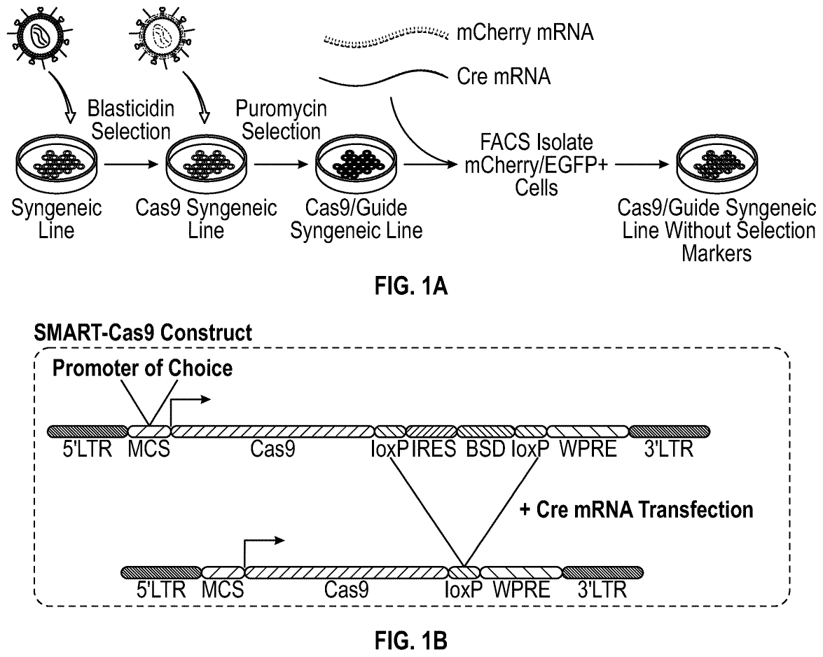

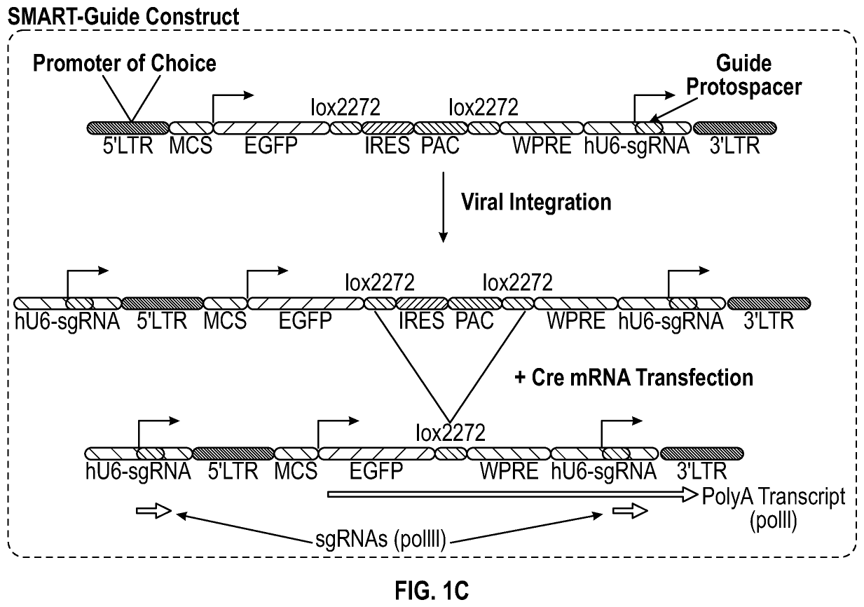

In some embodiments, the vectors comprise i) a nucleic acid encoding a Cas enzyme and ii) a nucleic acid encoding a selectable marker, wherein the nucleic acid encoding the selectable marker is flanked by recombination sites for a site-specific recombinase and wherein the nucleic acid encoding the Cas is located outside of the sites for the site-specific recombinase. In some embodiments, the Cas enzyme is a type I, type II, type III, type IV, or type V Cas enzyme. In some embodiments, the Cas enzyme is a Cas9, a Cas10, or a Cas12. In some embodiments, the Cas enzyme is a Cas9. In some embodiments, the selectable marker comprises an antibiotic resistance marker. In some embodiments, the antibiotic resistance marker is BSD or PAC. The antibiotic resistance marker encodes for a gene product or protein that confers resistance to an antibiotic. In some embodiments, the antibiotic is blasticidin or puromycin. In some embodiments, the promoter is an inducible promoter. In some embodiments, the promoter is a human or murine promoter. In some embodiments, the human promoter is selected from the group consisting of hCMV, hPGK, or hEF1a. In some embodiments, the promoter is hEF1a. In some embodiments, the mouse promoter is selected from the group consisting of mPGK or mEF1a. In some embodiments, the vector comprises a nucleic acid encoding an internal ribosome entry site between the nucleic acid encoding the Cas enzyme and the selectable marker. In some embodiments, the IRES is 3′ of the first loxP site and 5′ of the selectable marker. In some embodiments, the IRES is 3′ of the first loxP site and 5′ of the selection marker. In some embodiments, the vector may comprise a Woodchuck hepatitis virus posttranscriptional regulatory element (WPRE) which creates a tertiary structure enhancing expression. In some embodiments, the WPRE is 5′ of the second loxP site and 3′ of the selection marker. In some embodiments, the vectors may comprise nucleic acid constructs or variants thereof as depicted in FIG. 1B of the present application. In some embodiments, the site-specific recombinase is selected from the group consisting of Cre, Dre, and Flp. In some embodiments, the vector is a viral vector. In some embodiments, the vector is an adenoviral vector or a lentiviral vector. In some embodiments, the promoter is operably linked to the nucleic acid encoding the Cas enzyme and the selectable marker.

In some embodiments, the vectors comprise i) a nucleic acid encoding a guide RNA and ii) a nucleic acid encoding a selectable marker, wherein the nucleic acid encoding the selectable marker is flanked by recombination sites for a site-specific recombinase and wherein the nucleic acid encoding the nucleic acid encoding the guide RNA is located outside of the sites for the site-specific recombinase. In some embodiments, the selectable marker comprises an antibiotic resistance marker. In some embodiments, the antibiotic resistance marker is BSD or PAC. The antibiotic resistance marker encodes for a gene product or protein that confers resistance to an antibiotic. In some embodiments, the antibiotic is blasticidin or puromycin. In some embodiments, the nucleic acid encoding the guide RNA is operably linked to a Pol III promoter. In some embodiments, the promoter is an inducible promoter. A detection marker may also be included in vectors that encode guide RNAs for reasons such as tracking nucleic acid retention, assessing Cas activity, and assessing efficiency of gene editing. In some embodiments, the nucleic acid encoding the vector further comprises a detection marker. In some embodiments, the guide RNA targets a detection marker. In some embodiments, the nucleic acid encoding the detection marker is flanked by recognition sites for the site-specific recombinase. In some embodiments, wherein the nucleic acid encoding the detection marker and the nucleic acid encoding the selection marker are flanked by the same recognition sites for the site-specific recombinase. In some embodiments, wherein the nucleic acid encoding the detection marker and the nucleic acid encoding the selectable marker are flanked by the same recognition sites for the site-specific recombinase. In some embodiments, the nucleic acid encoding the detection marker and the selection marker are flanked by different recognition sites for a site-specific recombinase. In some embodiments, the nucleic acid encoding the detection marker and the selectable marker are flanked by different recognition sites for a site-specific recombinase. In some embodiments, the detection marker is a fluorescent protein, optionally wherein the detection marker is GFP, YFP, RFP, EGFP, or a luciferase. In some embodiments, the detection marker is a fluorescent protein, optionally wherein the detection marker is a GFP, a YFP, a RFP, a EGFP, or a luciferase. In some embodiments, the IRES is 3′ of the first loxP site and 5′ of the selection marker. In some embodiments, the IRES is 3′ of the first loxP site and 5′ of the selectable marker. In some embodiments, the vector may comprise a Woodchuck hepatitis virus posttranscriptional regulatory element (WPRE) which creates a tertiary structure enhancing expression. In some embodiments, the WPRE is 5′ of the second loxP site and 3′ of the selection marker. In some embodiments, the WPRE is 5′ of the second loxP site and 3′ of the selectable marker. In some embodiments, the vectors may comprise nucleic acid constructs or variants thereof as depicted in FIG. 1C of the present application. In some embodiments, the site-specific recombinase is selected from the group consisting of Cre, Dre, and Flp. In some embodiments, the vector is a viral vector. In some embodiments, the vector is an adenoviral or lentiviral vector.

In some embodiments, the vectors comprise i) a nucleic acid encoding a Cas enzyme and ii) a nucleic acid encoding a selectable marker, wherein the nucleic acid encoding the selectable marker is flanked by recombination sites for a site-specific recombinase and wherein the nucleic acid encoding the nucleic acid encoding the Cas is located outside of the sites for the site-specific recombinase. In some embodiments, the vectors further comprise a 5′ and 3′ LTR. In some embodiments, the vectors comprise an IRES. In some embodiments, the selection marker is BSD or PAC. In some embodiments, the selectable marker is BSD or PAC. In some embodiments, the vectors comprise a WPRE. In some embodiments, the Cas is a Cas9. In some embodiments, the selection marker is a BSD cassette. In some embodiments, the selectable marker is a BSD cassette.

In some embodiments, the vector comprise from 5′ to 3′, a nucleic acid encoding Cas9, a recombination site for a site-specific recombinase, IRES, an antibiotic resistance (such as BSD or PAC) cassette, and a recombination site for a site-specific recombinase. In some embodiments, the site specific recombinase is selected from the group consisting of Cre, Dre, and Flp.

In some embodiments, the vectors comprise from 5′ to 3′: nucleic acid encoding a Cas9, a loxP recognition site, an IRES, antibiotic resistance (such as BSD or PAC) cassette, a loxP recognition site, and a WPRE. In some embodiments, the vectors comprise from 5′ to 3′: a 5′ LTR, a multiple cloning site, a nucleic acid encoding Cas9, a loxP recognition site, IRES, an antibiotic resistance (such as BSD) cassette, a loxP recognition site, WPRE, and a 3′ LTR. In some embodiments, the promoter is inserted into the multiple cloning site. In some embodiments, the vectors comprise from 5′ to 3′, a 5′ LTR, a promoter, a nucleic acid encoding Cas9, a loxP recognition site, IRES, an antibiotic resistance (such as BSD or PAC) cassette, a loxP recognition site, WPRE, and a 3′ LTR. In some embodiments, the vectors comprise a nucleic acid encoding from 5′ to 3′, a 5′ LTR, a promoter that is operably linked to a Cas9, loxP, IRES, BSD, loxP, WPRE, and a 3′ LTR. In some embodiments, the vectors comprise a nucleic acid encoding from 5′ to 3′, a 5′ LTR, a hEF1-a promoter, a Cas9, a loxP recognition site, IRES, BSD, a loxP recognition site, WPRE, and a 3′ LTR.

In some embodiments, the vectors comprise i) a nucleic acid encoding a guide RNA and ii) a nucleic acid encoding a selectable marker, wherein the nucleic acid encoding the selectable marker is flanked by recombination sites for a site-specific recombinase and wherein the nucleic acid encoding the nucleic acid encoding the guide RNA is located outside of the sites for the site-specific recombinase. In some embodiments, the vectors further comprise a 5′ and 3′ LTR. In some embodiments, the vectors comprise an IRES. In some embodiments, the vectors comprise a WPRE. In some embodiments, the vectors further comprise a detection marker. In some embodiments, the detection marker is an EGFP. In some embodiments, the selection marker is PAC. In some embodiments, the selectable marker is PAC.

In some embodiments the vector comprises from 5′ to 3′ a promoter, a detection marker, a recombination site for a site-specific recombinase, an IRES, and an antibiotic resistance (such as PAC or BSD) cassette, a recombination site for a site-specific recombinase. In some embodiments, the site specific recombinase is selected from the group consisting of Cre, Dre, and Flp.

In some embodiments, the vector comprises from 5′ to 3′: a Pol III promoter, a nucleic acid encoding a guide RNA, a multiple cloning site, an EGFP, a lox2722 recognition site, an IRES, a PAC cassette, a lox2722 recognition site, and a WPRE. In some embodiments, the vector comprises from 5′ to 3′: a Pol III promoter, a nucleic acid encoding a guide RNA, a 5′ LTR, a multiple cloning site, an EGFP, a lox2722 recognition site, an IRES, a PAC cassette, a lox2722 recognition site, a WPRE, a PolIII promoter operably linked a guide RNA, a guide RNA, and a 3′ LTR. In some embodiments, the multiple cloning site comprises a promoter. In some embodiments, the vector comprises from 5′ to 3′: a Pol III promoter that is operably linked to a nucleic acid encoding a guide RNA, 5′ LTR, a multiple cloning site comprising a promoter, an EGFP, a lox2722 recognition site, an IRES, an antibiotic resistance (such as BSD or PAC) cassette, a lox 2722 recognition site, a WPRE, a PolIII promoter, a guide RNA, and a 3′ LTR.

As will be apparent to one of skill in the art, the constructs disclosed in the present application do not represent all possible variations of the vectors of the invention. For example, components depicted in the figures may be in an alternative order, and additional nucleic acid sequences may be included. Additionally, vectors may also comprise sequences derived from the original native vector (e.g., native viral sequences) that are necessary to the function of the vector (e.g., for integration) or that are unnecessary, as well as sequences which are “artifacts” of the process by which the vector was assembled or cloned. U6 is a type of PolIII promoter. Therefore, in the figures the component “U6” can be a U6 promoter, a PolIII promoter, or any other promoter capable of driving expression of the guide RNA in the host cell. In some embodiments, the promoter is a constitutive promoter. In some embodiments, the promoter is an inducible promoter.

### A. Cas

The present disclosure relates to the vectors comprise viral vectors comprising one or more components of a gene editing system (e.g., a Cas enzyme and/or a guide RNA). The gene editing system introduces genetic modification to a population of cells. In some embodiments, the cells are murine cells or human cells. In some embodiments, the cells are from a primary tumor or a tumor cell line. In some embodiments, the genetic modification is a knock-out of an endogenous gene. In other embodiments, the genetic modification is a knock-in of an exogenous gene.

In some embodiments, the vector comprises a promoter operably linked to a first nucleic acid sequence comprising a first promoter operably linked to a Cas protein coding sequence encoding the open reading frame of a Cas enzyme. In some embodiments, the Cas enzyme is integrated into the host cell genome for stable expression.

CRISPRs (Clustered Regularly Inter spaced Short Palindromic Repeats) comprise a family of DNA loci that are usually specific to a particular bacterial species. The CRISPR locus comprises a distinct class of interspersed short sequence repeats (SSRs) that were recognized in E. coli (Ishino et al. (1987), J. Bacteriol., 169:5429-5433; and Nakata et al. (1989), J. Bacteriol., 171:3553-3556), and associated genes. Similar interspersed SSRs have been identified in Haloferax mediterranei, Streptococcus pyogenes, Anabaena, and Mycobacterium tuberculosis (See, Groenen et al. (1993), Mol. Microbiol., 10:1057-1065; Hoe et al. (1999), Emerg. Infect. Dis., 5:254-263; Masepohl et al. (1996), Biochim. Biophys. Acta 1307:26-30; and Mojica et al. (1995), Mol. Microbiol., 17:85-93. The CRISPR loci typically differ from other SSRs by the structure of the repeats, which have been termed short regularly spaced repeats (SRSRs) (Janssen et al. (2002), OMICS J. Integ. Biol. 6:23 33; and Mojica et al. (2000), Mol. Microbiol. 36:244-246).

The repeats are short elements with a substantially constant length (Mojica et al. (2000), supra). Although the repeat sequences are highly conserved between strains, the number of interspersed repeats and the sequences of the spacer regions typically differ from strain to strain (van Embden et al. (2000), J. Bacteriol. 182:2393-2401. CRISPR loci have been identified in more than 40 prokaryotes (see, e.g., Jansen et al. (2002), Mol. Micro biol. 43:1565-1575) including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium, Thermoanaerobacter; Mycoplasma, Fusobacterium, Azarcus, Chromobacterium, Neisseria, Nitrosomonas, Desulfovibrio, Geobacter, Myxococcus, Campylobacter; Wolinella, Acinetobacter, Erwinia, Escherichia, Legionella, Methylococcus, Pasteurella, Photobacterium, Salmonella, Xanthomonas, Yersinia, Treponema, and Thermotoga.

A “CRISPR system” refers collectively to coding sequences and other elements involved in the expression of CRISPR-associated (“Cas”) genes, including sequences encoding a Cas enzyme, a tracr (transactivating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence, or other sequences and transcripts from a CRISPR locus. In some embodiments, one or more elements of a CRISPR system is derived from a type I, type II, or type III CRISPR system. In some embodiments, an element of a CRISPR system is derived from a particular organism comprising an endogenous CRISPR system, such as Streptococcus pyogenes. In general, a CRISPR system is characterized by elements that promote the formation of a CRISPR complex at the site of a target sequence. In the context of formation of a CRISPR complex, “target sequence” refers to a sequence to which a guide RNA sequence is designed to have complementarity, where hybridization between a target sequence and a guide RNA sequence promotes the formation of a CRISPR complex. Full complementarity is not required, provided there is sufficient complementarity to cause hybridization and promote formation of a CRISPR complex. A target sequence can comprise any polynucleotide, such as DNA or RNA polynucleotides.

As used herein, the term “Cas protein” refers to a CRISPR associated protein, or analog or variant thereof, and embraces any naturally occurring Cas from any organism, any naturally-occurring Cas, any Cas homolog, ortholog, or paralog from any organism, and any analog of a Cas enzyme, naturally-occurring or engineered. The term “Cas” is not meant to be limiting and may be referred to as a “Cas or an analog thereof.”

In some embodiments, proteins comprising Cas or fragments thereof are referred to as “Cas analogs.” A Cas analog shares homology to Cas, or a fragment thereof. Cas analogs include functional fragments of Cas. For example, a Cas9 analog is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 analog may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more amino acid changes compared to a wild type Cas9. In some embodiments, the Cas9 analog comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

Non-limiting examples of Cas proteins include S. pyogenes Cas9 (also known as SpCas9, Csn1 and CSX12), Cpf1, Cas9 nickase, nuclease-inactive Cas9 (also known as dead Cas9), S. aureus Cas9 (SaCas9), Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, CSm3, Csm4, Csm5, Csm6, Cmr1, Cimr3, Cimra, CimrS, Cmr6, Csb1, Csb2, Csb3, CSX17, CSX14, CSX10, CSX16, CsaX, CSX3, CSX1, CSX15, Csf1, Csf2, Csf3, Csf4, C2c1, C2c2 (Cas13a), C2c3 (Cas12c), GeoCas9, CjCas9, Cas12a, Cas12b, Cas12g, Cas12h, Cas12i, Cas13b, Cas13c, Cas13d, Cas14, Csn2, Argonaute, evolved Cas9 domains (xCas9) and circularly permuted Cas9 proteins such as CP1012, CP1028, CP1041, CP1249, and CP1300. These enzymes are known in the art and their nucleic acid and amino acid sequences are publicly available; for example, the amino acid sequence of S. pyogenes Cas9 protein can be found in the SwissProt database under accession number Q99ZW2.

Cas orthologs have been described in various species, including, but not limited to, S. pyogenes, S. thermophiles, C. ulcerans, S. diphtheria, S. syrphidicola, P. intermedia, S. taiwanense, S. iniae, B. baltica, P. torquis, S. thermophiles, L. innocua, C. jejuni, G. thermodenitrificans and N. meningitidis. Additional suitable Cas nucleases and sequences will be apparent to those of skill in the art based on this disclosure. In some embodiments the Cas protein is Cas9, and can be Cas9 from S. pyogenes, S. aureus or S. pneumoniae. For example, the Cas9 enzyme is capable of making effective gene editing in a human cell. In some embodiments, the Cas9 enzyme is capable of making effective gene editing in a human cell line.

In some embodiments, the Cas protein directs cleavage of one or both strands at the location of a target sequence, such as within the target sequence and/or within the complement of the target sequence. In some embodiments, the Cas protein directs cleavage of one or both strands within about 1, 2, 3, 4, 5, 6, 7, 8, 9, 10, 15, 20, 25, 50, 100, 200, 500, or more base pairs from the first or last nucleotide of a target sequence. In other embodiments, a nucleotide sequence encodes for a Cas9 analog. A Cas9 analog, as used herein, refers to other natural occurring or engineered Cas9 that is capable of double-strand DNA cleavage at the site targeted by guide RNA. A non-limiting example of a reduced-size Cas9 analog includes Cpf1 and SaCas9. Cpf1, as used herein, refers to a type II CRIPSR enzyme. Cpf1 mediates robust DNA interference with features distinct from Cas9. Cpf1 is a single RNA-guided endonuclease lacking tracrRNA. Cpf1-mediates DNA cleavage creates DSBs with a short 3′ overhang. Cpf1's staggered cleavage pattern opens up the possibility of directional gene transfer, analogous to traditional restriction enzyme cloning, which may increase the efficiency of gene editing Like the Cas9 variants and orthologs described above, Cpf1 also expands the range of sites that can be targeted by CRISPR to AT-rich regions or AT-rich genomes that lack the NGG PAM sites favored by SpCas9. For instance, the Cas9 protein may comprise a S. pyogenes Cas9-NG variant that recognizes an expanded PAM, i.e., most NG PAM sites. This variant is disclosed in Nishimasu et al., Science 361, 1259-1262 (2018), incorporated herein by reference. In other embodiments, the Cas9 protein may comprise a Cas9 analog that has been evolved to recognize an expanded PAM, as reported in Hu et al., Nature, 556(7699):57-63 (2018) and International Application No. PCT/US2019/47996, filed Aug. 23, 2019, each of which is incorporated by reference herein. Exemplary evolved Cas9 variants having expanded PAM specificities include xCas9 (3.6) and xCas9 (3.7).

In some embodiments, the Cas9 analog is SaCas9. An SaCas9, as used herein, refers to a Cas9 protein derived from Staphylococcus aureus. SaCas9 is ~1 kilobase shorter than SpCas9, which renders it more versatile to be packaged into various vector systems (e.g., AAV vectors, lentiviral vectors). Similar to SpCas9, the SaCas9 endonuclease is capable of modifying target genes in mammalian cells in vitro and in mice in vivo. In some embodiments, the Cas protein is codon optimized for expression in particular cells, such as eukaryotic cells. The eukaryotic cells can be those of or derived from a particular organism, such as a mammal, including but not limited to human, non-human primate, mouse, rat, rabbit dog. In some embodiments, the Cas9 protein is an engineered Cas9 that is capable of recognizing non-NGG PAM sequences.

In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell Biol., 2015 Nov. 5; 60(3): 385-397, which is incorporated herein by reference. In some embodiments, a napDNAbp domain may comprise a CasX (now referred to as Cas12e) or CasY (now referred to as Cas12d) domain, which have been described in, for example, Burstein et al., “New CRISPR-Cas systems from uncultivated microbes.” Cell Res. 2017 Feb. 21. Doi: 10.1038/cr.2017.21, and Liu et al., “CasX enzymes comprise a distinct family of RNA-guided genome editors,” Nature. 2019; 566(7743):218-223, each of which is incorporated herein by reference. In other embodiments, the Cas protein provided herein may be a CjCas9, Cas12a, Cas12b, Cas12g, Cas12h, Cas12i, Cas13b, Cas13c, Cas13d, Cas14, Csn2, and GeoCas9. CjCas9 is described and characterized in Kim et al., Nat Commun. 2017; 8:14500 and Dugar et al., Molecular Cell 2018; 69:893-905, incorporated herein by reference. GeoCas9 is described and characterized in Harrington et al. Nat Commun. 2017; 8(1):1424 and International Publication No. PCT/US2019/58678, filed Oct. 29, 2019, each of incorporated herein by reference. The Cas12a, Cas12b, Cas12g, Cas12 h and Cas12i proteins are described and characterized in, e.g., Yan et al., Science, 2019; 363(6422): 88-91, Murugan et al. The Revolution Continues: Newly Discovered Systems Expand the CRISPR-Cas Toolkit, Molecular Cell 2017; 68(1):15-25, each of which are incorporated herein by reference. Cas14 is characterized and described in Harrington et al. Science 2018; 362(6416):839-842, incorporated herein by reference. Cas13b, Cas13c and Cas13d are described and characterized in Smargon et al., Molecular Cell 2017, Cox et al., Science 2017, and Yan et al. Molecular Cell 70, 327-339.e5 (2018), each of which are incorporated herein by reference. Csn2 is described and characterized in Koo Y., Jung D. K., and Bae E. PloS One. 2012; 7:e33401, incorporated herein by reference.

In some embodiments, the Cas may be a Cas enzyme from a Type I CRISPR-Cas system. In Type I systems, the CRISPR/Cas system involves Cascade (a multimeric complex) to process the guide RNAs, a Cas3 enzyme, and a guide RNA. In some embodiments, the Cas may be a Cas enzyme from the Type III or Type VI CRISPR-Cas system. In some embodiments, the Cas enzyme may be a Cas enzyme from a Type V CRISPR-Cas system. In some embodiments, the Cas enzyme is a CasX or variant thereof. In some embodiments, the Cas is a type 12a or type 12b Cas. In some embodiments, the Cas is CasX.

In some embodiments, the Cas protein is mutated with respect to a corresponding wild-type enzyme such that the mutated Cas protein lacks the ability to cleave one or both strands of a target polynucleotide containing a target sequence. In particular embodiments, an aspartate-to-alanine substitution (D10A) in the RuvC1 catalytic domain of S. pyogenes Cas9 converts Cas9 from a nuclease that cleaves both strands to a nickase that nicks the targeted strand, or the strand that is complementary to the guide RNA. A histidine-to-alanine substitution (H840A) in the HNH catalytic domain of S. pyogenes Cas9 generates a nick on the strand that is displaced by the guide RNA during strand invasion, also referred to herein as the non-edited strand. The single catalytically active nuclease site of the nCas9 leaves a nick in the non-edited strand, which will direct mismatch repair machinery to read (rather than remove) a mutated sequence in the target gene during repair. Other examples of mutations that render Cas9 a nickase include, without limitation, N854A and N863A in SpCas9, and corresponding mutations in other wild-type Cas9 proteins or analogs thereof. Reference is made to U.S. Pat. No. 8,945,839, which is incorporated herein by reference.

In nature, CRISPR clusters are transcribed and processed into CRISPR RNA (crRNA). In certain types of CRISPR systems (e.g., type II CRISPR systems), correct processing of pre-crRNA may require a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves linear or circular nucleic acid target complementary to the RNA. Specifically, the target strand not complementary to crRNA is first cut endonucleolytically, then trimmed 3′-5′ exonucleolytically. In nature, DNA-binding and cleavage may require protein and both RNAs. However, single guide RNAs (“sgRNA”, or “guide RNA”) can be engineered so as to incorporate embodiments of both the crRNA and tracrRNA into a single RNA species—the guide RNA. See, e.g., Jinek M., et al., Science 337:816-821 (2012), which is incorporated herein by reference.

In general, a guide RNA is any polynucleotide sequence having sufficient complementarity with a target polynucleotide sequence to hybridize with the target sequence and direct sequence-specific binding of a CRISPR complex (e.g., a Cas) to the target sequence. In some embodiments, the degree of complementarity between guide RNA and its corresponding target sequence, when optimally aligned using a suitable alignment algorithm, is about or more than about 50%, 60%, 75%, 80%, 85%, 90%, 95%, 96%, 97%, 97.5%, 98, 99%, or more complementary. Optimal alignment can be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAST, Novoalign (Novocraft Technologies, ELAND (Illumina, San Diego, Calif.), SOAP (available at Soap.genomics.org.cn), and Maq (available at maq.Sourceforge.net).

In some embodiments, the guide sequence of the guide RNA is about or more than about 5, 10, 11, 12, 13, 14, 15, 16, 17, 18, 19, 20, 21, 22, 23, 24, 25, 26, 27, 28, 29, 30, 35, 40, 45, 50, 60, 65, 70, 75, or more nucleotides in length. The guide sequence is, in some embodiments, 20 nucleotides long. See U.S. Publication No. 2015/0166981, published Jun. 18, 2015, which is incorporated by reference herein. In some embodiments, the gRNA comprises a guide sequence of at least 10 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that is complementary to a sequence in a target gene.

The guide sequence of the guide RNA is linked to a TRACR (tracr) mate (also known as a “backbone”) sequence which in turn hybridizes to a tracr sequence. In some embodiments, the guide RNAs for use in accordance with the disclosed methods comprise a backbone structure that is recognized by an S. pyogenes Cas9 protein.

In some embodiments, the guide RNA is delivered into the cells as single stranded RNA. In some embodiments, the guide RNA is delivered into the cells on an expression vector. In some embodiments, the guide RNA is delivered into the cells using the same vector as the Cas. In other embodiments, the guide RNA is delivered into the cells using a different vector from the Cas.

In some embodiments, the vector comprises one or more selection markers. In some embodiments, the vector comprises one or more selectable markers. In some embodiments, vector comprises one or more detection markers. In some embodiments, the vector comprises a detection marker and a selection marker. In some embodiments, the vector comprises a detection marker and a selectable marker.

### B. Promoters

The vectors may comprise promoters. See Goldstein et al., 1995 Biotechnol Annu Rev. 1:105-28, Haberle, V., et al., 2018, Nat Rev Mol Cell Biol 19, 621-637 and Liu, X., et al., 2022 Bioprocess Biosyst Eng 45, 955-967. Promoters include eukaryotic promoters as well as viral promoters that function in eukaryotic host cells, and particularly in human, murine and other mammalian host cells. A promoter is a region of DNA upstream of a gene where relevant proteins (such as RNA polymerase and transcription factors) bind to initiate transcription of that gene. The resulting transcription produces an RNA molecule (such as mRNA). A key step in retrieving the information stored in the complex genomes of eukaryotes involves the identification of transcription units and, more specifically, the recognition of promoter sequences by RNA polymerase. In some embodiments, a promoter is where the polymerase binds. In eukaryotes, the task of recognizing nuclear gene promoters and then transcribing the genes is divided among three highly related enzymes, RNA polymerases I, II, and III. Each of these RNA polymerases is dedicated to the transcription of specific sets of genes, and each depends on accessory factors, the so-called transcription factors, to recognize its cognate promoter sequences. The following eukaryotic promoters listed in Table 1 are non-limiting examples of promoters that may be useful.

TABLE 1
Eukaryotic promoters

| Promoter | Description |
|---|---|
| CMV | Strong mammalian expression promoter from |
| | the human cytomegalovirus |
| EF1a | Strong mammalian expression from human |
| | elongation factor 1 alpha |
| SV40 | Mammalian expression promoter from the |
| | simian vacuolating virus 40 |
| PGK1 | Mammalian promoter from phosphoglycerate |
| (human or | kinase gene. |
| mouse) | |
| UbC | Mammalian promoter from the human |
| | ubiquitin C gene |
| human | Mammalian promoter from beta actin gene |
| beta actin | |
| CAG | Strong hybrid mammalian promoter |
| TRE | Tetracycline response element promoter |
| UAS | Drosophila promoter conaining |
| | Gal4 binding sites |
| Ac5 | Strong insect promoter from Drosophila |
| | Actin 5c gene |
| Polyhedrin | Strong insect promoter from baculovirus |
| CaMKIIa | Ca2+/calmodulin-dependent protein |
| | kinase II promoter |
| GAL1, 10 | Yeast adjacent, divergently transcribed |
| | promoters |
| TEF1 | Yeast transcription elongation factor promoter |
| GDS | Strong yeast expression promoter |
| | from glyceraldehyde 3-phosphage |
| | dehydrogenase |
| ADH1 | Yeast promoter for alcohol dehydrogenase I |
| CaMV35S | Strong plant promoter from the |
| | Cauliflower Mosaic Virus |
| Ubi | Plant promoter from maize ubiquitin gene |
| H1 | From the human polymerase III RNA promoter |
| U6 | From the human U6 small nuclear promoter |

In some embodiments, the vector comprises a promoter. In some embodiments, the vector comprises one or more promoters. In some embodiments, the vector comprises a promoter that is used to express a Cas enzyme. In some embodiments, the promoter is selected from the group consisting of hCMV, hPGK, mPGK, hEF1a, and mEF1a. In some embodiments, the promoter is human EF1a (hEF1a). In some embodiments, the promoter is hEF1a. In some embodiments, the promoter is mouse EF1a (mEF1a). In some embodiments, the promoter is EF1a. In some embodiments, the vector comprises a promoter that is used to express a guide RNA. In some embodiments, the promoter is a PolIII promoter. In some embodiments, the promoter is U6.

Promoters may be operably linked to another element, wherein two (2) or more components (e.g., a promoter and a sequence element, a promoter and a gene) are arranged such that both components function normally and allow the possibility that at least one of the components can mediate a function that is exerted upon at least one of the other components. In some embodiments, the vector comprises a promoter that is operably linked to a nucleic acid sequence encoding for a Cas enzyme. In some embodiments, the Cas enzyme is a Cas9. In some embodiments, the promoter is human EF1a. In some embodiments, the promoter is mouse EF1a. In some embodiments, the vector comprises a promoter that is operably linked to a nucleic acid sequence encoding for a guide RNA. In some embodiments, the promoter is a PolIII promoter. In some embodiments, the promoter is U6 promoter. Operably linking a promoter to a nucleic acid encoding for an RNA product thus places the control of expression of that gene under the operably linked promoter. Therefore, in some embodiments, Cas expression is controlled by the EF1a promoter. In some embodiments, expression of the guide RNA is controlled by a PolIII promoter.

A promoter can be a constitutively active promoter (i.e., a promoter that is constitutively or constantly in an active/“ON” state); an inducible promoter (i.e., a promoter that is active/“ON” or inactive/“OFF” depending upon an external stimulus (e.g., the presence of a particular temperature, compound, or protein); a spatially restricted promoter (e.g., tissue specific promoter, cell type specific promoter, etc.); or temporally restricted promoter (i.e., the promoter is in the “ON” state or “OFF” state during specific stages of embryonic development or during specific stages of a biological process (e.g., hair follicle cycle in mice)). In some embodiments, a constitutive promoter is used to drive or control expression of the Cas and/or the guide RNA. In some embodiments, an inducible promoter is used to drive or control expression of the Cas and/or the guide RNA. In some embodiments, the inducible promoter used to drive or control expression of the Cas and/or the guide RNA, wherein the drive or control is further characterized by controllable regulation, efficient induction, and/or effective expression.

Suitable promoters can be derived from viruses, prokaryotic or eukaryotic organisms, and can be used to drive expression by any RNA polymerase (e.g., pol I, pol II, and pol III). Exemplary promoters include, but are not limited to the SV40 early and late gene promoters, zona pellucida sperm-binding protein 3 (Zp3) promoter mouse mammary tumor virus long terminal repeat (LTR) promoter; mouse metallothionein-1 gene promoter; adenovirus major late promoter (Ad MLP); a herpes simplex virus (HSV) thymidine kinase gene promoter, a cytomegalovirus (CMV) promoter such as the CMV immediate early promoter region (CMVI E), a rous sarcoma virus (RSV) promoter, a human U6 small nuclear promoter (U6) (Miyagishi et al. (2002), Nature Biotechnology 20: 497-500), an enhanced U6 promoter (e.g., Xia et al. (2003), Nucleic Acids Res. 31(7)), a human H1 promoter, a eukaryotic translation elongation factor 1 α (EF1α) promoter, and the like.

In some embodiments, the promoter is a constitutive promoter. Constitutive promoters direct expression that is largely, if not entirely, independent of environmental and developmental factors. As their expression is normally not conditioned by endogenous factors, constitutive promoters are usually active across species and even across kingdoms. Non-limiting examples of constitutive promoters are CMV, EF|α. SV40, PGK1, Ubc, human beta actin, CAG, Ac5, Polyhedrin, TEF1m GDS, CaMV355, Ubi, H1, and U6.

In some embodiments, the promoter is an inducible promoter. Inducible promoters are only active under specific circumstances. Non-limiting examples of factors that can activate an inducible promoter include the presence of certain chemical compounds (i.e., inducers) or the absence of certain chemical compounds (i.e., repressors), temperature, light, etc. Non-limiting examples of inducible promoters are TRE, GAL1.10, AlcR, Hsp-70, Hsp-90, FixK2, T7 RNA polymerase promoter, T3 RNA polymerase promoter, Isopropyl-beta-D-thiogalactopyranoside (IPTG)-regulated promoter, lactose induced promoter, heat shock promoters, tetracycline-regulated promoters, steroid-regulated promoters, metal-regulated promoters, estrogen receptor-regulated promoters, etc.

In some embodiments, the promoter is a tissue-specific promoter. Tissue-specific promoters direct the expression of a gene in a specific tissue or at certain developmental state. A transgene operably linked to a tissue-specific promoter can be expressed in the specific tissue where the promoter is active. Non-limiting examples of tissue specific promoters include B29 promoter for expression of transgenes in B cells; CD14 promoter for expression of a transgene in monocytic cells; desmin promoter for expression of transgene in muscle cells; elastase-1 promoter for expression of transgene in pancreatic cells; endoglin promoter for expression of transgene in endothelial cells, and GFAP promoter for expression of transgene in neuron cells.

A promoter may be described by strength (e.g., weak, medium and strong). This describes the promoter's ability to direct relatively low amounts (weak), relatively moderate amounts (medium) or relatively high amounts (strong) of gene expression. A promoter may be strong in one cell type but weak in another cell type. Non-limiting examples of weak promoters include UBC. Non-limiting examples of medium promoters include EFS. Non-limiting examples of strong promoters include: CMV and EF1a. In some embodiments, the promoter may be a weak promoter. In some embodiments, the promoter may be a medium promoter. In some embodiments, the promoter may be a strong promoter. In some embodiments, the promoter used to express the Cas is different from the promoter used to express the guide RNA. In some embodiments, the promoter used to express the Cas is a different strength than the promoter used to express the guide RNA.

### C. Selectable Markers

Selectable markers permit positive selection (i.e., the cells of interest are not killed). Therefore, as used herein, a selectable marker refers to an exogenous gene introduced into the host cell by vector of the invention that confers a trait suitable for artificial selection. See Methods of Molecular Biology editor John M. Walker, electronic ISSN 1940-6029.

In some embodiments, the selectable marker is on the same nucleic acid comprising a nucleic acid sequence encoding a Cas enzyme. In some embodiments, the selectable marker is cis to a nucleic acid sequence encoding a Cas enzyme. In some embodiments, the selectable marker is downstream of a nucleic acid encoding a Cas enzyme. In some embodiments, the selectable marker is 3′ (e.g., downstream) of a nucleic acid encoding a Cas enzyme. In some embodiments, the selectable marker is a gene that is exogenous to the engineered cell. In some embodiments, the selectable marker is a gene that is derived from a different organism than the engineered cell. In some embodiments, the gene is BSD. In some embodiments, the selectable marker confers resistance to blasticidin. In some embodiments, the selectable marker causes an immune response in the engineered cell.

In some embodiments, the selectable marker is on the same nucleic acid comprising a nucleic acid sequence encoding a guide RNA. In some embodiments, the selectable marker is cis to a nucleic acid sequence encoding a guide RNA. In some embodiments, the selectable marker is downstream of a nucleic acid encoding a Cas enzyme. In some embodiments, the selectable marker is 3′ (e.g., downstream) of a nucleic acid encoding a guide RNA. In some embodiments, the selectable marker is a gene that is exogenous to the engineered cell. In some embodiments, the selectable marker is a gene that is derived from a different organism than the engineered cell. In some embodiments, the gene is puromycin-N-acetyltransferase (PAC). In some embodiments, the selectable marker confers resistance to puromycin. In some embodiments, the selectable marker causes an immune response in the engineered cell.

Selectable markers may be included to select for cells containing genetic modifications. The marker gene itself provides convenient selection for the cellular population expressing another gene that has been cotransfected with the marker. Non-limiting examples for selectable markers include antibiotic resistance genes, cell surface markers and enzymes.

Selective antibiotics for generating stable cell lines or other recombinant cultures are chosen based on the antibiotic resistance gene or the selectable marker. Non-limiting examples of antibiotics that are useful for selection and examples of the respective genes that confer resistance to the corresponding antibiotic are disclosed in Table 2.

TABLE 2
Examples of antibiotics as selection markers

| Antibiotic used for selection | Selectable marker |
|---|---|
| Blasticidin | Blasticidin S deaminase gene (bsd) |
| Geneticin (G-418) | aminoglycoside phosphotransferase |
| | 3′(II) (APH (3′)II) |
| | aminoglycoside phosphotransferase |
| | 3′(I) (PH (3′)I) |
| Hygromycin B | E. coli hygromycin resistance |
| | gene (hyg or hph) |
| Mycophenolic Acid | inosine monophosphate |
| | dehydrogenase (IMPDH) |
| Puromycin | puromyein N-acetyltransferase (pac) |
| Zeocin | Zeocin resistance gene (Sh ble) |

Antibiotics fall into antibiotic classes. An antibiotic class is a grouping of different drugs that have similar chemical and pharmacologic properties. Their chemical structures may look comparable, and genes conferring resistance towards a member of an antibiotic class may confer partial resistance to drugs within the same class. Classes of antibiotics include are nucleosides (such as aminonucleocides peptidyl nucleosides), penicillins, tetracyclines, cephalosporins, quinolones, lincomycins, macrolides, sulfonamides, glycopeptides, aminoglycosides, and carbapenems.

Another example may be the use of exogenous enzymes to permit the metabolism of alternative energy sources. Exogenous enzymes may be coupled with glucose-free media to select only for cells that may use complex energy sources.

In some embodiments, the first vector (or “Cas vector”) and/or second vector (or “guide RNA vector”) further comprises one or more selectable markers.

In some embodiments, the selectable marker is an antibiotic resistance gene. Non-limiting examples of antibiotic resistance genes are the puro gene, bls gene, hygro gene, hph gene, sh ble gene, or neo gene. In some embodiments, the selectable marker is the bls gene, and cells that express the bls gene are resistant to blasticidin. In another embodiment, the selectable marker is the puromycin gene, and cells that express the puromycin gene are resistant to puromycin.

In some embodiments, a first vector comprises the nucleic acid encoding the Cas enzyme and a first selectable marker and a second vector comprises the nucleic acid encoding the guide RNA and a second selectable marker. In some embodiments, the first selectable marker and the second selectable marker are the different.

In some embodiments, the selection markers on the Cas vector and the guide RNA vector are different. In some embodiments, the selection markers on the Cas vector and the guide RNA vector are selected by different means. In some embodiments, the selection markers on the Cas vector and the guide RNA are the same. In some embodiments, the selection markers on the Cas vector and the guide RNA vector are selected by the same means (e.g., resistance).

In some embodiments, the selectable markers on the Cas vector and the guide RNA vector are different. In some embodiments, the selectable markers on the Cas vector and the guide RNA vector are selected by different means. In some embodiments, the selectable markers on the Cas vector and the guide RNA are the same. In some embodiments, the selectable markers on the Cas vector and the guide RNA vector are selected by the same means (e.g., resistance).

### D. Detection Markers

Detection marker genes may be used to detect cells transfected with the vectors provided herein. A detection marker, as used herein, refers to an exogenous gene introduced into the host cell by a vector of the invention that confers a trait suitable for detection. Non-limiting examples for selectable markers include fluorescent proteins, cell surface markers and enzymes. The presence of the detection markers may be by various assays known to one of ordinary skill in the art. Non-limiting examples of detection methods include flow cytometric analysis, western blot, microscopy, magnetic or bead selection and RT-qPCR.

In some embodiments, the detection marker is a fluorescent protein. Non-limiting examples of fluorescent proteins are Green Fluorescent Protein (GFP) or EGFP, Red Fluorescent Protein (RFP), Yellow Fluorescent Protein (YFP), Cyan Fluorescent protein (CFP), Blue Fluorescent Protein (BFP), mCherry, and tdTomato. In some embodiments, the detection marker is a fluorescent protein, optionally wherein the detection marker is GFP, YFP, RFP, EGFP, or a luciferase. In some embodiments, the detection marker is a fluorescent protein, optionally wherein the detection marker is a GFP, a YFP, a RFP, a EGFP, or a luciferase. In some embodiments, the detection marker is a GFP. In some embodiments, the detection marker is an EGFP. In some embodiments, the detection marker is encoded by a nucleic acid, and the nucleic acid encoding the detection marker and the nucleic acid encoding the selection marker are flanked by the same recognition sites for the site-specific recombinase or wherein the nucleic acid encoding the detection marker and the selection marker are flanked by different recognition sites for a site-specific recombinase. In some embodiments, the detection marker is encoded by a nucleic acid, and the nucleic acid encoding the detection marker and the nucleic acid encoding the selectable marker are flanked by the same recognition sites for the site-specific recombinase or wherein the nucleic acid encoding the detection marker and the selectable marker are flanked by different recognition sites for a site-specific recombinase.

In some embodiments, the detection marker is a cell surface marker. The presence of the cell surface marker can be detected by staining the cells with an antibody that is specific to the cell surface marker and that is conjugated with a fluorophore.

In some embodiments, the detection marker is an enzyme. Non-limiting examples of an enzymes useful as detectable markers include luciferase, horseradish peroxidase (HRP) and beta-galactosidase. The expression of these enzyme can be detected by adding the corresponding substrate into the cells and detecting the resulting bioluminescent or chromogenic product.

In some embodiments, the detection markers on the Cas vector and the guide RNA vector are detected by different means. In some embodiments, the detection markers on the Cas vector and the guide RNA vector are selected by the same means.

### E. Other DNA Regulatory Elements

In some embodiments, any of the vectors of the invention can comprise one or more individual restriction endonuclease recognition sequences or one or more multiple cloning sites. These sites can be located upstream and/or downstream of one or more sequence elements of one or more vectors. These sites can also be located 5′ (e.g., upstream) and/or 3′ (e.g., downstream) of one or more sequence elements of one or more vectors.

In some embodiments, any of the vectors of the invention can comprise a internal ribosome entry site (IRES) sequence. IRES sequences are commonly used in molecular biology to recruit ribosomes and allow cap-independent translation, which can link two coding sequences in one bicistronic vector and allow the translation of both proteins. An IRES may also be used to link two coding sequences under the same promoter without generating a protein fusion. In some embodiments, one of the two coding sequences is a selection or detection marker. In some embodiments, one of the two coding sequences is a selectable or detection marker. An IRES thus permits a method of selecting or detecting expression of both proteins under the control of the same promoter by proxy by identifying the selection marker or detection marker.

In some embodiments, any of the vectors of the invention can comprise an enhancer sequence such as a Woodchuck Hepatitis Virus Post-transcriptional Regulatory Element (WPRE) sequence. WPRE sequences are commonly used in molecular biology to increase expression of genes delivered by viral vectors. WPRE is a tripartite regulatory element and usually is positioned at the 3′ UTR of a mammalian expression cassette to significantly increase mRNA stability and protein yield.

In some embodiments, a guide RNA vector comprises an insertion site upstream of a tracr mate sequence, and optionally downstream of a regulatory element operably linked to the tracr mate sequence, such that following insertion of a guide sequence into the insertion site and upon expression, the guide sequence directs sequence-specific binding of a CRISPR complex to a target sequence in a eukaryotic cell. In some embodiments, a guide RNA vector comprises an insertion site upstream of a tracr mate sequence, and optionally 3′ (e.g., downstream) of a regulatory element operably linked to the tracr mate sequence, such that following insertion of a guide sequence into the insertion site and upon expression, the guide sequence directs sequence-specific binding of a CRISPR complex to a target sequence in a eukaryotic cell. In some embodiments, the vector comprises two or more guide sequences. In some embodiments, the guide RNA sequences are the same. In some embodiments, the guide sequences are different. In some embodiments, the guide RNA sequences target the same gene. In some embodiments, the guide RNA sequences target different genes. When multiple different guide sequences are used, a single expression construct can be used to target CRISPR activity to multiple different, corresponding target sequences within a cell. For example, a single vector can comprise about 1, 2, 3, 4, 5, 6, 7, 8, 9, 10, 15, 20 or more guide sequences. In some embodiments, the guide RNA and the Cas are delivered using different vectors. In some embodiments, the guide RNA and the Cas are encoded on the different nucleic acids. In some embodiments, the guide RNA and the Cas are delivered using the same vector. In some embodiments, the guide RNA and the Cas are encoded on the same nucleic acid.

In some embodiments, the recognition site sequences of the guide RNA vector differ from the recognition site sequences of the Cas vector. Thus, in some embodiments, the same recombinase (e.g., Cre) can recognize and mediate recombination of the recognition site sequences of both vectors, but the recognition site sequences may be different on the two vectors (e.g., loxP and lox2272 sites) so that the recombinase does not mediate recombination between the integrated Cas and guide RNA vectors. Alternatively, different recombinases (e.g., Cre, Dre, and Flp) can recognize and mediate recombination of the recognition site sequences on the two vectors (e.g., lox and FRT sites). This strategy allows for independent excision of components of one vector (e.g., a guide RNA vector) while leaving the components of the other vector (e.g., a Cas vector) integrated. In some embodiments, this strategy could be used to integrate and excise guide RNA coding sequences sequentially while using the same integrated Cas vector to mediate RNA-guided cleavage and modification of different genetic target sites. After successful completion of all desired genetic modifications, components of the integrated Cas vector could be excised using the appropriate recombinase. In some embodiments, the nucleic acids encoding the guide RNA and the Cas are delivered using different vectors and comprise different recognition site sequences. In some embodiments, the vectors comprising the guide RNA and the Cas are encoded on different vectors and comprise different selectable markers flanked by recognition site sequences. In some embodiments, the recognition site sequences are the same. In some embodiments, the recognition site sequences are different. In some embodiments, the vectors encoding the guide RNA also encode a Cas enzyme and the selectable marker is flanked by recognition site sequences. In some embodiments, the expression of a site-specific recombinase removes the selectable marker. In some embodiments, the site-specific recombinase is Cre.

### F. Site Specific Recombinases

In some aspects, the present disclosure provides vectors comprising pairs of site-specific recombination sites flanking the coding sequences of one or more proteins that may be immunogenic to the host cell. For example, genes used for selection markers may encode proteins that are immunogenic or cause an immune response to the host organism, especially if the host organism is immunocompetent. Genes used for selectable markers may encode proteins that are immunogenic or cause an immune response to the host organism, especially if the host organism is immunocompetent. As used herein, an immunocompetent organism or host organism is an organism that is able to mount an immune response. In some embodiments, the immune response may occur after exposure to an antigen. In some embodiments, the antigen is an antigen associated with a pathogen. In some embodiments, the antigen is an antigen associated with a vaccine. In some embodiments, an immunocompetent organism or host organism is an organism that has an immune system that can be activated by an antigen. In some embodiments, the antigen is Cas. In some embodiments, the antigen is GFP. In some embodiments, the immunocompetent organism or host organism is tolerized to an antigen. In some embodiments, the antigen is Cas. In some embodiments, the antigen is GFP. In some embodiments, the immunocompetent organism is tolerized to Cas and/or GFP, such that it does not mount an immune response to Cas and/or GFP. Selectable markers are almost invariably derived from non-mammalian species, exogenous introduction of these proteins into immunocompetent mice poses the risk of immunological rejection by host-derived T cells. Multiple cases of immunological rejection of cells expressing RPs, such as firefly luciferase and EGFP, have been reported. Moreover, major histocompatibility complex (MHC) class I-restricted epitopes of luciferase and GFP have been identified, underlining their capacity to induce CD8+ T-cell responses. Even in the absence of complete immunological rejection of cells comprising selectable markers, experimental outcomes may potentially be subtly biased through the action of such undesirable immune responses.

Site-specific recombination sites, as used herein, refer to DNA sequences that are typically between 30 and 200 nucleotides in length and consist of two motifs with a partial inverted-repeat symmetry, to which a site-specific recombinase binds and mediates recombination. Site-specific recombinases, as used herein, refers to a group of enzymes that catalyze directionally sensitive DNA exchange reactions between target site sequences that are specific to each recombinase. Non-limiting examples of site specific recombinase-site specific recombination sites pairs include Cre-Lox, Flp-FRT, ΦC31-attP/attB, and Dre-Rox. Thus, in some embodiments, the recombinase is Cre, Flp, ΦC31 or Dre, and in some embodiments, the site-specific recombination sites are lox, FRT, attP/attB and rox, respectively.

In some embodiments, the site-specific recombination sites are lox sites. Lox sites are typically about 34 base pairs and consist of two palindromic regions of about 13 bp and an intervening non-palindromic spacer of about 8 bp that determines the orientation of the site. When two lox sites are oriented in the same direction, the site-specific recombinase Cre excises the DNA flanked by the lox sites, leaving a single lox site behind.

The site-specific recombinases of the invention can be introduced to the host cells by any means known in the art, including the various delivery vectors described herein. However, because they can be expressed more transiently, in some embodiments non-integrating vectors (e.g., IDLV vectors, smaller expression vectors such as SV40 or AAV vectors) or physical or chemical techniques of introducing nucleic acids (e.g., electroporation, biolistic particles) can be preferred. In addition, although detectable markers can be included in recombinase vectors, such markers may not be necessary if recombinase-mediated excision of Cas vector or guide RNA vector components includes excision of a detectable marker in one of those vectors.

Differences in palindromic or spacer regions of lox sites, either naturally-occurring or randomly mutated, can confer specificity to Cre recognition. Non-limiting examples of mutated lox sites are loxP511, lox2272, loxΔ86, loxΔ117, loxC2, loxP2, loxP3, loxP23, loxB, loxL and loxR, all of which are known in the art. In some embodiments, the lox sites are loxP sites. In some embodiments, the lox sites are mutated lox sites. In some embodiments, the mutated lox sites are lox2272. In other embodiments, the mutated lox sites are lox5171. The Lox-Cre system is disclosed in further detail in Sauer, B. (1987), Mol Cell Biol. 7 (6): 2087-2096; Tsien, Joe Z. (2016). Frontiers in Genetics. 7: 19; Shakes et al., Nucleic Acids Res. 2005; 33(13): e118; R H Hoess, M Ziese, & N Sternberg, PNAS Jun. 1, 1982, 79(11): 3398-3402; Michel G, et al., Mol Ther. 2010; 18(10):1814-21; and U.S. Pat. Nos. 6,828,093 and 7,179,644, each of which is incorporated herein by reference.

In some embodiments, the site-specific recombination sites are FRT sites. The FRT sites are about 34 bp and consist of two palindromic regions of about 13 bp and an intervening non-palindromic core region of about 8 bp that determines the orientation of the site. Several variant FRT sites exist, but recombination can usually occur only between two identical FRTs and not among non-identical or “heterospecific” FRTs. When two FRT sites are oriented in the same direction, the site-specific recombinase Flp can excise the DNA flanked by the FRT sites, leaving a single FRT site behind. See Schubeler D, Maass K & Bode J, Biochemistry. 1998 Aug. 25; 37(34):11907-14, incorporated herein by reference.

In some embodiments, the site-specific recombination sites are attL and attR sites. The attL and attR sites are recognized by the ΦC31 integrase, a site-specific bacteriophage recombinase. See Pokhiliko et al., Nucleic Acids Res. 2016; 44(15): 7360-7372, incorporated herein by reference.

In some embodiments, the site-specific recombination sites are rox sites. The rox sites are recognized by Dre recombinase. Dre recombinase is a bacteriophage-derived tyrosine recombinase that recognizes a pair of identical rox sites and leaves behind a single rox site after recombination. See Anastassiadis K et al., Disease Models & Mechanisms 2009 2: 508-515, incorporated herein by reference.

In some embodiments at least one selectable marker is flanked by the site-specific recombination sites. In some embodiments, at least one detectable marker is flanked by the site-specific recombination sites. In some embodiments, the site-specific recombination sites also flank at least some other components, such as promoters, spacers, enhancers, multiple cloning sites, IRES, WPRE, etc.

In some embodiments the detection marker is flanked the site-specific recombination sites. In some embodiments the detection marker is flanked by the site-specific recombination sites. In some embodiments, at least one detectable marker is flanked by the site-specific recombination sites. In some embodiments, the site-specific recombination sites also flank at least some other components, such as promoters, spacers, enhancers, multiple cloning sites, IRES, WPRE, etc.

In order to excise the nucleotide sequences flanked by the recognition sites, a site-specific recombinase that catalyzes the recombination between the site-specific recombination sites needs to be delivered the cells. In some embodiments, the recombinase is delivered through mRNA transfection. In some embodiments, the recombinase is delivered as a protein. In some embodiments, the recombinase is delivered by a delivery vector. In some embodiments, the recombinase is delivered by an expression vector. In some embodiments, the recombinase is delivered by AAV vector. In other embodiments, the recombinase is delivered by an integrase deficient lentiviral vector. In some embodiments, the recombinase is Cre. In some embodiments, the recombinase is delivered with a detection marker. In some embodiments, the detection marker is mCherry or RFP. In some embodiments, the recombinase is transiently expressed. In some embodiments, the recombinase is not present in the cell after the selection marker and/or detection marker is excised. In some embodiments, the recombinase is not present in the cell after the selectable marker and/or detection marker is excised.

### G. Types of Vectors

In some embodiments, a recombinant expression vector of the present disclosure is a viral vector. In some embodiments, the recombinant expression vector is a recombinant retroviral vector. In some embodiments, the retroviral vector is a lentivirus vector. In some embodiments, the recombinant expression vector is a non-retroviral vector. In some embodiments, the recombinant expression vector of the present disclosure is an adenovirus vector.

Retroviral vectors can be derived from any of the Alpharetroviruses, Betaretroviruses, Gammaretroviruses, Deltaretroviruses, Epsilonretroviruses, or Lentiviruses. At present, the Gammaretroviruses and the Lentiviruses have been most studied and adapted for use in genetic engineering and gene therapy, being especially important the vectors derived from human immunodeficiency virus (HIV)-1. For safety, the viruses are modified to make them replication defective and, therefore, they may be produced with the aid of packaging plasmids or packaging cell lines. Thus, common modifications included in retroviral vectors are deletion and/or inactivation of one or more of the gag, pol and end proteins which are necessary for replication.

Lentiviruses can be classified into five families (1) primate, (2) bovine, (3) ovine/caprine, (4) equine and (5) feline. Lentiviral vectors derived from primate lentiviruses are preferred in the present disclosure, although other lentiviral vectors may be used.

For brevity, the following discussion focuses on lentiviral vectors, although it will be apparent to those of skill in the art that it applies to retroviral vectors generally and that other retroviral vectors fall within the scope of the invention.

Lentiviruses have been developed as efficient delivery vectors for gene therapy and genome editing because they can integrate a significant amount of viral cDNA into the genome of a host cell and because they can infect non-dividing cells. Lentivirus particles contain two single-stranded positive sense RNA-genomes. The native lentivirus genome is approximately 10 kb long and is flanked by long terminal repeats (LTRs). A sequence located near the 5′ end of the genome, known as the Psi (Ψ) packaging element, is necessary for packaging viral RNA into capsids and, therefore, is included in the vectors of the invention. For simplicity, the Psi element is omitted from some figures but is understood to be present immediately 3′ of the 5′ LTR. Transgenes intended for integration by lentiviral vectors may be included between the 5′ Psi sequence and the 3′ LTR.

Prior to integration into a host genome, the lentiviral RNA genome may be converted into DNA by a reverse transcriptase that synthesizes a first strand of DNA from the RNA genome. A host cell DNA polymerase then synthesizes the second strand to produce a double-stranded DNA. Integration of the vector is mediated by an integrase and the LTRs. Lentiviral LTRs typically comprise about 600 nucleotides and include distinct U3, R and U5 regions.

Prior to integration, certain LTR elements are duplicated during reverse transcription. Specifically, the U3 region in the 3′ LTR region is copied and incorporated into the 5′ LTR. Thus, if part of the U3 region in the 3′ LTR is deleted, the same deletion will be duplicated into the 5′ LTR. Similarly, if a nucleotide sequence is inserted into the U3 region of the 3′ LTR (e.g., a site-specific recombination site or a hU6 guide expression cassette), the same insertion will be duplicated into the 5′ LTR during reverse transcription of the viral RNA genome. Thus, after integration, such deletions/insertions will be present in both the 5′ and 3′ LTRs of the provirus.

Lentiviral vectors are produced by modifying lentiviruses such that they are replication defective but still capable of integration, have deletions of one or more loci which are not necessary for their role as a vector (e.g., deletion or inactivation of the gag, pol and env loci needed for replication), and insertion of one or more transgenes which are necessary or useful for their role as a vector for genome-editing (e.g., a Cas enzyme coding sequence, detectable markers).

The CRISPR/Cas lentiviral vectors of the invention are reproduction or replication defective, but are not integration deficient. Thus, the vectors can integrate into a host genome but cannot reproduce themselves. Therefore, the vectors may be produced by transfecting the lentiviral vector with one or more plasmids that encode the viral components necessary to produce an infectious viral particle, including proteins necessary for produced viral capsids and packaging viral genomes into the capsids. A variety of such packaging systems, including packaging plasmids or packaging cell lines, are known in the art and widely available. The most commonly used systems are known as second and third generation lentiviral packaging systems.

In some embodiments, the lentiviral vector can be paired with a second generation packaging system. Such second generation lentiviral packaging systems can include a single packaging plasmid encoding the Gag, Pol, Rev, and Tat genes. The lentiviral vector of the invention will include the viral LTRs, Psi packaging signal and transgenes (e.g., Cas, detectable marker(s)). Unless an internal promoter is provided (e.g., “Promoter 1” as described above), gene expression is driven by the 5′ LTR, which is a weak promoter and may require the presence of Tat to activate expression. The envelope protein Env (usually VSV-G due to its wide infectivity) can be encoded on a third, separate, envelope plasmid. Non-limiting examples of second generation lentiviral packaging plasmids include psPAX2, pCMV delta R8.2, pCMV-dR8.2 dvpr, pCPRDEnv, pCD/NL-BH*DDD, psPAX2-D64V, and pNHP. Non-limiting examples of second generation lentiviral envelope plasmids include pMD2.G, pCMV-VSV-G, pLTR-RD114A, and pLTR-G.

In some embodiments, the lentiviral vector can be paired with a third generation packaging system. The third generation systems further improve on the safety of the second generation systems in several ways. First, the packaging plasmid is split into two plasmids: one encoding Rev and one encoding Gag and Pol. Second, Tat is eliminated from the third generation system through the addition of a chimeric 5′ LTR fused to a heterologous promoter on the transfer plasmid. Expression of the transgene(s) from this promoter is not dependent on Tat transactivation. The third generation vectors can be packaged by either a second generation or third generation packaging system. Non-limiting examples of the third generation lentiviral packaging plasmids include pRSV-Rev, and pMDLg/pRRE.

In some embodiments, the guide RNA and/or site-specific recombinase transgenes are delivered by non-retroviral vectors, such as SV40 or adeno-associated virus (AAV) vectors.

One major advantage of using AAV for research is that it is replication-limited and typically not known to cause disease in humans. For these reasons, AAVs are generally contained at lower biosafety levels and elicit relatively low immunological effects in vivo. AAV can transduce both dividing and non-dividing cells with a low immune response and low toxicity. Although recombinant AAV does not integrate into the host genome, transgene expression can be long-lived. The utility of AAV is currently limited by its small packaging capacity (~4.5 kb including inverted terminal repeats (ITRs)), though there is a great deal of interest and effort directed toward expanding this capacity. The small (4.8 kb) ssDNA AAV genome consists of two open reading frames, Rep and Cap, flanked by two 145 base ITRs. These ITRs base pair to allow for synthesis of the complementary DNA strand. Rep and Cap are translated to produce multiple distinct proteins (Rep78, Rep68, Rep52, Rep40—required for the AAV life cycle; VP1, VP2, VP3—capsid proteins). When constructing an AAV transfer vector, the transgene is placed between the two ITRs, and Rep and Cap are supplied in trans. In addition to Rep and Cap, AAV requires a helper plasmid containing genes from adenovirus. These genes (E4, E2a and VA) mediate AAV replication. The transfer plasmid, Rep/Cap, and the helper plasmid are commonly transfected into cells such as HEK293 cells, which contain the adenovirus gene E1+, to produce infectious AAV particles. Rep/Cap and the adenovirus helper genes can also be combined into a single plasmid. Eleven serotypes of AAV have thus far been identified, with the best characterized and most commonly used being AAV2. These serotypes differ in their tropism, or the types of cells they infect, making AAV a very useful system for preferentially transducing specific cell types.

In some embodiments, the vector comprises a nucleic acid that encodes a Cas enzyme. In some embodiments, the nucleic acid comprises a multiple cloning site upstream of a Cas enzyme. In some embodiments, the nucleic acid comprises, from 5′ to 3′, a 5′ LTR, a multiple cloning site, a Cas enzyme, a recognition site, an IRES site, a selection marker, a WPRE element, a recognition site, and a 3′ LTR. In some embodiments, a promoter is cloned into the multiple cloning site. In some embodiments, the nucleic acid comprises a promoter that is upstream and operably linked to a sequence encoding a Cas. In some embodiments, the nucleic acid comprises, from 5′ to 3′, 5′ LTR, a multiple cloning site, a sequence encoding a Cas enzyme, a recognition site, an IRES site, a selection marker, a WPRE element, another recognition site, and a 3′ LTR. In some embodiments, the promoter is a human CMV promoter, a human PGK promoter, a mouse PGK promoter, a human EF1a promoter, or a mouse EF1a promoter. In some embodiments, the Cas enzyme is a Cas9. In some embodiments, the recognition site is a loxP site. In some embodiments, the selection marker is the BSD gene (e.g., encoding resistance to blasticidin). In some embodiments, the selectable marker is removed upon expression of Cre.

In some embodiments, the vector comprises a nucleic acid that encodes a Cas enzyme. In some embodiments, the nucleic acid comprises a multiple cloning site upstream of a Cas enzyme. In some embodiments, the nucleic acid comprises, from 5′ to 3′, a 5′ LTR, a multiple cloning site, a Cas enzyme, a recognition site, an IRES site, a selectable marker, a WPRE element, a recognition site, and a 3′ LTR. In some embodiments, a promoter is cloned into the multiple cloning site. In some embodiments, the nucleic acid comprises a promoter that is upstream and operably linked to a sequence encoding a Cas. In some embodiments, the nucleic acid comprises, from 5′ to 3′, 5′ LTR, a multiple cloning site, a sequence encoding a Cas enzyme, a recognition site, an IRES site, a selectable marker, a WPRE element, another recognition site, and a 3′ LTR. In some embodiments, the promoter is a human CMV promoter, a human PGK promoter, a mouse PGK promoter, a human EF1a promoter, or a mouse EF1a promoter. In some embodiments, the Cas enzyme is a Cas9. In some embodiments, the recognition site is a loxP site. In some embodiments, the selectable marker is the BSD gene (e.g., encoding resistance to blasticidin). In some embodiments, the selectable marker is removed upon expression of Cre.

In some embodiments, the vector comprises a nucleic acid that encodes a guide RNA. In some embodiments, the nucleic acid comprises, from 5′ to 3′, a PolIII promoter, a nucleic acid encoding a guide RNA, a detection marker, a recognition site, an IRES site, a selection marker, a WPRE element, and another recognition site. In some embodiments, the PolIII promoter that is upstream and operably linked to a sequence encoding the guide RNA. Therefore in some embodiments, the nucleic acid comprises, from 5′ to 3′, a 5′ LTR, a PolIII promoter, a first guide RNA, a detection marker, a recognition site, an IRES site, a selection marker, a WPRE element, a recognition site, a PolIII promoter, a second guide RNA, and a 3′ LTR. In some embodiments, the first guide RNA is the same as the second guide RNA. In some embodiments, the first guide RNA is different from the second guide RNA. In some embodiments, the first guide RNA targets the same gene as the second guide RNA. In some embodiments, the first guide RNA targets a different gene from the second guide RNA. In some embodiments, the detection marker is a fluorescent protein. In some embodiments, the detection marker is an EGFP. In some embodiments, the recognition site is a lox2772 site. In some embodiments, the selection marker is the PAC gene (e.g., encoding resistance to puromycin). In some embodiments, the selectable marker is removed upon expression of Cre.

In some embodiments, the vector comprises a nucleic acid that encodes a guide RNA. In some embodiments, the nucleic acid comprises, from 5′ to 3′, a PolIII promoter, a nucleic acid encoding a guide RNA, a detection marker, a recognition site, an IRES site, a selectable marker, a WPRE element, and another recognition site. In some embodiments, the PolIII promoter that is upstream and operably linked to a sequence encoding the guide RNA. Therefore in some embodiments, the nucleic acid comprises, from 5′ to 3′, a 5′ LTR, a PolIII promoter, a first guide RNA, a detection marker, a recognition site, an IRES site, a selectable marker, a WPRE element, a recognition site, a PolIII promoter, a second guide RNA, and a 3′ LTR. In some embodiments, the first guide RNA is the same as the second guide RNA. In some embodiments, the first guide RNA is different from the second guide RNA. In some embodiments, the first guide RNA targets the same gene as the second guide RNA. In some embodiments, the first guide RNA targets a different gene from the second guide RNA. In some embodiments, the detection marker is a fluorescent protein. In some embodiments, the detection marker is an EGFP. In some embodiments, the recognition site is a lox2772 site. In some embodiments, the selectable marker is the PAC gene (e.g., encoding resistance to puromycin). In some embodiments, the selectable marker is removed upon expression of Cre.

### Systems

Several aspects of the invention relate to vector systems, which comprising one or more vectors, or vectors as such. The invention relates to the vectors systems that comprise viral vectors comprising one or more components of a gene editing system (e.g., a Cas enzyme and/or a guide RNA). In some embodiments, the system comprises nucleic acid encoding a Cas enzyme, a guide RNA, and one or more selectable markers flanked by recognition sites for a site-specific recombinase. In some embodiments, the system comprises two or more vectors.

In some embodiments, the system comprises a vector comprising a nucleic acid comprising a Cas enzyme and a selectable marker. In some embodiments, the system comprises a vector comprising a nucleic acid comprising a guide RNA and a selectable marker. In some embodiments, the system comprises a vector comprising a nucleic acid comprising a guide RNA, a selectable marker and a detection marker. In some embodiments, the system comprises a vector comprising a nucleic acid comprising a Cas enzyme, one or more guide RNAs, a selectable marker and a detection marker. In some embodiments, the system comprises two or more vectors, wherein the vectors comprise nucleic acids that comprise one or more selectable markers, at least one detection marker, a Cas enzyme, and one or more guide RNAs.

In some embodiments, the system comprises a vector comprising a nucleic acid encoding the Cas enzyme, the nucleic acid encoding the guide RNA, and the nucleic acid encoding the one or more selectable markers comprising transducing or transfecting the cell with one or more vectors comprising the nucleic acid encoding the guide RNA, and the nucleic acid encoding the selectable marker. In some embodiments, the system comprises two or more vectors. In some embodiments, the two or more vectors comprises a selectable marker. In some embodiments, the two or more vectors comprising two or more selectable markers. In some embodiments, the vectors comprise two selectable markers. In some embodiments, the vectors comprise a first selectable marker and a second selectable marker. In some embodiments, the first selectable and the second selectable markers are different. In some embodiments, the selectable markers are flanked by recognition sites for the site-specific recombinase.

In some embodiments, the system comprising the nucleic acid encoding the Cas enzyme and the nucleic acid encoding the guide RNA are provided on two different vectors. In some embodiments the one vector (e.g., a first vector) comprises the nucleic acid encoding the Cas enzyme and nucleic acid encoding a first selectable marker flanked by recognition sites for a site specific-recombinase, and wherein another vector (e.g., a second vector) comprises the nucleic acid encoding the guide RNA and nucleic acid encoding a second selectable marker flanked by recognition sites for a site-specific recombinase. In some embodiments, the system comprises a first vector comprising the nucleic acid encoding the Cas enzyme and a first selectable marker and a second vector comprising the nucleic acid encoding the guide RNA and a second selectable marker. In some embodiments, the selectable markers (e.g., the first and/or the second selectable marker) are removed following selecting the cells for the selectable marker (e.g., the first and/or the second selectable marker). In some embodiments, the selectable markers (e.g., the first and/or the second selectable marker) are removed using a site-specific recombinase. In some embodiments, the cells are further selected for the selectable marker (e.g., the first and/or the second selectable marker). In some embodiments, the cells are selected for the selectable marker (e.g., the first and/or the second selectable marker) comprising culturing the cell with an antibiotic. In some embodiments, the selectable markers (e.g., the first and/or the second selectable marker) are removed using a site-specific recombinase after selection for cells comprising the selectable marker (e.g., the first and/or the second selectable marker). In some embodiments, the cells are further cultured following selection with the selectable marker (e.g., the first and/or the second selectable marker). In some embodiments, the nucleic acid encoding the guide RNA is not removed. In some embodiments, the nucleic acid encoding the guide RNA is operably linked to a PolII promoter. In some embodiments, one or more vectors comprises nucleic acid encoding a detection marker. In some embodiments, the nucleic acid encoding the detection marker is flanked by recognition sites for the site specific recombinase. In some embodiments, the detection marker is a fluorescent protein. In some embodiments, the fluorescent protein is a GFP, YFP, RFP, EGFP, or a luciferase. In some embodiments, the fluorescent protein is EGFP. In some embodiments, the detection marker is detected in the cell. In some embodiments, the nucleic acid encoding the Cas enzyme is operably linked to a promoter. In some embodiments, the promoter is an inducible promoter. In some embodiments, the promoter is a human or a murine promoter. In some embodiments, the Cas enzyme is Cas9.

In some embodiments, the system comprising the nucleic acid encoding the Cas enzyme and the nucleic acid encoding one or more guide RNAs are provided on one, two, three, four, or more different vectors. for example, the number of guide RNAs delivered to each cell can, on average, be greater than 1, 2, 3, 4, 5, 6, 7, 8, 9, 10 or higher. In some embodiments the one vector (e.g., a first vector) comprises the nucleic acid encoding the Cas enzyme and nucleic acid encoding a first selectable marker flanked by recognition sites for a site specific-recombinase, and wherein another vector (e.g., a second, third, or fourth vector) comprises the nucleic acid encoding the guide RNAs and nucleic acid encoding selectable markers flanked by recognition sites for a site-specific recombinase. In some embodiments, the selectable markers are removed following selecting the cells for the selectable marker. In some embodiments, the selectable markers are removed using a site-specific recombinase. In some embodiments, the cells are further selected for the selectable marker. In some embodiments, the cells are selected for the selectable marker comprising culturing the cell with an antibiotic. In some embodiments, the selectable markers are removed using a site-specific recombinase after selection for cells comprising the selectable marker. In some embodiments, the cells are further cultured following selection with the selectable marker. In some embodiments, the nucleic acid encoding the guide RNA is not removed. In some embodiments, the nucleic acid encoding the guide RNA is operably linked to a PolIII promoter. In some embodiments, one or more vectors comprises nucleic acid encoding a detection marker. In some embodiments, the nucleic acid encoding the detection marker is flanked by recognition sites for the site specific recombinase. In some embodiments, the detection marker is a fluorescent protein. In some embodiments, the fluorescent protein is a GFP, YFP, RFP, EGFP, or a luciferase. In some embodiments, the fluorescent protein is GFP. In some embodiments, the detection marker is detected in the cell. In some embodiments, the nucleic acid encoding the Cas enzyme is operably linked to a promoter. In some embodiments, the promoter is an inducible promoter. In some embodiments, the promoter is a human or a murine promoter. In some embodiments, the Cas enzyme is Cas9.

### Engineered Cells

The present disclosure relates to an engineered cell comprising one or more components of a gene editing system such as a nucleic acid encoding a Cas and a nucleic acid encoding a guide RNA along with one or more selectable markers flanked by recombination sites for a site-specific recombinase. In some embodiments, the engineered cell comprises a vector comprising a nucleic acid encoding a Cas enzyme and a selectable marker. In some embodiments, the engineered cell comprises a vector comprising a nucleic acid comprising a guide RNA and a selectable marker. In some embodiments, the engineered cell comprises a vector comprising a nucleic acid comprising a guide RNA, a selectable marker and a detection marker. In some embodiments, the engineered cell comprises Cas and a selectable marker, and the selectable marker is removed but the Cas remains. In some embodiments, the engineered cell comprises a vector comprising a guide RNA and a selectable marker, and the selectable marker is removed but the guide RNA remains. In some embodiments, the engineered cell comprises a vector comprising a guide RNA, a selectable marker and a detection marker, and the selectable marker is removed but the guide RNA remains. In some embodiments, the engineered cell comprises a vector comprising a guide RNA, a selectable marker and a detection marker, and the detection marker is removed but the guide RNA remains. In some embodiments, the engineered cell comprises a vector comprising a guide RNA, a selectable marker and a detection marker, and the selectable marker and the detection marker is removed but the guide RNA remains. In some embodiments, the disclosure relates to a plurality of cells comprising the cell.

In some embodiments, the present disclosure relates to an engineered cell comprising a vector as disclosed in any of the sections above. The cells comprise the viral vectors comprising one or more components of a gene editing system (e.g., a Cas enzyme and/or a guide RNA). The cells may also be contacted with the viral vectors comprising one or more components of a gene editing system (e.g., a Cas enzyme and/or a guide RNA), then further modified with a site-specific recombinase. Cells that have utility in the present invention may be murine or human in origin. Human or murine cell line known to one of ordinary skill in the art may also be used as a host cell.

In some embodiments, the engineered cell is provided the nucleic acid encoding the Cas enzyme, the nucleic acid encoding the guide RNA, and the nucleic acid encoding the one or more selectable markers comprising transducing or transfecting the cell with one or more vectors comprising the nucleic acid encoding the guide RNA, and the nucleic acid encoding the selectable marker. In some embodiments, the cell is provided two or more vectors. In some embodiments, the two or more vectors comprise each comprise a selectable marker. In some embodiments, the selectable marker are different. In some embodiments, the selectable markers are flanked by recognition sites for the site-specific recombinase. In some embodiments, the nucleic acid encoding the Cas enzyme and the nucleic acid encoding the guide RNA are provided on two different vectors. In some embodiments the one vector (e.g., a first vector) comprises the nucleic acid encoding the Cas enzyme and nucleic acid encoding a first selectable marker flanked by recognition sites for a site specific-recombinase, and wherein another vector (e.g., a second vector) comprises the nucleic acid encoding the guide RNA and nucleic acid encoding a second selectable marker flanked by recognition sites for a site-specific recombinase. In some embodiments, the selectable markers are removed following selecting the cells for the selectable marker. In some embodiments, the selectable markers are removed using a site-specific recombinase. In some embodiments, the engineered cells are further selected for the selectable marker. In some embodiments, the engineered cells are selected for the selectable marker comprising culturing the engineered cell with an antibiotic. In some embodiments, the selectable markers are removed using a site-specific recombinase after selection for engineered cells comprising the selectable marker. In some embodiments, the cells are further cultured following selection with the selectable marker. In some embodiments, the nucleic acid encoding the guide RNA is not removed. In some embodiments, the nucleic acid encoding the guide RNA is operably linked to a PolIII promoter. In some embodiments, one or more vectors comprises nucleic acid encoding a detection marker. In some embodiments, the nucleic acid encoding the detection marker is flanked by recognition sites for the site specific recombinase. In some embodiments, the detection marker is a fluorescent protein. In some embodiments, the fluorescent protein is a GFP, YFP, RFP, EGFP, or a luciferase. In some embodiments, the fluorescent protein is GFP. In some embodiments, the detection marker is detected in the cell. In some embodiments, the nucleic acid encoding the Cas enzyme is operably linked to a promoter. In some embodiments, the promoter is an inducible promoter. In some embodiments, the promoter is a human or a murine promoter. In some embodiments, the Cas enzyme is Cas9. In some embodiments, the cell is a human or a murine cell. In some embodiments, the cell is a cell from a primary tumor or a tumor cell line. In some embodiments, the cell is a murine cell derived from a syngeneic (genetically similar or identical and hence immunologically compatible) mouse. In some embodiments, the cell is a MC38 cell. In some embodiments, the cell is a B16F10 cell.

In some embodiments, the engineered cell comprises a nucleic acid encoding a Cas enzyme after removing from the selected cells the nucleic acid encoding the selectable marker from the vector, wherein the nucleic acid encoding the Cas enzyme is maintained. In some embodiments, the engineered cell comprises a nucleic acid encoding a Cas enzyme after removing nucleic acid encoding a selectable marker from a vector in the engineered cell, wherein the vector comprises a nucleic acid encoding a Cas enzyme, a nucleic acid encoding a guide RNA, and the nucleic acid encoding the selectable marker, wherein the nucleic acid encoding the Cas enzyme is maintained. In some embodiments, the nucleic acid encoding the selectable marker is removed by providing a site-specific recombinase to the engineered cell. In some embodiments, the site-specific recombinase is selected from the group consisting of Cre, Dre, and Flp. In some embodiments, providing the site-specific recombinase to the engineered cell comprises providing protein or mRNA encoding the site-specific recombinase to the engineered cell. In some embodiments, the Cas enzyme is Cas9. In some embodiments, the engineered cell is a human or a murine cell. In some embodiments, the engineered cell is a cell from a primary tumor or a tumor cell line. In some embodiments, the engineered cell is a murine cell derived from a syngeneic (e.g., genetically similar or identical and hence immunologically compatible) mouse. In some embodiments, the engineered cell is a MC38 cell. In some embodiments, the engineered cell is a B16F10 cell.

### Methods of Generating Engineered Cells

In some embodiments, the present invention relates to methods that generate engineered cells comprising CRISPR/Cas gene editing components that have lower immunogenicity. In some embodiments, the methods comprise removal of one or more selectable markers present on a vector that comprises a CRISPR/Cas gene editing component, following selection for the marker. The present disclosure provides methods for producing genetically modified (e.g., engineered) cells using a CRISPR/Cas system with one or more vectors that undergo site-specific recombination to excise at least some immunogenic components of the vectors of the cells. In some embodiments, the cells are genetically modified using CRISPR/Cas gene editing. In some embodiments, the gene editing occurs after the cell has been introduced into an organism. In some embodiments, the organism is a mouse.

In some embodiments, provided herein are methods generating engineered cells comprising a nucleic acid encoding a Cas enzyme comprising (i) selecting a plurality of cells comprising a nucleic acid encoding a Cas enzyme, a nucleic acid encoding a guide RNA, and a nucleic acid encoding a selectable marker, and (ii) removing from the selected cells the nucleic acid encoding the selectable, wherein the resulting engineered cells comprise the nucleic acid encoding the Cas enzyme and the nucleic acid encoding the guide RNA.

In some embodiments, the method of generating engineered cells comprise providing a population of cells, introducing any of the vectors encoding a Cas enzyme as described above into the cells, introducing any of the vectors encoding at least one guide RNA into the cells, culturing the population of cells for a time sufficient for selection, removing the cells from selection, introducing a mRNA encoding a site-specific recombinase using transfection, culturing the population of cells for a time sufficient for the recombinase to be expressed and excised, wherein the recombinase catalyzes recombination between the first 3′ site-specific recombination site and a first 5′ site-specific recombination site located 3′ to at least the selectable marker, thereby causing excision of the selection marker sequence from the genomes of at least a portion of the population of cells. In some embodiments, the cell has been edited by a Cas enzyme.

In some embodiments, the method of generating engineered cells comprise providing a population of cells, introducing any of the vectors encoding a Cas enzyme as described above into the cells, introducing any of the vectors encoding at least one guide RNA into the cells, culturing the population of cells for a time sufficient for selection, removing the cells from selection, introducing a mRNA encoding a site-specific recombinase using transfection, culturing the population of cells for a time sufficient for the recombinase to be expressed and excised, wherein the recombinase catalyzes recombination between the first 3′ site-specific recombination site and a first 5′ site-specific recombination site located 3′ to at least the selectable marker, thereby causing excision of the selectable marker sequence from the genomes of at least a portion of the population of cells. In some embodiments, the cell has been edited by a Cas enzyme.

In some embodiments, the method of generating engineered cells comprises providing a population of cells, introducing any of the vectors encoding a Cas enzyme as described above into the cells, introducing any of the vectors encoding at least one guide RNA into the cells, culturing the population of cells for a time sufficient for detection, identifying cells containing the vectors, introducing a mRNA encoding a site-specific recombinase using transfection, culturing the population of cells for a time sufficient for the recombinase to be expressed and excised, wherein the recombinase catalyzes recombination between the first 3′ site-specific recombination site and a first 5′ site-specific recombination site located 3′ to at least the detection marker, thereby causing excision of the selectable marker sequence from the genomes of at least a portion of the population of cells.

In some embodiments, the invention provides the method of generating engineered cells, wherein the cells have been genetically-modified at a target. Specifically, in some embodiments, one guide RNAs complementary to a genetic target can be introduced into the population of cells such that, the target site is modified in each cell. For example, the number of guide RNA vectors delivered to each cell can be 1. This will result in generating a cell genetically-modified at one genetic target.

In some embodiments, the invention provides the method of generating engineered cells, wherein a population of cells have been genetically-modified at more than one target. Specifically, in some embodiments, one guide RNAs complementary to a genetic target can be introduced into the population of cells such that, the target sites are modified in each cell. For example, the number of guide RNA vectors delivered to each cell can, on average, be greater than 1, 2, 3, 4, 5, 6, 7, 8, 9, 10 or higher. In addition, the number of different types or species of guide RNAs delivered to the population of cells can be greater than 1, 10, 10², 10³, 10⁴ or higher. This will result in generating a population of cells genetically-modified at more than one genetic target.

Such cell with one or more genetic modifications can be useful in identifying the role of the target in complex diseases including cancer. For example, the cell with the genetic knockout can be used for target validation in immune-competent mice, which is important for complex oncology targets. It can also be used to develop inducible in vivo gene-editing systems. Complex targets require intact tumor context (e.g., tumor cell interactions, stromal context, immune infiltration/modulation) and requires implantation of CRISPR engineered cells in immune-competent mice.

In some embodiments, the invention provides methods for generating a cell that has been genetically modified or epigenetically at a target DNA site that is complimentary to at least part of a guide RNA. In some embodiments, the modification is effectuated by a Cas enzyme. In some embodiments, the genetic modification comprises cleavage or cutting of the DNA or RNA strand. In some embodiments, the genetic modification is insertion, deletion, duplication of DNA. In some embodiments, the genetic modification is editing at a single nucleotide base. In some embodiments, the genetic modification is editing at one or more nucleotide bases. In some embodiments, the genetic modification is homologous recombination of the DNA. In some embodiments, the genetic modification is nicking of the target DNA. In some embodiments, the epigenetic modification is a chemical modification. Chemical modifications include but are not limited to cytosine deamination or DNA methylation. In some embodiments, the epigenetic modification is an addition of a protein moiety. In some embodiments, the epigenetic modification is the addition of ubiquitin. In some embodiments, the epigenetic modification is effectuated or mediated by the binding of a dead Cas or a fusion or variant thereof.

In some embodiments, the invention provides for methods of generating engineered cells has been genetically modified at a variety of genetic targets. Specifically, in some embodiments, a variety of different types or species of guide RNAs complementary to a variety of different genetic targets can be introduced into the population of cells such that, on average, more than one target site is modified in each cell. For example, the number of guide RNA vectors delivered to each cell can, on average, be greater than 1, 2, 3, 4, 5, 6, 7, 8, 9, 10 or higher. In addition, the number of different types or species of guide RNAs delivered to the population of cells can be greater than 1, 10, 10², 10³, 10⁴ or higher. This will result in a population or pool of genetically modified cells in which most cells will be genetically-modified at more than one genetic target and in which there are many types or subsets of cells with different combinations of modified targets. For example, with 10 targets (or, more generally, X targets) and each cell being modified at exactly two different target sites, there would be 45 possible combinations of modified targets (or, more generally, X(X−1)/2), and for 10³ targets there would be 499,500. With more guide RNA vectors delivered to each cell (i.e., similar to a higher multiplicity of infection) and more types or species of guide RNA vectors, an incredibly diverse or complex pool of genetically-modified cells can be produced.

Such cell with multiple genetic modifications can be useful in identifying the role of the target in complex diseases including cancer. For example, CRISPR engineered cells express multiple exogenous immunogenic proteins may be used to study disease progression in an intact tumor context (e.g., tumor cell interactions, stromal context, immune infiltration/modulation) and requires implantation of CRISPR engineered cells in immune-competent mice.

In some embodiments, the present disclosure provides a method of generating a cell comprising a nucleic acid encoding a Cas enzyme comprising selecting cells comprising one or more vectors comprising a nucleic acid encoding a Cas enzyme, a nucleic acid encoding a guide RNA, and a nucleic acid encoding a selectable marker for the selectable marker, and removing from the selected cells, the nucleic acid encoding the selectable marker from the vector, wherein the nucleic acid encoding the Cas enzyme is maintained. In some embodiments, the nucleic acid encoding the selectable marker is removed by providing a site-specific recombinase to the cell. In some embodiments, the site-specific recombinase is provided to the cell by introducing protein or mRNA encoding the site-specific recombinase. In some embodiments, the mRNA is produced by performing in vitro transcription to produce the mRNA. In some embodiments, the method further comprises detecting removal of the selectable marker. Non-limiting examples of methods to detect removal of the selectable marker include qPCR.

In some embodiments, the present disclosure provides for methods of generating engineered cells comprising a nucleic acid encoding a Cas enzyme comprising removing nucleic acid encoding a selectable marker from a vector in a cell, wherein the vector comprises a nucleic acid encoding a Cas enzyme, a nucleic acid encoding a guide RNA, and the nucleic acid encoding the selectable marker, wherein the nucleic acid encoding the Cas enzyme is maintained. In some embodiments, the nucleic acid encoding the selectable marker is removed by providing a site-specific recombinase to the cell. In some embodiments, the site-specific recombinase is provided to the cell by introducing protein or mRNA encoding the site-specific recombinase.

In some embodiments, the methods of present disclosure further comprise providing the nucleic acid encoding the Cas enzyme, the nucleic acid encoding the guide RNA, and the nucleic acid encoding the one or more selectable markers comprising transducing or transfecting the cell with one or more vectors comprising the nucleic acid encoding the guide RNA, and the nucleic acid encoding the selectable marker. In some embodiments, the methods disclosed above further comprise providing two or more vectors to the cell. In some embodiments, the two or more vectors comprises a selectable marker. In some embodiments, the two or more vectors comprises at least one selectable marker. In some embodiments, the two or more vectors comprise two selectable markers. In some embodiments, the selectable markers are different. In some embodiments, the selectable markers are flanked by recognition sites for the site-specific recombinase. In some embodiments, the nucleic acid encoding the Cas enzyme and the nucleic acid encoding the guide RNA are provided on two different vectors. In some embodiments, the method comprises selecting the cells for the selectable marker. In some embodiments, the selectable markers are removed following selecting the cells for the selectable marker. In some embodiments, selecting the cells for the selectable marker comprises culturing the cell with an antibiotic. In some embodiments, the cells are further cultured following selection with the selectable marker. In some embodiments, the nucleic acid encoding the guide RNA is not removed.

In some embodiments, the method of generating engineered cells comprises introducing a vector comprising the nucleic acid encoding Cas9 into a cell. In some embodiments, the vector is transduced into the cell using lentiviral transduction. Optimal transduction conditions may be determined by determining the functional titer of the lentiviral vectors. The number of viral particles used and the transduction efficiency will determine the average number of lentiviral integrations into the target genome. multiplicity of infection or MOI, is the number of infectious agents that enter the infection target and is the probability that an infection target (e.g., a cell) will get infected by infectious agents. Optimizing transduction conditions can extend the utility of viral particles and limit cell toxicity. In some embodiments, a MOI was used to transduce greater than 30% of the population of cells.

In some embodiments, the method of generating engineered cells comprises selecting a promoter to drive Cas expression. In some embodiments, the promoter is cloned into the MCS and operably linked to the Cas enzyme. In some embodiments, the Cas enzyme is Cas9. In some embodiments, the promoter is EF1a. In some embodiments, the promoter is human EF1a or murine EF1a. In some embodiments, the promoter driving Cas expression is selected by using a gene editing assay to measure Cas activity. In some embodiments, the assay comprises introducing a vector comprising a nucleic acid encoding a guide RNA targeting a detection marker. In some embodiments, the detection marker is encoded on the same nucleic acid as the guide RNA. In some embodiments, the detection marker is a destabilized EGFP. Cas activity may be expressed as percentages of GFP fluorescence, wherein the fluorescence of an edited population of cells is compared against a population of cells comprising a control (non-targeting) guide RNA. Therefore, GFP fluorescence may be monitored as a readout for gene editing. In some embodiments, the detection marker is an EGFP. Guide transduction and selection can be monitored as percentages of GFP fluorescent positive cells. Non-limiting methods used to assay fluorescence include FACS.

In some embodiments, the method of generating engineered cells comprises selecting cells which comprise a vector. In some embodiments, the method comprises selecting cells which comprise two vectors. In some embodiments, the method comprises providing the cell first and second vectors, wherein the first vector comprises the nucleic acid encoding the Cas enzyme and nucleic acid encoding a first selectable marker flanked by recognition sites for a site specific-recombinase, wherein the second vector comprises the nucleic acid encoding the guide RNA and nucleic acid encoding a second selectable marker flanked by recognition sites for a site-specific recombinase. The appropriate concentration of antibiotic for selecting stable cell lines is different for each cell type. If the concentration for the desired cell type is unknown, a titration experiment (e.g., a kill curve) must be performed to determine the lowest concentration of antibiotic needed to efficiently select transduced cells. Typically, 1-10 microgram per milliliter (μg/mL) antibiotic is sufficient to kill most untransduced mammalian cell types. In some embodiments, the antibiotic is blasticidin or puromycin. In some embodiments, the lowest concentration resulting in rapid (e.g., hours and days) killing of non-transduced cells was used. In some embodiments, the concentration of blasticidin is about 2 μg/mL to about 15 μg/mL. In some embodiments, the concentration of blasticidin is about 2.5 μg/mL. In some embodiments, the concentration of blasticidin is about 12.5 μg/mL. In some embodiments, the concentration of blasticidin is about 1 μg/mL to about 5 μg/mL. In some embodiments, the concentration of blasticidin used to select transductants is about 4 μg/mL. In some embodiments, the concentration of blasticidin used to select transductants is 1 μg/mL. In some embodiments, cells may be further maintained (e.g., cultured) in the presence of antibiotic to maintain selection pressure. In some embodiments, the antibiotic is blasticidin. In some embodiments, cells may be further maintained (e.g., cultured) in the presence of one or more antibiotics to maintain selection pressure. In some embodiments, the one or more antibiotics is blasticidin and/or puromycin.

In some embodiments, the method further comprises removing the selectable marker after any of the vectors as disclosed above has been introduced into the cell. In some embodiments, one or more selectable markers may be removed concurrently. In some embodiments, the selectable marker is removed using a site-specific recombinase. In some embodiments, the selectable marker is flanked by recognition sites. In some embodiments, the recognition sites are recognized by a site-specific recombinase and causes excision of the nucleic acid encoding the selectable marker. In some embodiments, the site-specific recombinase is Cre, Dre, or Flp. The selectable marker may be removed by the site-specific recombinase introduced into the cell. The site-specific recombinase may be introduced into the cell as a functional protein or as a nucleic acid. The site-specific recombinase may be introduced by various methods known to one of ordinary skill in the art. Non-limiting examples include proteofection, nanobodies, injection, and vesicle based delivery methods. In some embodiments, the site-specific recombinase is introduced as a nucleic acid. In some embodiments, the nucleic acid is an RNA. In some embodiments, the RNA is a mRNA. The mRNA may be introduced into the cell by methods well known to one of ordinary skill in the art including but not limited to transfection or transduction. Transfection can be carried out using calcium phosphate (i.e., tricalcium phosphate), by electroporation, by cell squeezing, or by mixing a cationic lipid with the material to produce liposomes that fuse with the cell membrane and deposit their cargo inside.

The mRNA may be made using methods known to one of ordinary skill in the art such as in vitro transcription. Briefly, a DNA template comprising a nucleic acid encoding a T7 promoter operably linked to the site-specific recombinase or a detection marker open reading frame, and a 120 polyA stretch may be transcribed into mRNA in vitro. The mRNA may be purified and capped using in vitro capping reactions. The cell is removed from selection media (e.g., media comprising antibiotics) prior to being contacted with mRNA template. In some embodiments, the mRNA template encoding the site-specific recombinase is introduced into the cell with the mRNA template encoding the detection marker to assess transfection efficiency. In some embodiments, the detection marker is mCherry or RFP.

In some embodiments, the one or more vectors comprises nucleic acid encoding a detection marker. In some embodiments, the nucleic acid encoding the detection marker is flanked by recognition sites for the site-specific recombinase. In some embodiments, the detection marker is a fluorescent protein. In some embodiments, the detection marker is GFP, YFP, RFP, EGFP, mCherry, or a luciferase. In some embodiments, the detection marker is a EGFP. In some embodiments, the detection marker is mCherry or RFP. In some embodiments, the method further comprises detecting the detection marker. In some embodiments, the detection marker is detected after six to eight days post transduction. In some embodiments, the detection marker is detected under antibiotic selection. Non-limiting examples of detecting the detection marker include microscopy or FACS.

In some embodiments, the method of generating engineered cells comprises contacting the cell with one or more vectors as disclosed above. In some embodiments, the vector comprises nucleic acids encoding a Cas enzyme. In some embodiments, the nucleic acid comprises a multiple cloning site upstream of a Cas enzyme. In some embodiments, the nucleic acid comprises, from 5′ to 3′, a 5′ LTR, a multiple cloning site, a Cas enzyme, a recognition site, an IRES site, a selection marker, a WPRE element, a recognition site, and a 3′ LTR. In some embodiments, a promoter is cloned into the multiple cloning site. In some embodiments, the nucleic acid comprises a promoter that is upstream and operably linked to a sequence encoding a Cas enzyme. In some embodiments, the nucleic acid comprises, from 5′ to 3′, 5′ LTR, a multiple cloning site, a sequence encoding a Cas enzyme, a recognition site, an IRES site, a selection marker, a WPRE element, another recognition site, and a 3′ LTR. In some embodiments, the promoter is a human CMV promoter, a human PGK promoter, a mouse PGK promoter, a human EF1a promoter, or a mouse EF1a promoter. In some embodiments, the Cas enzyme is a Cas9. In some embodiments, the recognition site is a loxP site. In some embodiments, the selection marker is the BSD gene (e.g., encoding resistance to blasticidin). In some embodiments, the selectable marker is removed upon expression of Cre.

In some embodiments, the method of generating engineered cells comprises contacting the cell with one or more vectors as disclosed above. In some embodiments, the vector comprises nucleic acids encoding a Cas enzyme. In some embodiments, the nucleic acid comprises a multiple cloning site upstream of a Cas enzyme. In some embodiments, the nucleic acid comprises, from 5′ to 3′, a 5′ LTR, a multiple cloning site, a Cas enzyme, a recognition site, an IRES site, a selectable marker, a WPRE element, a recognition site, and a 3′ LTR. In some embodiments, a promoter is cloned into the multiple cloning site. In some embodiments, the nucleic acid comprises a promoter that is upstream and operably linked to a sequence encoding a Cas enzyme. In some embodiments, the nucleic acid comprises, from 5′ to 3′, 5′ LTR, a multiple cloning site, a sequence encoding a Cas enzyme, a recognition site, an IRES site, a selectable marker, a WPRE element, another recognition site, and a 3′ LTR. In some embodiments, the promoter is a human CMV promoter, a human PGK promoter, a mouse PGK promoter, a human EF1a promoter, or a mouse EF1a promoter. In some embodiments, the Cas enzyme is a Cas9. In some embodiments, the recognition site is a loxP site. In some embodiments, the selectable marker is the BSD gene (e.g., encoding resistance to blasticidin). In some embodiments, the selectable marker is removed upon expression of Cre.

In some embodiments, the method of generating engineered cells contacting the cell with a vector comprising a nucleic acid that encodes a Cas enzyme and a selectable marker. In some embodiments, the method comprises contacting the cell with a vector comprising a nucleic acid that encodes a guide RNA and a selectable marker. In some embodiments, the method comprises contacting the cell with a vector comprising a nucleic acid that encodes the guide RNA, a selectable marker and a detection marker. In some embodiments, the method comprises contacting the cell with a vector comprising a nucleic acid that encodes a Cas enzyme, one or more guide RNAs, a selectable marker and a detection marker. In some embodiments, the method comprises contacting the cell with a vector comprising a nucleic acid that encodes wherein the vectors comprise one or more selectable markers, at least one detection marker, a Cas enzyme, and one or more guide RNAs.

In some embodiments, the method of generating engineered cells comprises contacting the cell with one or more vectors as disclosed above. In some embodiments, the vector comprises a nucleic acid that encodes a guide RNA. In some embodiments, the first guide RNA is the same as the second guide RNA. In some embodiments, the first guide RNA is different from the second guide RNA. In some embodiments, the first guide RNA targets the same gene as the second guide RNA. In some embodiments, the first guide RNA targets a different gene from the second guide RNA. In some embodiments, the detection marker is a fluorescent protein. In some embodiments, the detection marker is an EGFP. In some embodiments, the recognition site is a lox2772 site. In some embodiments, the selection marker is the PAC gene (e.g., encoding resistance to puromycin). In some embodiments, the selectable marker is removed upon expression of Cre. In some embodiments, the selectable marker is the PAC gene (e.g., encoding resistance to puromycin). In some embodiments, the selectable marker is removed upon expression of Cre.

In some embodiments, the method of generating engineered cells comprises contacting the cell with a vector (e.g., a first vector) and another vector (e.g., a second vector) as disclosed above. In some embodiments, the first vector comprises a nucleic acid that encodes a Cas enzyme. In some embodiments, the vector comprises from 5′ to 3′, a nucleic acid encoding Cas9, a recombination site for a site-specific recombinase, IRES, an antibiotic resistance (such as BSD or PAC) cassette, and a recombination site for a site-specific recombinase.

In some embodiments, the nucleic acid comprises a multiple cloning site upstream of a Cas enzyme. In some embodiments, the first guide RNA is the same as the second guide RNA. In some embodiments, the first guide RNA is different from the second guide RNA. In some embodiments, the first guide RNA targets the same gene as the second guide RNA. In some embodiments, the first guide RNA targets a different gene from the second guide RNA. In some embodiments, the detection marker is a fluorescent protein. In some embodiments, the detection marker is an EGFP. In some embodiments, the recognition site is a lox2772 site. In some embodiments, the selection marker is the PAC gene (e.g., encoding resistance to puromycin). In some embodiments, the selectable marker is the PAC gene (e.g., encoding resistance to puromycin). In some embodiments, both selectable markers are removed upon expression of Cre. In some embodiments, the selectable markers are the PAC gene and the BSD gene. In some expression of the nucleic acid encoding the Cas enzyme and the nucleic acid encoding the guide RNA results in editing in the genome of the cell. In some embodiments the vector comprises from 5′ to 3′ a promoter, a detection marker, a recombination site for a site-specific recombinase, an IRES, and an antibiotic resistance (such as PAC or BSD) cassette, a recombination site for a site-specific recombinase.

The methods disclosed in the present application may be applied to a cell that is a murine cell or a human cell. In some embodiments, the cell is a primary tumor cell or a cell from a tumor cell line. Some non-limiting examples of primary tumors include ones derived from the primary tumor is from a colon cancer, breast cancer, liver cancer, melanoma, B cell carcinoma. Some non-limiting examples of murine tumor cell lines include A20, B16F10, MC38, CT26, 4T1, and H22. In some embodiments, the cell generated by the methods of the present disclosure may be introduced into an organism. The mouse model of syngeneic tumor cell line transplantation can be constructed by subcutaneous injection (heterotopic) transplantation or orthotopic transplantation. Therefore, in some embodiments, the cell may implanted or injected into the organism. In some embodiments, the organism is tolerized to the Cas enzyme and/or GFP.

Methods of Introducing Cells into an Organism

The present disclosure provides for methods of introducing engineered cells comprising a Cas enzyme and a guide RNA into an organism that has an immune response, comprising generating the engineered cells by (a) providing one or more vectors comprising the Cas enzyme, the guide RNA, and a selectable marker to a plurality of cells, (b) culturing the cells such that the plurality of cells express the selectable marker, (c) isolating from the cultured cells, selected cells the express the selectable marker, (d) removing the selectable marker from the isolated, selected cells to produce engineered cells, (e) providing the engineered cells to the organism, wherein the organism is tolerized to the Cas enzyme. In some embodiments, the expression of Cas enzyme and/or the guide RNA are operably linked to an inducible promoter. In some embodiments, comprising activating the inducible promoter in the organism. In some embodiments, the nucleic acid encoding the Cas enzyme and/or the guide RNA are operably linked to a constitutive promoter. In some embodiments, the expression of the nucleic acid encoding the Cas enzyme and the nucleic acid encoding the guide RNA results in editing in the genome of the cell. In some embodiments, the organism is tolerized to the Cas enzyme. In some embodiments, tolerizing the organism to the Cas enzyme comprises expressing the Cas enzyme in the organism prior to providing the engineered cells to the organism. In some embodiments, the organism is a non-human animal. In some embodiments, the organism is a rat or a mouse. In some embodiments, the organism is a monkey, e.g., a cynomolgus monkey. In some embodiments, the organism is the same species as the engineered cell. In some embodiments, the organism is a different species from the cell. In some embodiments, the Cas enzyme is Cas9. In some embodiments, the cell is a mammalian cell. In some embodiments, the cell is a murine cell or a human cell. In some embodiments, the method further comprises culturing the cell prior to proving the cell to the organism. In some embodiments, the engineered cell is implanted or injected into the organism. In some embodiments, the organism is a mammal. In some embodiments, the organism is a rat or a mouse. In some embodiments, the organism is the same species at the cell. In some embodiments, the organism is a different species from the cell. In some embodiments, the cell does not express a selectable marker. In some embodiments, the cell comprising a vector as disclosed is selected for prior to introduction into an organism. In some embodiments, the cell comprising a vector as disclosed is selected for prior to introduction into an organism by a detection marker. In some embodiments, the cell is GFP+/Cas+. In some embodiments, the cell does not express a selection marker and is GFP+/Cas+. In some embodiments, the cell does not express a selectable marker and is GFP+/Cas+. In some embodiments, a rate of rejection may be measured after the cell is introduced into the organism. In some embodiments, the rate of rejection of the cell by the organism is reduced compared to a cell introduced into an organism which is not tolerized. In some embodiments, the rejection rate of the engineered cells by the organism is reduced by at least 20% compared to rate of rejection of the engineered cells introduced into an organism which is not tolerized. In some embodiments, the rejection rate of the engineered cells by the organism is reduced by at least 50% to rate of rejection of the engineered cells introduced into an organism which is not tolerized. In some embodiments, the rejection rate of the engineered cells by the organism is reduced by 20%-50% to rate of rejection of the engineered cells introduced into an organism which is not tolerized. In some embodiments, the engineered cells are tumor cells and the rate of tumor growth in the organism is increased compared to the rate of tumor growth of the engineered cells into an organism which is not tolerized. In some embodiments, the cell develops into a tumor in the organism. In some embodiments, the tumor is GFP+.

A mouse model is a laboratory mouse used to study some aspect of human physiology or disease. A variety of different model organisms are used in this regard, but mice are especially useful because they share mammalian features with humans and suffer from many of the same diseases. A large number of mouse models have been created to target specific human diseases using selective breeding and genetic engineering. Syngeneic models use tumor cells and transplantation host of a same inbred strain. Therefore, the host is unlikely to elicit an immune response against the tumor cells compared to a non-syngeneic host.

Tolerization of an organism is the prevention of an immune response against a particular antigen. For instance, the immune system is generally tolerant of self-antigens, so it does not usually attack the body's own cells, tissues, and organs. However, when tolerance is lost, disorders like autoimmune disease or food allergy may occur. Tolerance is maintained in a number of ways: immune tolerance, or immunological tolerance, or immunotolerance, is a state of unresponsiveness of the immune system to substances or tissue that would otherwise have the capacity to elicit an immune response in a given organism. It is induced by prior exposure to that specific antigen and contrasts with conventional immune-mediated elimination of foreign antigens. Appropriate reactivity toward certain antigens can also be quieted by induction of tolerance after repeated exposure, or exposure in a certain context. Immune tolerance contrasts with resistance. Immune tolerance is achieved under conditions that suppress the immune reaction; it is not just the absence of an immune response. The latter is a process of unresponsiveness to a specific antigen to which a person is normally responsive.

Self-tolerance is the immune system's ability to recognize what is ‘self’ and not react against or attack it. If immunological self-tolerance is lost, the body develops an autoimmunity against its own tissues and cells, which become the source of the autoimmune disease. Self-tolerance plays a key role in the prevention and treatment of immune disorder diseases, especially autoimmune diseases.

Tolerogenic therapy aims to induce immune tolerance where there is pathological or undesirable activation of the normal immune response. This can occur, for example, when an allogeneic transplantation patient develops an immune reaction to donor antigens, or when the body responds inappropriately to self-antigens implicated in autoimmune diseases. It must provide absence of specific antibodies for exactly that antigen. Upon exposure to a foreign antigen, either the antigen is eliminated by the standard immune response (resistance), or the immune system adapts to the pathogen, promoting immune tolerance instead. To develop tolerized organisms for models, the organism may be exposed to the antigen through many methods known to one of ordinary skill in the art, including but not limited to immunization, oral dosing, or creation of a knock-in mouse which expresses the antigen. Immune tolerance may be measured by assays known to one of ordinary skill in the art such as but not limited to: ELISA, Flow cytometry of intracellular cytokines, non-antigen specific assays for monitoring immunity and tolerance, phenotyping of immune recipient cells, and gene expression.

In some embodiments, the organism is tolerized against the Cas enzyme. In some embodiments, the organism is tolerized against Cas9. In some embodiments, the organism has a Cas enzyme in its germline DNA. In some embodiments, the organism expresses a Cas enzyme. In some embodiments, the organism is tolerized against EGFP. In some embodiments, the organism is tolerized against EGFP. In some embodiments, the organism has a EGFP in its germline DNA. In some embodiments, the organism expresses EGFP.

The cells are protected from rejection because the cells and the organism have the same genetic background. At the same time, the syngeneic tumor mouse model eliminates the complex process of immune reconstitution in immunodeficient mice. This advantage of maintaining a complete immune system in mice makes syngeneic tumor mouse models suitable for studying tumor microenvironment, tumor metastasis, and especially A powerful tool for evaluating immunomodulatory therapies. Therefore, the method of introducing cells into an organism results in tumor modulation (e.g., tumor growth). Tumor growth is thus also one measure of cell rejection by the organism. In some embodiments, cell rejection by the organism is reduced compared to a cell introduced into an organism which is not tolerized. In some embodiments, rejection is measured by tumor formation rate. In some embodiments, tumor growth is measured by tumor growth curves over time. In some embodiments, rejection is measured by slower tumor growth in an organism that is not tolerized compared to tumor growth in an organism that is tolerized.

The mouse model of syngeneic tumor cell line transplantation is to inoculate a histocompatibility tumor cell line, that is, a tumor cell line derived from the same background into immune-sound inbred mice. Compared with the CDX model, the mouse model of mouse tumor cell line transplantation retains the mouse's complete immune system and can be used to study the performance of cancer immunotherapy in the presence of a functional immune system.

The most commonly used host mice for traditional syngeneic transplantation models are Balb/c and C57BL/6 mice. Balb/c and C57BL/6 have some differences in immunology. For example, Balb/c has a stronger humoral response compared with C57BL/6 mice; in C57BL/6 mice, Th1 immune response and IFNγ production accounted for Dominance, and Balb/c easily triggers Th2 immune response, and so on. A variety of mouse tumor cell lines have been developed, mainly under the background of C57BL/6 and Balb/c. For example, the MC38 colon cancer cell line is derived from C57BL/6 inbred mice, and MC38 cells can be easily transplanted into C57BL/6 mice.

Syngeneic models are allografts immortalized from mouse cancer cell lines. For example, the MC38 tumorigenic epithelial cell line is isolated from mice with colon adenocarcinoma and expresses high levels of human carcinoembryonic antigen (CEA). B16-F10 is a cell line exhibiting a morphology of spindle-shaped and epithelial-like cells that was isolated from skin tissue of a mouse with melanoma. Other cell lines include, but are not limited to: 4T1 (Breast Cancer), CT26 (Colon Cancer), P338 (Leukemia), KLN 205 (Lung Cancer), A20 (Lymphoma), EL4 (Lymphoma), P815 (Mastocytoma) and Renca (Kidney Cancer).

Syngeneic models may be established in various genetic backgrounds. BALB/c is widely used in cancer immunotherapy research. Its sensitivity to carcinogens has made it the most imperative source for developing syngeneic tumor cell lines, such as, colon cancer (CT26), breast cancer (4T1), liver cancer (H22), melanoma (B16F10), and B cell carcinoma (A20). Similarly, C57BL/6, the most popular inbred strain in the scientific community, has been used extensively for making gene edited models for varied applications in genetics, oncology, and other studies. BALB/c and C57BL/6 have different immune characteristics, such as varying expression levels of CD14, toll-like receptor, TGF beta, and MDSC as well as different responses to some viral infections.

In some embodiments, a mouse may be tolerized prior to introducing the cell. Cells that may be used include but are not limited to known cells derived from primary tumors or tumor cell lines. In some embodiments, the mouse model may be a humanized mouse. Therefore, in some embodiments, a humanized mouse may be tolerized. A humanized mouse is a mouse carrying functioning human genes, cells, tissues, and/or organs. Humanized mice are commonly used as small animal models in biological and medical research for human therapeutics. A humanized mouse or a humanized mouse model is one that has been xenotransplanted with human cells and/or engineered to express human gene products, so as to be utilized for gaining relevant insights in the in vivo context for understanding of human-specific physiology and pathologies.

In some embodiments, the methods disclosed of introducing cells into an organism comprise generating a cell by contacting the cell with one or more vectors as disclosed above. In some embodiments, the vector comprises nucleic acids encoding a Cas enzyme. In some embodiments, the nucleic acid comprises a multiple cloning site upstream of a Cas enzyme. In some embodiments, the nucleic acid comprises, from 5′ to 3′, a 5′ LTR, a multiple cloning site, a Cas enzyme, a recognition site, an IRES site, a selection marker, a WPRE element, a recognition site, and a 3′ LTR. In some embodiments, the nucleic acid comprises, from 5′ to 3′, a 5′ LTR, a multiple cloning site, a Cas enzyme, a recognition site, an IRES site, a selection marker, a WPRE element, a recognition site, and a 3′ LTR. In some embodiments, the nucleic acid comprises, from 5′ to 3′, a 5′ LTR, a multiple cloning site, a Cas enzyme, a recognition site, an IRES site, a selectable marker, a WPRE element, a recognition site, and a 3′ LTR. In some embodiments, a promoter is cloned into the multiple cloning site. In some embodiments, the nucleic acid comprises a promoter that is upstream and operably linked to a sequence encoding a Cas enzyme. In some embodiments, the nucleic acid comprises, from 5′ to 3′, 5′ LTR, a multiple cloning site, a sequence encoding a Cas enzyme, a recognition site, an IRES site, a selection marker, a WPRE element, another Recognition site, and a 3′ LTR. In some embodiments, the nucleic acid comprises, from 5′ to 3′, 5′ LTR, a multiple cloning site, a sequence encoding a Cas enzyme, a recognition site, an IRES site, a selectable marker, a WPRE element, another Recognition site, and a 3′ LTR. In some embodiments, the promoter is a human CMV promoter, a human PGK promoter, a mouse PGK promoter, a human EF1a promoter, or a mouse EF1a promoter. In some embodiments, the promoter is hCMV, hPGK, mPGK, hEF1a, mEF1a. In some embodiments, the Cas enzyme is a Cas9. In some embodiments, the recognition site is a loxP site. In some embodiments, the selection marker is the BSD gene (e.g., encoding resistance to blasticidin). In some embodiments, the selection marker is removed upon expression of Cre. In some embodiments, the selectable marker is the BSD gene (e.g., encoding resistance to blasticidin). In some embodiments, the selectable marker is removed upon expression of Cre.

In some embodiments, the methods disclosed of introducing cells into an organism comprise generating a cell by contacting the cell with one or more vectors as disclosed above. In some embodiments, the vector comprises a nucleic acid that encodes a guide RNA. In some embodiments, the nucleic acid comprises, from 5′ to 3′, a PolIII promoter, a nucleic acid encoding a guide RNA, a detection marker, a recognition site, an IRES site, a selection marker, a WPRE element, and another recognition site. In some embodiments, the nucleic acid comprises, from 5′ to 3′, a PolIII promoter, a nucleic acid encoding a guide RNA, a detection marker, a recognition site, an IRES site, a selectable marker, a WPRE element, and another recognition site. In some embodiments, the PolIII promoter that is upstream and operably linked to a sequence encoding the guide RNA. Therefore in some embodiments, the nucleic acid comprises, from 5′ to 3′, a 5′ LTR, a PolIII promoter, a first guide RNA, a detection marker, a recognition site, an IRES site, a selection marker, a WPRE element, a recognition site, a PolIII promoter, a second guide RNA, and a 3′ LTR. n some embodiments, the nucleic acid comprises, from 5′ to 3′, a 5′ LTR, a PolIII promoter, a first guide RNA, a detection marker, a recognition site, an IRES site, a selectable marker, a WPRE element, a recognition site, a PolIII promoter, a second guide RNA, and a 3′ LTR. In some embodiments, the first guide RNA is the same as the second guide RNA. In some embodiments, the first guide RNA is different from the second guide RNA. In some embodiments, the first guide RNA targets the same gene as the second guide RNA. In some embodiments, the first guide RNA targets a different gene from the second guide RNA. In some embodiments, the detection marker is a fluorescent protein. In some embodiments, the detection marker is an EGFP. In some embodiments, the recognition site is a lox2772 site. In some embodiments, the selection marker is the PAC gene (e.g., encoding resistance to puromycin). In some embodiments, the selection marker is removed upon expression of Cre. In some embodiments, the selectable marker is the PAC gene (e.g., encoding resistance to puromycin). In some embodiments, the selectable marker is removed upon expression of Cre.

In some embodiments, the methods disclosed of introducing cells into an organism comprise generating a cell by contacting the cell with a vector (e.g., a first vector) and another vector (e.g., a second vector) as disclosed above. In some embodiments, the first vector comprises a nucleic acid that encodes a Cas enzyme. In some embodiments, the nucleic acid comprises a multiple cloning site upstream of a Cas enzyme. In some embodiments, the nucleic acid comprises, from 5′ to 3′, a 5′ LTR, a multiple cloning site, a Cas enzyme, a recognition site, an IRES site, a selection marker, a WPRE element, a recognition site, and a 3′ LTR. In some embodiments, the nucleic acid comprises, from 5′ to 3′, a 5′ LTR, a multiple cloning site, a Cas enzyme, a recognition site, an IRES site, a selectable marker, a WPRE element, a recognition site, and a 3′ LTR. In some embodiments, a promoter is cloned into the multiple cloning site. In some embodiments, the nucleic acid comprises a promoter that is upstream and operably linked to a sequence encoding a Cas enzyme. In some embodiments, the nucleic acid comprises, from 5′ to 3′, 5′ LTR, a multiple cloning site, a sequence encoding a Cas enzyme, a recognition site, an IRES site, a selection marker, a WPRE element, another recognition site, and a 3′ LTR. In some embodiments, the nucleic acid comprises, from 5′ to 3′, 5′ LTR, a multiple cloning site, a sequence encoding a Cas enzyme, a recognition site, an IRES site, a selectable marker, a WPRE element, another recognition site, and a 3′ LTR. In some embodiments, the promoter is a human CMV promoter, a human PGK promoter, a mouse PGK promoter, a human EF1a promoter, or a mouse EF1a promoter. In some embodiments, the promoter is hCMV, hPGK, mPGK, hEF1a, or mEF1a. In some embodiments, the Cas enzyme is a Cas9. In some embodiments, the recognition site is a loxP site. In some embodiments, the selection marker is the BSD gene (e.g., encoding resistance to blasticidin). In some embodiments, the selectable marker is the BSD gene (e.g., encoding resistance to blasticidin). In some embodiments, the second vector comprises a nucleic acid that encodes a guide RNA. In some embodiments, the nucleic acid comprises, from 5′ to 3′, a PolIII promoter, a nucleic acid encoding a guide, a detection marker, a recognition site, an IRES site, a selection marker, a WPRE element, and another recognition site. In some embodiments, the nucleic acid comprises, from 5′ to 3′, a PolIII promoter, a nucleic acid encoding a guide, a detection marker, a recognition site, an IRES site, a selection marker, a WPRE element, and another recognition site. Therefore in some embodiments, the nucleic acid comprises, from 5′ to 3′, a 5′ LTR, a PolIII promoter, a first guide RNA, a detection marker, a recognition site, an IRES site, a selection marker, a WPRE element, a recognition site, a PolIII promoter, a second guide RNA, and a 3′ LTR. In some embodiments, the nucleic acid comprises, from 5′ to 3′, a PolIII promoter, a nucleic acid encoding a guide, a detection marker, a recognition site, an IRES site, a selectable marker, a WPRE element, and another recognition site. In some embodiments, the PolIII promoter that is upstream and operably linked to a sequence encoding the guide RNA. Therefore in some embodiments, the nucleic acid comprises, from 5′ to 3′, a 5′ LTR, a PolIII promoter, a first guide RNA, a detection marker, a recognition site, an IRES site, a selectable marker, a WPRE element, a recognition site, a PolIII promoter, a second guide RNA, and a 3′ LTR. In some embodiments, the first guide RNA is the same as the second guide RNA. In some embodiments, the first guide RNA is different from the second guide RNA. In some embodiments, the first guide RNA targets the same gene as the second guide RNA. In some embodiments, the first guide RNA targets a different gene from the second guide RNA. In some embodiments, the detection marker is a fluorescent protein. In some embodiments, the detection marker is an EGFP. In some embodiments, the recognition site is a lox2772 site. In some embodiments, the selection marker is the PAC gene (e.g., encoding resistance to puromycin). In some embodiments, the selectable marker is the PAC gene (e.g., encoding resistance to puromycin). In some embodiments, both selectable markers are removed upon expression of Cre. In some embodiments, the selectable markers are the PAC gene and the BSD gene. In some expression of the nucleic acid encoding the Cas enzyme and the nucleic acid encoding the guide RNA results in editing in the genome of the cell.

In some embodiments, the method introduces one or more vectors into a cell that is a murine cell or a human cell. In some embodiments, the cell is a primary tumor cell or a cell from a tumor cell line. In some embodiments, the cell is a MC38 cell or a B16F10 cell. In some embodiments, the cell generated by the methods of the present disclosure may be introduced into an organism. In some embodiments, the cell may be introduced through implanted or injected into the organism. In some embodiments, the organism is tolerized to the Cas enzyme and/or GFP. In some embodiments, the organism is a mouse or a rat.

In some embodiments, introducing can be synonymous with administering. “Administering” refers to the physical introduction of a composition comprising an agent to a subject, using any of the various methods and delivery systems known to those skilled in the art. Routes of administration for cells comprising the vectors provided herein include injection and implantation Administering can also be performed, for example, once, a plurality of times, and/or over one or more extended periods.

### KITS

The present disclosure also provides kits comprising the vectors for use in generating the engineered cells described herein and introducing the engineered cell into an organism. The kit may comprise vectors, systems or engineered cells, cell lines, and reagents for introducing the modifications into the cells, such as the recombinase or plasmids for cloning. The kit may also comprise nucleic acids for expressing genes or cassettes described herein, or antibiotics for selection. The kit may also comprise instructions for making the engineered cell and introducing the engineered cell into a tolerized organism. The kit may also comprise reagents to tolerize an organism. Such a kit can further include instructions for making the desired modifications to host cells.

Kits typically include a label indicating the intended use of the contents of the kit and instructions for use. The term label includes any writing, or recorded material supplied on or with the kit, or which otherwise accompanies the kit.

In some embodiments, the kit comprises one or more vectors comprising nucleic acid encoding a Cas enzyme, nucleic acid encoding a guide RNA, and nucleic acid encoding a selectable marker wherein the nucleic acid encoding the selectable marker is flanked by recognition sequences for a site-specific recombinase. In some embodiments, the kit comprises a vector comprising a Cas enzyme and a selectable marker. In some embodiments, the kit comprises a vector comprising a guide RNA and a selectable marker. In some embodiments, the kit comprises a vector comprising the guide RNA, a selectable marker and a detection marker. In some embodiments, the kit comprises a vector comprising a Cas enzyme, one or more guide RNAs, a selectable marker and a detection marker. In some embodiments, the kit comprises two or more vectors, wherein the vectors comprise one or more selectable markers, at least one detection marker, a Cas enzyme, and one or more guide RNAs. In some embodiments, the kit comprises a vector comprising i) a nucleic acid encoding a Cas enzyme and/or or a nucleic acid encoding a guide RNA and ii) a nucleic acid encoding a selectable marker, wherein the nucleic acid encoding the selectable marker is flanked by recombination sites for a site-specific recombinase and wherein the nucleic acid encoding the Cas enzyme or the nucleic acid encoding the guide RNA is located outside of the sites for the site-specific recombinase. In some embodiments, the kit comprises a vector comprising i) a nucleic acid encoding a Cas enzyme and ii) a nucleic acid encoding a selectable marker, wherein the nucleic acid encoding the selectable marker is flanked by recombination sites for a site-specific recombinase and wherein the nucleic acid encoding the Cas enzyme or the nucleic acid encoding the guide RNA is located outside of the sites for the site-specific recombinase. In some embodiments, the vector comprises i) a nucleic acid encoding a nucleic acid encoding a guide RNA and ii) a nucleic acid encoding a selectable marker, wherein the nucleic acid encoding the selectable marker is flanked by recombination sites for a site-specific recombinase and wherein the nucleic acid encoding the Cas enzyme or the nucleic acid encoding the guide RNA is located outside of the sites for the site-specific recombinase. In some embodiments, the Cas is a Cas9. In some embodiments, the selectable marker is a BSD or a PAC. In some embodiments, the selectable markers are flanked by lox sites. In some embodiments, the selectable markers are removable by a site-specific recombinase. In some embodiments, the site-specific recombinase is Cre.

In some embodiments, the kit comprises a vector for expressing a site-specific recombinase. In some embodiments, the vector may be used for in vitro transcription. In some embodiments, the site-specific recombinase is Cre. In some embodiments, the kit comprises a vector for expressing a detection marker. In some embodiments, the detection marker is mCherry or RFP.

The instructions relating to the use of the vectors and reagents comprising such as described herein generally include information as to dosage, schedule, and method of introducing the vectors. The containers can be unit doses, bulk packages (e.g., multi-dose packages) or sub-unit doses. Instructions supplied in the kits of the disclosure are typically written instructions on a label or package insert.

The kits provided herein may be comprised within suitable packaging. Suitable packaging includes, but is not limited to, vials, bottles, jars, flexible packaging, and the like. Also contemplated are kits for use in combination with specific organism. In some embodiments, the kit comprises a non-human mammal. In some embodiments, the non-human mammal is a mouse. In some embodiments, the non-human mammal is syngeneic to an engineered cell comprising the vector made from the kit. Kits optionally can provide additional components such as buffers and interpretive information. Normally, the kit comprises a container and a label or package insert(s) on or associated with the container. In some embodiments, the disclosure provides instructions for use. comprising contents of the kits described above.

In some embodiments, the kit may be used on a non-human mammal. In some embodiments, the non-human mammal is a mouse. In some embodiments, the non-human mammal is syngeneic to an engineered cell comprising the vector made from the kit. Kits optionally can provide additional components as a needle for introducing an engineered cell into the non-human mammal. In some embodiments, the disclosure provides instructions for use, such as an administration schedule to tolerize a non-human mammal to an engineered cell comprising the vector.

### Exemplary Embodiments

Embodiment 1. A vector comprising i) a nucleic acid encoding a Cas enzyme and/or or a nucleic acid encoding a guide RNA and ii) a nucleic acid encoding a selectable marker, wherein the nucleic acid encoding the selectable marker is flanked by recognition sites for a site-specific recombinase and wherein the nucleic acid encoding the Cas enzyme or the nucleic acid encoding the guide RNA is located outside of the recognition sites for the site-specific recombinase.

Embodiment 2. The vector of Embodiment 1, wherein the vector comprises a nucleic acid encoding a Cas enzyme, wherein the Cas enzyme is a type I, type II, type III, type IV, or type V Cas enzyme.

Embodiment 3. The vector of Embodiment 2, wherein the Cas enzyme is a Cas9, a Cas10, or a Cas12.

Embodiment 4. The vector of any one of Embodiments 2-3, further comprising a promoter operably linked to the nucleic acid encoding the Cas enzyme and the selectable marker.

Embodiment 5. The vector of any one of Embodiments 1-4, wherein the vector comprises a nucleic acid encoding a guide RNA.

Embodiment 6. The vector of Embodiment 5, wherein the nucleic acid encoding the guide RNA is operably linked to a PolII promoter.

Embodiment 7. The vector of any one of Embodiments 1-6, wherein the selectable marker comprises an antibiotic resistance gene.

Embodiment 8. The vector of Embodiment 7, wherein the antibiotic resistance gene is BSD or PAC.

Embodiment 9. The vector of any one of Embodiments 4-8, wherein the promoter is an inducible promoter.

Embodiment 10. The vector of any one of Embodiments 4-9, wherein the promoter is a human or murine promoter.

Embodiment 11. The vector of any one of Embodiments 4-10, wherein the promoter is selected form the group consisting of hCMV, hPGK, mPGK, hEF1a, and mEF1a.

Embodiment 12. The vector of Embodiment 11, wherein the promoter is hEF1a.

Embodiment 13. The vector of any one of Embodiments 1-12, further comprising nucleic acid encoding a detection marker.

Embodiment 14. The vector of Embodiment 13, wherein the nucleic acid encoding the detection marker is flanked by recognition sites for the site-specific recombinase.

Embodiment 15. The vector of Embodiment 13, wherein the nucleic acid encoding the detection marker and the nucleic acid encoding the selection marker are flanked by the same recognition sites for the site-specific recombinase or wherein the nucleic acid encoding the detection marker and the selection marker are flanked by different recognition sites for a site-specific recombinase.

Embodiment 16. The vector of any one of Embodiments 13-15, wherein the detection marker is a fluorescent protein, optionally wherein the detection marker is GFP, YFP, RFP, EGFP, or a luciferase.

Embodiment 17. The vector of any one of Embodiments 1-16, comprising a nucleic acid encoding an internal ribosome entry site between the nucleic acid encoding the Cas enzyme and the selectable marker.

Embodiment 18. The vector of any one of Embodiments 1-17, wherein the site specific recombinase is selected from the group consisting of Cre, Dre, and Flp.

Embodiment 19. The vector of any one of Embodiments 1-18, wherein the vector is viral vector.

Embodiment 20. The vector of Embodiment 19, wherein the vector is an adenoviral or a lentiviral vector.

Embodiment 21. A system comprising the vector of any one of Embodiments 1-20.

Embodiment 22. The system of Embodiment 21, comprising a first vector comprising the nucleic acid encoding the Cas enzyme and a first selectable marker and a second vector comprising the nucleic acid encoding the guide RNA and a second selectable marker.

Embodiment 23. The system of Embodiment 22, wherein the first selectable marker and the second selectable marker are the different.

Embodiment 24. An engineered cell comprising the vector of any one of Embodiments 1-20 or the system of any one of Embodiments 21-23.

Embodiment 25. The engineered cell of Embodiment 24, wherein the cell is a human cell or a murine cell.

Embodiment 26. The engineered cell of any one of Embodiments 24-25, wherein the cell is a cell from a primary tumor or a tumor cell line.

Embodiment 27. A method of generating engineered cells comprising a nucleic acid encoding a Cas enzyme comprising
selecting a plurality of cells comprising a nucleic acid encoding a Cas enzyme, a nucleic acid encoding a guide RNA, and a nucleic acid encoding a selectable marker, and
removing from the selected cells the nucleic acid encoding the selectable, wherein the resulting engineered cells comprise the nucleic acid encoding the Cas enzyme and the nucleic acid encoding the guide RNA.

Embodiment 28. A method of generating cells comprising a nucleic acid encoding a Cas enzyme comprising removing nucleic acid encoding a selectable marker from a vector within a plurality of cells,
wherein the vector comprises a nucleic acid encoding a Cas enzyme, a nucleic acid encoding a guide RNA, and the nucleic acid encoding the selectable marker,
wherein the resulting cells comprise engineered cells comprising the nucleic acid encoding the Cas enzyme.

Embodiment 29. The method of Embodiment 27 or 28, wherein the nucleic acid encoding the selectable marker is removed by providing a site-specific recombinase to the plurality of cells.

Embodiment 30. The method of Embodiment 29, wherein the site-specific recombinase is selected from the group consisting of Cre, Dre, and Flp.

Embodiment 31. The method of Embodiment 29 or Embodiment 30, wherein providing the site-specific recombinase to the cell comprises providing protein or mRNA encoding the site-specific recombinase to the cell plurality of cells.

Embodiment 32. The method of Embodiment 31, further comprising performing in vitro transcription to produce the mRNA.

Embodiment 33. The method of any one of Embodiments 27-32, further comprising detecting removal of the nucleic acid encoding the selectable marker.

Embodiment 34. The method of Embodiment 30, wherein detecting removal of the selectable marker comprises assaying for nucleic acid encoding the selectable marker.

Embodiment 35. The method of any one of Embodiments 27-34, wherein prior to selecting or removing, the method comprises transducing or transfecting the cell plurality of cells with one or more vectors comprising the nucleic acid encoding the Cas enzyme, the nucleic acid encoding the guide RNA, and the nucleic acid encoding the selectable marker.

Embodiment 36. The method of any one of Embodiments 27-35, comprising providing two or more vectors to the cell, wherein at least one vector comprises the nucleic acid encoding the Cas enzyme, at least one vector comprises the nucleic acid encoding the guide RNA, and at least one vector comprises the nucleic acid encoding the selectable marker.

Embodiment 37. The method of Embodiment 36, wherein the two or more vectors comprises a selectable marker.

Embodiment 38. The method of Embodiment 36, wherein each of the selectable markers is flanked by recognition sites for the site-specific recombinase.

Embodiment 39. The method of Embodiment 37 or Embodiment 38, wherein the selectable marker comprises two or more distinct selection markers.

Embodiment 40. The method of any one of Embodiments 36-39, wherein the nucleic acid encoding the Cas enzyme and the nucleic acid encoding the guide RNA are provided on two different vectors.

Embodiment 41. The method of any one of Embodiments 36-40, comprising providing first and second vectors, wherein the first vector comprises the nucleic acid encoding the Cas enzyme and nucleic acid encoding a first selectable marker flanked by recognition sites for a site specific-recombinase and

wherein the second vector comprises the nucleic acid encoding the guide RNA and nucleic acid encoding a second selectable marker flanked by recognition sites for a site-specific recombinase.

Embodiment 42. The method of Embodiment 41, wherein the nucleic acid encoding the guide RNA and the nucleic acid encoding the Cas enzyme are each operably linked to a promoter.

Embodiment 43. The method of any one of Embodiments 28-42, wherein the selectable markers are removed following selecting the cells for the selectable marker.

Embodiment 44. The method of any one of Embodiments 28-43, further comprising selecting the cells for the selectable marker.

Embodiment 45. The method of Embodiment 27 or Embodiment 44, wherein selecting the cells for the selectable marker comprises culturing the cell with an antibiotic.

Embodiment 46. The method of Embodiment 44 or Embodiment 45, further comprising culturing the cell following selection with the selectable marker.

Embodiment 47. The method of any one of Embodiments 27-46, wherein the Cas enzyme is Cas9.

Embodiment 48. The method of any one of Embodiments 27-47, further comprising assaying the Cas enzyme activity in the engineered cells.

Embodiment 49. The method of any one of Embodiments 27-48, wherein the nucleic acid encoding the guide RNA is not removed.

Embodiment 50. The method of any one of Embodiments 27-49, wherein the one or more vectors comprises nucleic acid encoding a detection marker.

Embodiment 51. The method of Embodiment 50, wherein the nucleic acid encoding the detection marker is flanked by recognition sites for the site specific recombinase.

Embodiment 52. The method of Embodiment 50 or 51, wherein the detection marker is a fluorescent protein.

Embodiment 53. The method of any one of Embodiments 50-52, wherein the detection marker is GFP, YFP, RFP, EGFP, or a luciferase.

Embodiment 54. The method of any one of Embodiments 50-53, further comprising detecting the detection marker.

Embodiment 55. The method of any one of Embodiments 27-54, wherein the engineered cell is a primary tumor cell or a tumor cell line.

Embodiment 56. The method of any one of Embodiments 27-55, wherein the engineered cell is a murine cell or a human cell.

Embodiment 57. The method of any one of Embodiments 27-55, wherein the nucleic acid encoding the guide RNA is operably linked to a PolIII promoter.

Embodiment 58. The method of Embodiment 57, wherein the promoter is an inducible promoter.

Embodiment 59. The method of any one of Embodiments 27-58, wherein the nucleic acid encoding the Cas enzyme is operably linked to an inducible promoter.

Embodiment 60. The method of any one of Embodiments 57-59, wherein the promoter is a human promoter or a murine promoter.

Embodiment 61. The method of any one of Embodiments 27-60, comprising selecting cells which comprise a vector set forth in any one of Embodiments 1-20.

Embodiment 62. The method of any one of Embodiments 27-61, further comprising introducing the engineered cells into an organism.

Embodiment 63. The method of any one of Embodiments 27-62, wherein the organism is tolerized to the Cas enzyme and/or GFP.

Embodiment 64. The method of any one of Embodiments 27-63, wherein the engineered cells are engineered inducible cells that can be induced to express the Cas enzyme after introduction into an organism.

Embodiment 65. A method of introducing engineered cells comprising a Cas enzyme and a guide RNA into an organism that has an immune response, comprising
generating the engineered cells by providing one or more vectors comprising the Cas enzyme, the guide RNA, and a selectable marker to a plurality of cells,
culturing the cells such that the plurality of cells express the selectable marker,
isolating from the cultured cells, selected cells the express the selectable marker,
removing the selectable marker from the isolated, selected cells to produce engineered cells,
providing the engineered cells to the organism, wherein the organism is tolerized to the Cas enzyme.

Embodiment 66. The method of Embodiment 65, further comprising tolerizing the organism to the Cas enzyme.

Embodiment 67. The method of Embodiment 66, wherein tolerizing the organism to the Cas enzyme comprises expressing the Cas enzyme in the organism prior to providing the engineered cells to the organism.

Embodiment 68. The method of any one of Embodiments 65-67, wherein the engineered cell is implanted or injected into the organism.

Embodiment 69. The method of any one of Embodiments 65-68, further comprising culturing the engineered cells prior to providing the engineered cells to the organism.

Embodiment 70. The method of any one of Embodiments 65-69, wherein expression of Cas enzyme and/or the guide RNA are operably linked to an inducible promoter.

Embodiment 71. The method of Embodiment 70, further comprising activating the inducible promoter in the organism.

Embodiment 72. The method of any one of Embodiments 65-71, wherein expression of the nucleic acid encoding the Cas enzyme and the nucleic acid encoding the guide RNA results in editing in the genome of the engineered cell.

Embodiment 73. The method of any one of Embodiments 65-72, wherein the organism is a non-human mammal.

Embodiment 74. The method of Embodiment 73, wherein the organism is a rat or a mouse.

Embodiment 75. The method of any one of Embodiments 65-74, wherein the organism is the same species as the engineered cell.

Embodiment 76. The method of any one of Embodiments 65-75, wherein the organism is a different species from the cell.

Embodiment 77. The method of any one of Embodiments 65-76, wherein the Cas enzyme is a Cas9.

Embodiment 78. The method of any one of Embodiments 65-77, wherein the engineered cells are mammalian cells.

Embodiment 79. The method of any Embodiment 78, wherein the mammalian cells are murine cells or human cells.

Embodiment 80. The method of any one of Embodiments 65-79, wherein the engineered cells do not express a selectable marker.

Embodiment 81. The method of any one of Embodiments 65-80, comprising applying a selection for engineered cells which comprise a vector set forth in any one of Embodiments 1-20.

Embodiment 82. The method of any one of Embodiments 65-81, wherein the rate of rejection of the engineered cells by the organism is reduced compared to rate of rejection of the engineered cells introduced into an organism which is not tolerized.

Embodiment 83. The method of Embodiment 82, wherein the rejection rate of the engineered cells by the organism is reduced by at least 20% compared to rate of rejection of the engineered cells introduced into an organism which is not tolerized.

Embodiment 84. The method of Embodiment 82, wherein the rejection rate of the engineered cells by the organism is reduced by at least 50% to rate of rejection of the engineered cells introduced into an organism which is not tolerized.

Embodiment 85. The method of any one of Embodiments 65-84, wherein the engineered cells are tumor cells and the rate of tumor growth in the organism is increased compared to the rate of tumor growth of the engineered cells into an organism which is not tolerized.

Embodiment 86. A kit comprising the vector of any one of Embodiments 1-20, the system of any one of Embodiments 21-23 or the engineered cell of any one of Embodiments 24-26.

Embodiment 87. The kit of Embodiment 86, further comprising a non-human mammal.

Embodiment 88. The kit of Embodiment 86 or Embodiment 87, comprising instructions for use according to any one of Embodiments 65-85.

Embodiment 1A. A vector comprising i) a nucleic acid encoding a Cas enzyme and/or or a nucleic acid encoding a guide RNA and ii) a nucleic acid encoding a selectable marker, wherein the nucleic acid encoding the selectable marker is flanked by recognition sites for a site-specific recombinase and wherein the nucleic acid encoding the Cas enzyme or the nucleic acid encoding the guide RNA is located outside of the recognition sites for the site-specific recombinase.

Embodiment 2A. The vector of Embodiment 1A, wherein the vector comprises from 5′ to 3′ (i) the nucleic acid encoding the Cas enzyme, a first recognition site for the site specific recombinase, the nucleic acid encoding the selectable marker, and a second recognition site for the site specific recombinase; or
(ii) a first recognition site for the site specific recombinase, the nucleic acid encoding the selectable marker, a second recognition site for the site specific recombinase and the nucleic acid encoding the Cas enzyme.

Embodiment 3A. The vector of Embodiment 1A or 2A, wherein the vector comprises a nucleic acid encoding a Cas enzyme, wherein the Cas enzyme is a type I, type II, type III, type IV, or type V Cas enzyme.

Embodiment 4A. The vector of Embodiment 3, wherein the Cas enzyme is a Cas9, a Cas10, or a Cas12.

Embodiment 5A. The vector of Embodiment 3A or Embodiment 4A, further comprising a promoter operably linked to the nucleic acid encoding the Cas enzyme and the selectable marker.

Embodiment 6A. The vector of any one of Embodiments 1A-5A, wherein the vector comprises a nucleic acid encoding a guide RNA.

Embodiment 7A. The vector of Embodiment 1A, wherein the vector comprises from 5′ to 3′ (i) the nucleic acid encoding the guide RNA, a first recognition site for the site specific recombinase, the nucleic acid encoding the selectable marker, and a second recognition site for the site specific recombinase; or
(ii) a first recognition site for the site specific recombinase, the nucleic acid encoding the selectable marker, a second recognition site for the site specific recombinase and the nucleic acid encoding the guide RNA.

Embodiment 8A. The vector of Embodiment 6A or Embodiment 7A, wherein the nucleic acid encoding the guide RNA is operably linked to a PolIII promoter.

Embodiment 9A. The vector of any one of Embodiments 1A-8A, wherein the selectable marker comprises an antibiotic resistance gene.

Embodiment 10A. The vector of Embodiment 9A, wherein the antibiotic resistance gene is BSD or PAC.

Embodiment 11A. The vector of any one of Embodiments 5A-10A, wherein the promoter is an inducible promoter.

Embodiment 12A. The vector of any one of Embodiments 5A-11A, wherein the promoter is a human or murine promoter.

Embodiment 13A. The vector of any one of Embodiments 5A-12A, wherein the promoter is selected form the group consisting of hCMV, hPGK, mPGK, hEF1a, and mEF1a.

Embodiment 14A. The vector of Embodiment 13A, wherein the promoter is hEF1a.

Embodiment 15A. The vector of any one of Embodiments 1A-14A, further comprising a nucleic acid encoding a detection marker.

Embodiment 16A. The vector of Embodiment 15A, wherein the nucleic acid encoding the detection marker is flanked by recognition sites for the site-specific recombinase.

Embodiment 17A. The vector of Embodiment 15A, wherein the nucleic acid encoding the detection marker and the nucleic acid encoding the selectable marker are flanked by the same recognition sites for the site-specific recombinase or wherein the nucleic acid encoding the detection marker and the selectable marker are flanked by different recognition sites for a site-specific recombinase.

Embodiment 18A. The vector of Embodiment 15A, wherein the vector comprises from 5′ to 3′ (i) the nucleic acid encoding the detection marker, a first recognition site for the site specific recombinase, the nucleic acid encoding the selectable marker, a second recognition site for the site specific recombinase, and the nucleic acid encoding the Cas enzyme; (ii) the nucleic acid encoding the detection marker, a first recognition site for the site specific recombinase, the nucleic acid encoding the selectable marker, a second recognition site for the site specific recombinase, and the nucleic acid encoding the guide RNA; (iii) a first recognition site for the site specific recombinase, the nucleic acid encoding the detection marker, the nucleic acid encoding the selectable marker, a second recognition site for the site specific recombinase, and the nucleic acid encoding the Cas enzyme; or (iv) a first recognition site for the site specific recombinase, the nucleic acid encoding the detection marker, the nucleic acid encoding the selectable marker, a second recognition site for the site specific recombinase, and the nucleic acid encoding the guide RNA.

Embodiment 19A. The vector of any one of Embodiments 15A-17A, wherein the detection marker is a fluorescent protein, optionally wherein the detection marker is GFP, YFP, RFP, EGFP, or a luciferase.

Embodiment 20A. The vector of any one of Embodiments 1A-19A, comprising a nucleic acid encoding an internal ribosome entry site between the nucleic acid encoding the Cas enzyme and the selectable marker.

Embodiment 21A. The vector of any one of Embodiments 1A-20A, wherein the site specific recombinase is selected from the group consisting of Cre, Dre, and Flp.

Embodiment 22A. The vector of any one of Embodiments 1A-21A, wherein the vector is a viral vector.

Embodiment 23A. The vector of Embodiment 22A, wherein the vector is an adenoviral or a lentiviral vector.

Embodiment 24A. A system comprising the vector of any one of Embodiments 1A-23A.

Embodiment 25A. The system of Embodiment 24A, comprising a first vector comprising the nucleic acid encoding the Cas enzyme and a first selectable marker and a second vector comprising the nucleic acid encoding the guide RNA and a second selectable marker.

Embodiment 26A. The system of Embodiment 25A, wherein the first selectable marker and the second selectable marker are the different.

Embodiment 27A. An engineered cell comprising the vector of any one of Embodiments 1A-23A or the system of any one of Embodiments 24A-26A.

Embodiment 28A. The engineered cell of Embodiment 27A, wherein the cell is a human cell or a murine cell.

Embodiment 29A. The engineered cell of Embodiment 27A or Embodiment 28A, wherein the cell is a cell from a primary tumor or a tumor cell line.

Embodiment 30A. A method of generating engineered cells comprising a nucleic acid encoding a Cas enzyme comprising
selecting a plurality of cells comprising a nucleic acid encoding a Cas enzyme, a nucleic acid encoding a guide RNA, and a nucleic acid encoding a selectable marker, and
removing from the selected cells the nucleic acid encoding the selectable marker, wherein the resulting engineered cells comprise the nucleic acid encoding the Cas enzyme and the nucleic acid encoding the guide RNA.

Embodiment 31A. A method of generating cells comprising a nucleic acid encoding a Cas enzyme comprising removing the nucleic acid encoding a selectable marker from a vector within a plurality of cells, wherein the vector comprises a nucleic acid encoding a Cas enzyme, a nucleic acid encoding a guide RNA, and the nucleic acid encoding the selectable marker, wherein the resulting cells comprise engineered cells comprising the nucleic acid encoding the Cas enzyme.

Embodiment 32A. A method of generating engineered cells comprising a nucleic acid encoding a Cas enzyme and a nucleic acid encoding a guide RNA, the method comprising
(a) transducing the cells with a first vector comprising from 5′ to 3′ (i) a nucleic acid encoding a Cas enzyme, a first recognition site for a site specific recombinase, a nucleic acid encoding a selectable marker, and a second recognition site for a site specific recombinase; or (ii) a first recognition site for a site specific recombinase, a nucleic acid encoding a selectable marker, a second recognition site for a site specific recombinase and a nucleic acid encoding a Cas enzyme;
(b) transducing the cells with a second vector comprising from 5′ to 3′ (i) a nucleic acid encoding a guide RNA, a first recognition site for a site specific recombinase, a nucleic acid encoding a selectable marker, and a second recognition site for a site specific recombinase; or (ii) a first recognition site for a site specific recombinase, a nucleic acid encoding a selectable marker, a second recognition site for a site specific recombinase and a nucleic acid encoding guide RNA;
(c) selecting the cells comprising the nucleic acid encoding the selectable marker; and
(d) removing from the selected cells the nucleic acid encoding the selectable marker, wherein the resulting engineered cells comprise the nucleic acid encoding the Cas enzyme and the nucleic acid encoding the guide RNA.

Embodiment 33A. The method of any one of Embodiments 30A-32A, wherein the nucleic acid encoding the selectable marker is removed by providing a site-specific recombinase to the plurality of cells.

Embodiment 34A. The method of Embodiment 33A, wherein the site-specific recombinase is selected from the group consisting of Cre, Dre, and Flp.

Embodiment 35A. The method of Embodiment 33A or Embodiment 34A, wherein providing the site-specific recombinase to the cell comprises providing protein or mRNA encoding the site-specific recombinase to the cell plurality of cells.

Embodiment 36A. The method of Embodiment 35A, further comprising performing in vitro transcription to produce the mRNA.

Embodiment 37A. The method of any one of Embodiments 30A-36A, further comprising detecting removal of the nucleic acid encoding the selectable marker.

Embodiment 38A. The method of Embodiment 30A, wherein detecting removal of the selectable marker comprises assaying for nucleic acid encoding the selectable marker.

Embodiment 39A. The method of any one of Embodiments 30A-38A, wherein prior to selecting or removing, the method comprises transducing or transfecting the cell plurality of cells with one or more vectors comprising the nucleic acid encoding the Cas enzyme, the nucleic acid encoding the guide RNA, and the nucleic acid encoding the selectable marker.

Embodiment 40A. The method of any one of Embodiments 30A-39A, comprising providing two or more vectors to the cell, wherein at least one vector comprises the nucleic acid encoding the Cas enzyme, at least one vector comprises the nucleic acid encoding the guide RNA, and at least one vector comprises the nucleic acid encoding the selectable marker.

Embodiment 41A. The method of Embodiment 40A, wherein the two or more vectors comprises at least one selectable marker.

Embodiment 42A. The method of Embodiment 40A, wherein each of the selectable markers is flanked by recognition sites for the site-specific recombinase.

Embodiment 43A. The method of Embodiment 41A or Embodiment 42A, wherein the selectable marker comprises two or more distinct selectable marker.

Embodiment 44A. The method of any one of Embodiments 40A-43A, wherein the nucleic acid encoding the Cas enzyme and the nucleic acid encoding the guide RNA are provided on two different vectors.

Embodiment 45A. The method of any one of Embodiments 40A-44A, comprising providing first and second vectors, wherein the first vector comprises the nucleic acid encoding the Cas enzyme and a nucleic acid encoding a first selectable marker flanked by recognition sites for a site specific-recombinase and
wherein the second vector comprises the nucleic acid encoding the guide RNA and nucleic acid encoding a second selectable marker flanked by recognition sites for a site-specific recombinase.

Embodiment 46A. The method of Embodiment 45A, wherein the nucleic acid encoding the guide RNA and the nucleic acid encoding the Cas enzyme are each operably linked to a promoter.

Embodiment 47A. The method of any one of Embodiments 32A-46A, further comprising selecting the cells for the selectable markers.

Embodiment 48A. The method of any one of Embodiments 32A-47A, wherein the selectable markers are removed following selecting the cells for the selectable markers.

Embodiment 49A. The method of Embodiment 30A or 48A, wherein selecting the cells for the selectable marker comprises culturing the cell with an antibiotic.

Embodiment 50A. The method of Embodiment 48A or Embodiment 49A, further comprising culturing the cell following selection with the selectable marker.

Embodiment 51A. The method of any one of Embodiment 30A-50A, wherein the Cas enzyme is Cas9.

Embodiment 52A. The method of any one of Embodiments 30A-51A, further comprising assaying the Cas enzyme activity in the engineered cells.

Embodiment 53A. The method of any one of Embodiments 30A-52A, wherein the nucleic acid encoding the guide RNA is not removed.

Embodiment 54A. The method of any one of Embodiments 30A-53A, wherein the one or more vectors comprises a nucleic acid encoding a detection marker.

Embodiment 55A. The method of Embodiment 54A, wherein the nucleic acid encoding the detection marker is flanked by recognition sites for the site specific recombinase.

Embodiment 56A. The method of Embodiment 54A or 55A, wherein the detection marker is a fluorescent protein.

Embodiment 57A. The method of any one of Embodiments 54A-56A, wherein the detection marker is GFP, YFP, RFP, EGFP, or a luciferase.

Embodiment 58A. The method of any one of Embodiments 54A-57A, further comprising detecting the detection marker.

Embodiment 59A. The method of any one of Embodiments 30A-58A, wherein the engineered cell is a primary tumor cell or a tumor cell line.

Embodiment 60A. The method of any one of Embodiments 30A-59A, wherein the engineered cell is a murine cell or a human cell.

Embodiment 61A. The method of any one of Embodiments 30A-60A, wherein the nucleic acid encoding the guide RNA is operably linked to a PolIII promoter.

Embodiment 62A. The method of Embodiments 61A, wherein the promoter is an inducible promoter.

Embodiment 63A. The method of any one of Embodiments 30A-62A, wherein the nucleic acid encoding the Cas enzyme is operably linked to an inducible promoter.

Embodiment 64A. The method of any one of Embodiments 30A-63A, wherein the promoter is a human promoter or a murine promoter.

Embodiment 65A. The method of any one of Embodiments 30A-64A, comprising selecting cells which comprise a vector set forth in any one of Embodiments 1A-23A.

Embodiment 66A. The method of any one of Embodiments 30A-65A, further comprising introducing the engineered cells into an organism.

Embodiment 67A. The method of any one of Embodiments 30A-66A, wherein the organism is tolerized to the Cas enzyme and/or GFP.

Embodiment 68A. The method of any one of Embodiments 30A-67A, wherein the engineered cells are engineered inducible cells that can be induced to express the Cas enzyme after introduction into an organism.

Embodiment 69A. A method of introducing engineered cells comprising a Cas enzyme and a guide RNA into an organism that is immunocompetent, comprising
generating the engineered cells by
providing one or more vectors comprising the Cas enzyme, the guide RNA, and a selectable marker to a plurality of cells,
culturing the cells such that the plurality of cells express the selectable marker,
isolating from the cultured cells, selected cells the express the selectable marker,
removing the selectable marker from the isolated, selected cells to produce engineered cells,
providing the engineered cells to the organism, wherein the organism is tolerized to the Cas enzyme.

Embodiment 70A. The method of Embodiment 69A, further comprising tolerizing the organism to the Cas enzyme.

Embodiment 71A. The method of Embodiment 70A, wherein tolerizing the organism to the Cas enzyme comprises expressing the Cas enzyme in the organism prior to providing the engineered cells to the organism.

Embodiment 72A. The method of any one of Embodiment 69A-71A, wherein the engineered cells are implanted or injected into the organism.

Embodiment 73A. The method of any one of Embodiments 69A-72A, further comprising culturing the engineered cells prior to providing the engineered cells into the organism.

Embodiment 74A. The method of any one of Embodiments 69A-73A, wherein expression of Cas enzyme and/or the guide RNA are operably linked to an inducible promoter.

Embodiment 75A. The method of Embodiment 74A, further comprising activating the inducible promoter in the organism.

Embodiment 76A. The method of any one of Embodiment 73A-75A, wherein expression of the nucleic acid encoding the Cas enzyme and the nucleic acid encoding the guide RNA results in editing in the genome of the engineered cell.

Embodiment 77A. The method of any one of Embodiments 69A-76A, wherein the organism is a non-human mammal.

Embodiment 78A. The method of Embodiment 77A, wherein the organism is a rat or a mouse.

Embodiment 79A. The method of any one of Embodiments 69A-78A, wherein the organism is the same species as the engineered cell.

Embodiment 80A. The method of any one of Embodiments 69A-79A, wherein the organism is a different species from the cell.

Embodiment 81A. The method of any one of Embodiments 69A-80A, wherein the Cas enzyme is a Cas9.

Embodiment 82A. The method of any one of Embodiments 69A-81A, wherein the engineered cells are mammalian cells.

Embodiment 83A. The method of Embodiment 82A, wherein the mammalian cells are murine cells or human cells.

Embodiment 84A. The method of any one of Embodiments 69A-83A, wherein the engineered cells do not express a selectable marker.

Embodiment 85A. The method of any one of Embodiments 69A-84A, comprising applying a selection for engineered cells which comprise a vector set forth in any one of Embodiments 1A-23A.

Embodiment 86A. The method of any one of Embodiments 69A-85A, wherein the rate of rejection of the engineered cells by the organism is reduced compared to rate of rejection of the engineered cells introduced into an organism which is not tolerized.

Embodiment 87A. The method of Embodiment 86A, wherein the rejection rate of the engineered cells by the organism is reduced by at least 20% compared to rate of rejection of the engineered cells introduced into an organism which is not tolerized.

Embodiment 88A. The method of Embodiment 86A, wherein the rejection rate of the engineered cells by the organism is reduced by at least 50% to rate of rejection of the engineered cells introduced into an organism which is not tolerized.

Embodiment 89A. The method of any one of Embodiments 69A-88A, wherein the engineered cells are tumor cells and the rate of tumor growth in the organism is increased compared to the rate of tumor growth of the engineered cells into an organism which is not tolerized.

Embodiment 90A. A kit comprising the vector of any one of Embodiments 1A-23A, the system of any one of Embodiments 24A-26A or the engineered cell of any one of Embodiments 27A-29A.

Embodiment 91A. The kit of Embodiment 90A, further comprising a non-human mammal.

Embodiment 92A. The kit of Embodiment 90A or Embodiment 91A, comprising instructions for use according to any one of Embodiments 69A-89A.

### EXAMPLES

The following examples are included for illustrative purposes only and are not intended to limit the scope of the invention.

### Example 1: Generating Constructs with Removable Selection Markers and Cas9 Promoter Assessment

This example demonstrates a streamlined approach for in vitro selection marker removal from the CRISPR/Cas9 system in cells prior to implantation in mice. Selection marker removal was achieved by transient expression of Cre in the cells, and CRISPR/Cas9 system components were retained (FIG. 1A).

Materials and methods: In order to generate the construct, a base Cas9 vector was constructed with a bicistronic transcript cassette coding a FLAG-tagged Cas9 open reading frame (ORF; same as the Cas9-EGFP transgenic mice used) followed by a loxP flanked IRES-BSD (blasticidin resistance gene) (FIG. 1B). Different promoters as shown in Table 3 were tested and cloned into this construct (Table 4) following Esp3I (BsmBI) restriction digestion using Gibson Assembly with the following overlap sequences described in Table 5 appended via PCR.

TABLE 3
Promoter sequences tested in the
Cas9 constructs.

| SEQ | | |
|---|---|---|
| ID | | |
| NO: | Promoter | Sequence |
| 1 | hCMV | GACATTGATTATTGACTAGTTATTAATAGTAATCA |
| | | ATTACGGGGTCATTAGTTCATAGCCCATATATGGA |
| | | GTTCCGCGTTACATAACTTACGGTAAATGGCCCGC |
| | | CTGGCTGACCGCCCAACGACCCCCGCCCATTGACG |
| | | TCAATAATGACGTATGTTCCCATAGTAACGCCAAT |
| | | AGGGACTTTCCATTGACGTCAATGGGTGGAGTATT |
| | | TACGGTAAACTGCCCACTTGGCAGTACATCAAGTG |
| | | TATCATATGCCAAGTCCGCCCCCTATTGACGTCAA |
| | | TGACGGTAAATGGCCCGCCTGGCATTATGCCCAGT |
| | | ACATGACCTTACGGGACTTTCCTACTTGGCAGTAC |
| | | ATCTACGTATTAGTCATCGCTATTACCATGGTGAT |
| | | GCGGTTTTGGCAGTACACCAATGGGCGTGGATAGC |
| | | GGTTTGACTCACGGGGATTTCCAAGTCTCCACCCC |
| | | ATTGACGTCAATGGGAGTTTGTTTTGGCACCAAAA |
| | | TCAACGGGACTTTCCAAAATGTCGTAATAACCCCG |
| | | CCCCGTTGACGCAAATGGGCGGTAGGCGTGTACGG |
| | | TGGGAGGTCTATATAAGCAGAGCTGGTTTAGTGAA |
| | | CCGTCAGATCCGCTAGCGCTACCGGACTCAGATCT |
| | | CGAGCTCAAGCTTCGAATTCTGCAGTCGACGGTAC |
| | | CGCGGGCCCGGGATCCACCGGTC |
| 2 | hPGK | GGGGTTGGGGTTGCGCCTTTTCCAAGGCAGCCCTG |
| | | GGTTTGCGCAGGGACGCGGCTGCTCTGGGCGTGGT |
| | | TCCGGGAAACGCAGCGGCGCCGACCCTGGGTCTCG |
| | | CACATTCTTCACGTCCGTTCGCAGCGTCACCCGGA |
| | | TCTTCGCCGCTACCCTTGTGGGCCCCCCGGCGACG |
| | | CTTCCTGCTCCGCCCCTAAGTCGGGAAGGTTCCTT |
| | | GCGGTTCGCGGCGTGCCGGACGTGACAAACGGAAG |
| | | CCGCACGTCTCACTAGTACCCTCGCAGACGGACAG |
| | | CGCCAGGGAGCAATGGCAGCGCGCCGACCGCGATG |
| | | GGCTGTGGCCAATAGCGGCTGCTCAGCAGGGCGCG |
| | | CCGAGAGCAGCGGCCGGGAAGGGGCGGTGCGGGAG |
| | | GCGGGGTGTGGGGCGGTAGTGTGGGCCCTGTTCCT |
| | | GCCCGCGCGGTGTTCCGCATTCTGCAAGCCTCCGG |
| | | AGCGCACGTCGGCAGTCGGCTCCCTCGTTGACCGA |
| | | ATCACCGACCTCTCTCCCCAG |
| 3 | mPGK | GGGTAGGGGAGGCGCTTTTCCCAAGGCAGTCTGGA |
| | | GCATGCGCTTTAGCAGCCCCGCTGGGCACTTGGCG |
| | | CTACACAAGTGGCCTCTGGCCTCGCACACATTCCA |
| | | CATCCACCGGTAGGCGCCAACCGGCTCCGTTCTTT |
| | | GGTGGCCCCTTCGCGCCACCTTCTACTCCTCCCCT |
| | | AGTCAGGAAGTTCCCCCCCGCCCCGCAGCTCGCGT |
| | | CGTGCAGGACGTGACAAATGGAAGTAGCACGTCTC |
| | | ACTAGTCTCGTGCAGATGGACAGCACCGCTGAGCA |
| | | ATGGAAGCGGGTAGGCCTTTGGTGCAGCGGCCAAT |
| | | AGCAGCTTTGCTCCTTCGCTTTCTGGGCTCAGAGG |
| | | CTGGGAAGGGGTGGGTCCGGGGGCGGGCGCCCGAA |
| | | GGTCCTCCGGAGGCCCGGCATTCTGCACGCTTCAA |
| | | AAGCGCACGTCTGCCGCGCTGTTCTCCTCTTCCTC |
| | | ATCTCCGGGCCTTTCG |
| 4 | hEF1a | GGCTCCGGTGCCCGTCAGTGGGCAGAGCGCACATC |
| | | GCCCACAGTCCCCGAGAAGTTGGGGGGAGGGGTCG |
| | | GCAATTGAACCGGTGCCTAGAGAAGGTGGCGCGGG |
| | | GTAAACTGGGAAAGTGATGTCGTGTACTGGCTCCG |
| | | CCTTTTTCCCGAGGGTGGGGGAGAACCGTATATAA |
| | | GTGCAGTAGTCGCCGTGAACGTTCTTTTTCGCAAC |
| | | GGGTTTGCCGCCAGAACACAGGTAAGTGCCGTGTG |
| | | TGGTTCCCGCGGGCCTGGCCTCTTTACGGGTTATG |
| | | GCCCTTGCGTGCCTTGAATTACTTCCACCTGGCTG |
| | | CAGTACGTGATTCTTGATCCCGAGCTTCGGGTTGG |
| | | AAGTGGGTGGGAGAGTTCGAGGCCTTGCGCTTAAG |
| | | GAGCCCCTTCGCCTCGTGCTTGAGTTGAGGCCTGG |
| | | CCTGGGCGCTGGGGCCGCCGCGTGCGAATCTGGTG |
| | | GCACCTTCGCGCCTGTCTCGCTGCTTTCGATAAGT |
| | | CTCTAGCCATTTAAAATTTTTGATGACCTGCTGCG |
| | | ACGCTTTTTTTCTGGCAAGATAGTCTTGTAAATGC |
| | | GGGCCAAGATCTGCACACTGGTATTTCGGTTTTTG |
| | | GGGCCGCGGGCGGCGACGGGGCCCGTGCGTCCCAG |
| | | CGCACATGTTCGGCGAGGCGGGGCCTGCGAGCGCG |
| | | GCCACCGAGAATCGGACGGGGGTAGTCTCAAGCTG |
| | | GCCGGCCTGCTCTGGTGCCTGGCCTCGCGCCGCCG |
| | | TGTATCGCCCCGCCCTGGGCGGCAAGGCTGGCCCG |
| | | GTCGGCACCAGTTGCGTGAGCGGAAAGATGGCCGC |
| | | TTCCCGGCCCTGCTGCAGGGAGCTCAAAATGGAGG |
| | | ACGCGGCGCTCGGGAGAGCGGGCGGGTGAGTCACC |
| | | CACACAAAGGAAAAGGGCCTTTCCGTCCTCAGCCG |
| | | TCGCTTCATGTGACTCCACGGAGTACCGGGCGCCG |
| | | TCCAGGCACCTCGATTAGTTCTCGAGCTTTTGGAG |
| | | TACGTCGTCTTTAGGTTGGGGGGAGGGGTTTTATG |
| | | CGATGGAGTTTCCCCACACTGAGTGGGTGGAGACT |
| | | GAAGTTAGGCCAGCTTGGCACTTGATGTAATTCTC |
| | | CTTGGAATTTGCCCTTTTTGAGTTTGGATCTTGGT |
| | | TCATTCTCAAGCCTCAGACAGTGGTTCAAAGTTTT |
| | | TTTCTTCCATTTCAGGTGTCGTGA |
| 5 | mEF1a | AGAGTAATTCATACAAAAGGAGGGATCGCCTTCGC |
| | | CTGGGGAAGTCCCAGGGACCGTCGCTAAATTCTCA |
| | | TAACCCATAATCCCGGTACCCGCCCCACCACAGTG |
| | | CGAGGAGCATGCGCTCAGGGCTGAGCGCGGGGAGA |
| | | GCAGAGCACACAAGCTCATAGACCCTGGTCGTGGG |
| | | GGGAGGGGCGCACTGAGCGGGGGGGGGGGGGGTGA |
| | | TGGGGGGGAGGACCGGGGAGCTGGCGCGGGGCAAA |
| | | CTGGGAAAGCGGTGTCGTGTGCTGGCTCCGCCCTC |
| | | TTCCCGAGGGTGGGGGAGAACGGTATATAAGTGCG |
| | | GCAGTCGCCTTGGACGTTCTTTTTCGCAACGGGTT |
| | | TGCCGTCAGAACGCAGGTGAGGGGCGGGTGTGGCT |
| | | TCCGCGGGCCGCCGAGCTGGAGGTCCTGCTCCGAG |
| | | CGGGCCGGGCCCCGCTGTCGTCGGCGGGGATTAGC |
| | | TGCGAGCATTCCCGCTTCGAGTTGCGGGCGGCGCG |
| | | GGAGGCAGAGTGCGAGGCCTAGCGGCAACCCCGTA |
| | | GCCTCGCCTCGTGTCCGGCTTGAGGCCTAGCGTGG |
| | | TGTCCGCGCCGCCGCCGCGTGCTACTCCGGCCGCA |
| | | CTCTGGTCTTTTTTTTTTTGTTGTTGTTGTTGCCC |
| | | TGCTGCCTTCGATTGCCGTTCAGCAATAGGGGCTA |
| | | ACAAAGGGAGGGTGCGGGGCTTGCTCGCCCGGAGC |
| | | CCGGAGAGGTCATGGTTGGGGAGGAATGGAGGGAC |
| | | AGGAGTGGCGGCTGGGGCCCGCCCGCCTTCGGAGC |
| | | ACATGTCCGACGCCACCTGGATGGGGCGAGGCCTG |
| | | GGGTTTTTCCCGAAGCAACCAGGCTGGGGTTAGCG |
| | | TGCCGAGGCCATGTGGCCCCAGCACCCGGCACGAT |
| | | CTGGCTTGGCGGCGCCGCGTTGCCCTGCCTCCCTA |
| | | ACTAGGGTGAGGCCATCCCGTCCGGCACCAGTTGC |
| | | GTGCGTGGAAAGATGGCCGCTCCCGGGCCCTGTTG |
| | | CAAGGAGCTCAAAATGGAGGACGCGGCAGCCCGGT |
| | | GGAGCGGGCGGGTGAGTCACCCACACAAAGGAAGA |
| | | GGGCCTGGTCCCTCACCGGCTGCTGCTTCCTGTGA |
| | | CCCCGTGGTCCTATCGGCCGCAATAGTCACCTCGG |
| | | GCTTTTGAGCACGGCTAGTCGCGGCGGGGGGAGGG |
| | | GATGTAATGGCGTTGGAGTTTGTTCACATTTGGTG |
| | | GGTGGAGACTAGTCAGGCCAGCCTGGCGCTGGAAG |
| | | TCATTTTTGGAATTTGTCCCCTTGAGTTTTGAGCG |
| | | GAGCTAATTCTCGGGCTTCTTAGCGGTTCAAAGGT |
| | | ATCTTTTAAACCCTTTTTTAGGTGTTGTGAAAACC |
| | | ACCGCTAATTCAAAGCAA |

TABLE 4
Elements in the Casy constructs.

| SEQ | | |
|---|---|---|
| ID NO: | Promoter | Sequence |
| 6 | Flag- | GGATCATGATATTGATTACAAAGACGATGA |
| | Cas9 | CGATAAGATGGCCCATGGTGAGCAAGGGCG |
| | | ACTATAAGGACCACGACGGAGACTACAACA |
| | | AAGAAGAAGCGGAAGGTCGGTATCCACGGA |
| | | GTCCCAGCAGCCGACAAGAAGTACAGCATC |
| | | GGCCTGGACATCGGCACCAACTCTGTGGGC |
| | | TGGGCCGTGATCACCGACGAGTACAAGGTG |
| | | CCCAGCAAGAAATTCAAGGTGCTGGGCAAC |
| | | ACCGACCGGCACAGCATCAAGAAGAACCTG |
| | | ATCGGAGCCCTGCTGTTCGACAGCGGCGAA |
| | | ACAGCCGAGGCCACCCGGCTGAAGAGAACC |
| | | GCCAGAAGAAGATACACCAGACGGAAGAAC |
| | | CGGATCTGCTATCTGCAAGAGATCTTCAGC |
| | | AACGAGATGGCCAAGGTGGACGACAGCTTC |
| | | TTCCACAGACTGGAAGAGTCCTTCCTGGTG |
| | | GAAGAGGATAAGAAGCACGAGCGGCACCCC |
| | | ATCTTCGGCAACATCGTGGACGAGGTGGCC |
| | | TACCACGAGAAGTACCCCACCATCTACCAC |
| | | CTGAGAAAGAAACTGGTGGACAGCACCGAC |
| | | AAGGCCGACCTGCGGCTGATCTATCTGGCC |
| | | CTGGCCCACATGATCAAGTTCCGGGGCCAC |
| | | TTCCTGATCGAGGGCGACCTGAACCCCGAC |
| | | AACAGCGACGTGGACAAGCTGTTCATCCAG |
| | | CTGGTGCAGACCTACAACCAGCTGTTCGAG |
| | | GAAAACCCCATCAACGCCAGCGGCGTGGAC |
| | | GCCAAGGCCATCCTGTCTGCCAGACTGAGC |
| | | AAGAGCAGACGGCTGGAAAATCTGATCGCC |
| | | CAGCTGCCCGGCGAGAAGAAGAATGGCCTG |
| | | TTCGGAAACCTGATTGCCCTGAGCCTGGGC |
| | | CTGACCCCCAACTTCAAGAGCAACTTCGAC |
| | | CTGGCCGAGGATGCCAAACTGCAGCTGAGC |
| | | AAGGACACCTACGACGACGACCTGGACAAC |
| | | CTGCTGGCCCAGATCGGCGACCAGTACGCC |
| | | GACCTGTTTCTGGCCGCCAAGAACCTGTCC |
| | | GACGCCATCCTGCTGAGCGACATCCTGAGA |
| | | GTGAACACCGAGATCACCAAGGCCCCCCTG |
| | | AGCGCCTCTATGATCAAGAGATACGACGAG |
| | | CACCACCAGGACCTGACCCTGCTGAAAGCT |
| | | CTCGTGCGGCAGCAGCTGCCTGAGAAGTAC |
| | | AAAGAGATTTTCTTCGACCAGAGCAAGAAC |
| | | GGCTACGCCGGCTACATTGACGGCGGAGCC |
| | | AGCCAGGAAGAGTTCTACAAGTTCATCAAG |
| | | CCCATCCTGGAAAAGATGGACGGCACCGAG |
| | | GAACTGCTCGTGAAGCTGAACAGAGAGGAC |
| | | CTGCTGCGGAAGCAGCGGACCTTCGACAAC |
| | | GGCAGCATCCCCCACCAGATCCACCTGGGA |
| | | GAGCTGCACGCCATTCTGCGGCGGCAGGAA |
| | | GATTTTTACCCATTCCTGAAGGACAACCGG |
| | | GAAAAGATCGAGAAGATCCTGACCTTCCGC |
| | | ATCCCCTACTACGTGGGCCCTCTGGCCAGG |
| | | GGAAACAGCAGATTCGCCTGGATGACCAGA |
| | | AAGAGCGAGGAAACCATCACCCCCTGGAAC |
| | | TTCGAGGAAGTGGTGGACAAGGGCGCTTCC |
| | | GCCCAGAGCTTCATCGAGCGGATGACCAAC |
| | | TTCGATAAGAACCTGCCCAACGAGAAGGTG |
| | | CTGCCCAAGCACAGCCTGCTGTACGAGTAC |
| | | TTCACCGTGTATAACGAGCTGACCAAAGTG |
| | | AAATACGTGACCGAGGGAATGAGAAAGCCC |
| | | GCCTTCCTGAGCGGCGAGCAGAAAAAGGCC |
| | | ATCGTGGACCTGCTGTTCAAGACCAACCGG |
| | | AAAGTGACCGTGAAGCAGCTGAAAGAGGAC |
| | | TACTTCAAGAAAATCGAGTGCTTCGACTCC |
| | | GTGGAAATCTCCGGCGTGGAAGATCGGTTC |
| | | AACGCCTCCCTGGGCACATACCACGATCTG |
| | | CTGAAAATTATCAAGGACAAGGACTTCCTG |
| | | GACAATGAGGAAAACGAGGACATTCTGGAA |
| | | GATATCGTGCTGACCCTGACACTGTTTGAG |
| | | GACAGAGAGATGATCGAGGAACGGCTGAAA |
| | | ACCTATGCCCACCTGTTCGACGACAAAGTG |
| | | ATGAAGCAGCTGAAGCGGCGGAGATACACC |
| | | GGCTGGGGCAGGCTGAGCCGGAAGCTGATC |
| | | AACGGCATCCGGGACAAGCAGTCCGGCAAG |
| | | ACAATCCTGGATTTCCTGAAGTCCGACGGC |
| | | TTCGCCAACAGAAACTTCATGCAGCTGATC |
| | | CACGACGACAGCCTGACCTTTAAAGAGGAC |
| | | ATCCAGAAAGCCCAGGTGTCCGGCCAGGGC |
| | | GATAGCCTGCACGAGCACATTGCCAATCTG |
| | | GCCGGCAGCCCCGCCATTAAGAAGGGCATC |
| | | CTGCAGACAGTGAAGGTGGTGGACGAGCTC |
| | | GTGAAAGTGATGGGCCGGCACAAGCCCGAG |
| | | AACATCGTGATCGAAATGGCCAGAGAGAAC |
| | | CAGACCACCCAGAAGGGACAGAAGAACAGC |
| | | CGCGAGAGAATGAAGCGGATCGAAGAGGGC |
| | | ATCAAAGAGCTGGGCAGCCAGATCCTGAAA |
| | | GAACACCCCGTGGAAAACACCCAGCTGCAG |
| | | AACGAGAAGCTGTACCTGTACTACCTGCAG |
| | | AATGGGCGGGATATGTACGTGGACCAGGAA |
| | | CTGGACATCAACCGGCTGTCCGACTACGAT |
| | | GTGGACCATATCGTGCCTCAGAGCTTTCTG |
| | | AAGGACGACTCCATCGACAACAAGGTGCTG |
| | | ACCAGAAGCGACAAGAACCGGGGCAAGAGC |
| | | GACAACGTGCCCTCCGAAGAGGTCGTGAAG |
| | | AAGATGAAGAACTACTGGCGGCAGCTGCTG |
| | | AACGCCAAGCTGATTACCCAGAGAAAGTTC |
| | | GACAATCTGACCAAGGCCGAGAGAGGCGGC |
| | | CTGAGCGAACTGGATAAGGCCGGCTTCATC |
| | | AAGAGACAGCTGGTGGAAACCCGGCAGATC |
| | | ACAAAGCACGTGGCACAGATCCTGGACTCC |
| | | CGGATGAACACTAAGTACGACGAGAATGAC |
| | | AAGCTGATCCGGGAAGTGAAAGTGATCACC |
| | | CTGAAGTCCAAGCTGGTGTCCGATTTCCGG |
| | | AAGGATTTCCAGTTTTACAAAGTGCGCGAG |
| | | ATCAACAACTACCACCACGCCCACGACGCC |
| | | TACCTGAACGCCGTCGTGGGAACCGCCCTG |
| | | ATCAAAAAGTACCCTAAGCTGGAAAGCGAG |
| | | TTCGTGTACGGCGACTACAAGGTGTACGAC |
| | | GTGCGGAAGATGATCGCCAAGAGCGAGCAG |
| | | GAAATCGGCAAGGCTACCGCCAAGTACTTC |
| | | TTCTACAGCAACATCATGAACTTTTTCAAG |
| | | ACCGAGATTACCCTGGCCAACGGCGAGATC |
| | | CGGAAGCGGCCTCTGATCGAGACAAACGGC |
| | | GAAACCGGGGAGATCGTGTGGGATAAGGGC |
| | | CGGGATTTTGCCACCGTGCGGAAAGTGCTG |
| | | AGCATGCCCCAAGTGAATATCGTGAAAAAG |
| | | ACCGAGGTGCAGACAGGCGGCTTCAGCAAA |
| | | GAGTCTATCCTGCCCAAGAGGAACAGCGAT |
| | | AAGCTGATCGCCAGAAAGAAGGACTGGGAC |
| | | CCTAAGAAGTACGGCGGCTTCGACAGCCCC |
| | | ACCGTGGCCTATTCTGTGCTGGTGGTGGCC |
| | | AAAGTGGAAAAGGGCAAGTCCAAGAAACTG |
| | | AAGAGTGTGAAAGAGCTGCTGGGGATCACC |
| | | ATCATGGAAAGAAGCAGCTTCGAGAAGAAT |
| | | CCCATCGACTTTCTGGAAGCCAAGGGCTAC |
| | | AAAGAAGTGAAAAAGGACCTGATCATCAAG |
| | | CTGCCTAAGTACTCCCTGTTCGAGCTGGAA |
| | | AACGGCCGGAAGAGAATGCTGGCCTCTGCC |
| | | GGCGAACTGCAGAAGGGAAACGAACTGGCC |
| | | CTGCCCTCCAAATATGTGAACTTCCTGTAC |
| | | CTGGCCAGCCACTATGAGAAGCTGAAGGGC |
| | | TCCCCCGAGGATAATGAGCAGAAACAGCTG |
| | | TTTGTGGAACAGCACAAGCACTACCTGGAC |
| | | GAGATCATCGAGCAGATCAGCGAGTTCTCC |
| | | AAGAGAGTGATCCTGGCCGACGCTAATCTG |
| | | GACAAAGTGCTGTCCGCCTACAACAAGCAC |
| | | CGGGATAAGCCCATCAGAGAGCAGGCCGAG |
| | | AATATCATCCACCTGTTTACCCTGACCAAT |
| | | CTGGGAGCCCCTGCCGCCTTCAAGTACTTT |
| | | GACACCACCATCGACCGGAAGAGGTACACC |
| | | AGCACCAAAGAGGTGCTGGACGCCACCCTG |
| | | ATCCACCAGAGCATCACCGGCCTGTACGAG |
| | | ACACGGATCGACCTGTCTCAGCTGGGAGGC |
| | | GACAAAAGGCCGGCGGCCACGAAAAAGGCC |
| | | GGCCAGGCAAAAAAGAAAAAGTAA |
| 7 | LoxP | ATAACTTCGTATAGCATACATTATACGAAG |
| | | TTAT |
| 8 | IRES | tccctcccccccccctaacgttactggccg |
| | | aagccgcttggaataaggccggtgtgcgtt |
| | | tgtctatatgttattttccaccatattgcc |
| | | gtcttttggcaatgtgagggcccggaaacc |
| | | tggccctgtcttcttgacgagcattcctag |
| | | gggtctttcccctctcgccaaaggaatgca |
| | | aggtctgttgaatgtcgtgaaggaagcagt |
| | | tcctctggaagcttcttgaagacaaacaac |
| | | gtctgtagcgaccctttgcaggcagcggaa |
| | | ccccccacctggcgacaggtgcctctgcgg |
| | | ccaaaagccacgtgtataagatacacctgc |
| | | aaaggcggcacaaccccagtgccacgttgt |
| | | gagttggatagttgtggaaagagtcaaatg |
| | | gctctcctcaagcgtattcaacaaggggct |
| | | gaaggatgcccagaaggtaccccattgtat |
| | | gggatctgatctggggcctcggtgcacatg |
| | | ctttacgtgtgtttagtcgaggttaaaaaa |
| | | acgtctaggccccccgaaccacggggacgt |
| | | ggttttcctttgaaaaacacgatgataat |
| 9 | BSD | ATGGCCAAGCCTTTGTCTCAAGAAGAATCC |
| | | ACCCTCATTGAAAGAGCAACGGCTACAATC |
| | | AACAGCATCCCCATCTCTGAAGACTACAGC |
| | | GTCGCCAGCGCAGCTCTCTCTAGCGACGGC |
| | | CGCATCTTCACTGGTGTCAATGTATATCAT |
| | | TTTACTGGGGGACCTTGTGCAGAACTCGTG |
| | | GTGCTGGGCACTGCTGCTGCTGCGGCAGCT |
| | | GGCAACCTGACTTGTATCGTCGCGATCGGA |
| | | AATGAGAACAGGGGCATCTTGAGCCCCTGC |
| | | GGACGGTGCCGACAGGTGCTTCTCGATCTG |
| | | CATCCTGGGATCAAAGCCATAGTGAAGGAC |
| | | AGTGATGGACAGCCGACGGCAGTTGGGATT |
| | | CGTGAATTGCTGCCCTCTGGTTATGTGTGG |
| | | GAGGGCCTGCAGCTGCAGTAG |

TABLE 5
Promoter amplicon primer sequences

| SEQ | | | |
|---|---|---|---|
| ID | | | |
| NO: | Promoter | Sequence | |
| 10 | Upstream | TTCCGCG | Added to 5′ |
| | amplicon | GCTCTTCC | end of the |
| | primer | | upstream |
| | | | PCR primer |
| 11 | Downstream | GCCCTTG | Added to the |
| | amplicon | CTCACCA | 5′ end of the |
| | primer | TGGTGGC | downstream |
| | | | PCR primer) |

Cas9 constructs with various promoters were used to make lentivirus expression vectors. Lentivirus was produced per a standard protocol. Syngeneic mouse cell lines (either the MC38 cell line, B16F10 cell line, or KP2 cell line) were transduced with a target multiplicity of infection (MOI) of approximately 30%, followed by contact with blasticidin to select cells carrying the Cas9 vector.

Optimal selection concentrations for antibiotics (i.e., blasticidin and puromycin) were determined for each cell line using a kill curve. The lowest concentration resulting in rapid (e.g., days) killing of non-transduced cells was used (Table 6).

TABLE 6
Antibiotic titration for selection of transduced cells.

| | | Concentration |
|---|---|---|
| Cell line | Antibiotic | (μg/ml) |
| MC38 | Blasticidin | 2.5 |
| MC38 | Puromycin | 4 |
| B16F10 | Blasticidin | 12.5 |
| B16F10 | Puromycin | 1 |
| KP2 | Blasticidin | 4 |
| KP2 | Puromycin | 4 |

To identify promoters that yielded high Cas9 activity, aliquots of Cas9 cells were transduced in parallel with either negative control constructs or guide constructs targeting the EGFP locus in the guide construct (vector) to assay for Cas9 activity (both constructs constitutively express EGFP) (FIG. 1A). Puromycin was used as the selectable marker on the guide vector, and puromycin selection was performed identically to the above blasticidin selection, for 6-8 days (to allow for Cas9 cutting of EGFP, and loss of EGFP protein) then analyzed for EGFP expression. EGFP expression was measured by flow cytometry analysis. Blasticidin selection was maintained throughout puromycin selection.

Results: Data show that the human EF1a promoter (hEF1a) showed the highest amount of Cas9 activity in the MC38 cells. In B16F10 cells, the mouse EF1a (mEF1a) promoter showed the greatest amount of Cas9 activity. As a negative control, all transduced cells were observed to be EGFP+ (EGFP⁻ cells indicated incomplete selection). Cas9 activity was determined by producing self-targeting transduced cells by using a guide RNA against EGFP and subsequently analyzing the percent of EGFP− cells (Table 7). The lines with the highest Cas9 activity (hEF1a driven Cas9 for MC38; mEF1a driven Cas9 for B16F10) were carried forward for further engineering.

TABLE 7
Cas9 activity of various promoters in different cell lines.

| Cell line | Promoter | Cas9 Activity (%) |
|---|---|---|
| MC38 | hCMV | 86 |
| MC38 | hPGK | 38 |
| MC38 | hEF1a | 96 |
| MC38 | mEF1a | 96 |
| B16F10 | mEF1a | 88 |
| KP2 | hEF1a | 91 |

### Example 2: Guide Construct Transduction in Syngeneic Models

This example analyzed whether the guide constructs could be efficiently introduced into syngeneic cells. A base guide vector was cloned and efficiently introduced into syngeneic cells that were already carrying the Cas9 construct as described in Example 1A.

Materials and methods: A base guide vector with a polIII bicistronic transcript cassette coding EGFP followed by a lox2772-flanked IRES-PAC (puromycin resistance gene), followed, in its 3′UTR, by the empty guide expression cassette (U6-promoter driven, with the 10× feature capture sequence 1 in the hairpin position) was constructed using methods described in Example 1 (FIG. 1C). The U6-driven guide cassette allowed guide RNA expression using a polIII transcription system.

Various promoters were cloned upstream of the polII cassette after digestion with BstBI restriction enzyme (that recognizes TT\hat{ }CGAA sites) and AscI restriction enzyme (that recognizes GG\hat{ }CGCGCC sites). The constructs were generated using the same PCR-derived amplicons used for the Cas9 vector as described in Example 1. Guides were also cloned into this vector by adding two overlapping oligonucleotides.

Each guide sequence disclosed in Table 8 was introduced into separate guide constructs, then introduced into lentivirus as described above. Cas9 lines (comprising the construct shown in FIG. 1B) were transduced with the desired guide constructs (Table 9), followed by puromycin selection. Cells were maintained in both blasticidin and puromycin to maintain selection pressure.

TABLE 8
Sequences of spacers (DNA) and corresponding guide RNAs used in editing
experiments.

| | SEQ | | | | SEQ | |
|---|---|---|---|---|---|---|
| | ID | | | | ID | |
| Target | NO: | Protospacer | PAM | sgRNA | NO: | sgRNA sequence |
| Olfr | 12 | ATATTGGG | AGG | sgNEG1 | 13 | ATATTGGGAGGTATCTACACtgtttaag |
| 706 | | AGGTATCT | | | | agctaagctggaaacagcatagcaagtttaaataaggct |
| | | ACAC | | | | agtccgttatcaacttggccgctttaaggccggtcctagc |
| | | | | | | aaggccaagtggcaccgagtcggtgctttttt |
| Olfr | 14 | GCTACTCC | TGG | sgNEG2 | 15 | GCTACTCCTTGTCATCACAGtgtttaag |
| 706 | | TTGTCATC | | | | agctaagctggaaacagcatagcaagtttaaataaggct |
| | | ACAG | | | | agtccgttatcaacttggccgctttaaggccggtcctagc |
| | | | | | | aaggccaagtggcaccgagtcggtgctttttt |
| MIF | 16 | GCAAACCT | CGG | sgMIF1 | 17 | GCAAACCTGTGCGGGCTTGCtgtttaa |
| | | GTGCGGGC | | | | gagctaagctggaaacagcatagcaagtttaaataagg |
| | | TTGC | | | | ctagtccgttatcaacttggccgctttaaggccggtccta |
| | | | | | | gcaaggccaagtggcaccgagtcggtgctttttt |
| MIF | 18 | GGCCACCG | AGG | sgMIF2 | 19 | GGCCACCGGCAAGCCCGCACtgtttaa |
| | | GCAAGCCC | | | | gagctaagctggaaacagcatagcaagtttaaataagg |
| | | GCAC | | | | ctagtccgttatcaacttggccgctttaaggccggtccta |
| | | | | | | gcaaggccaagtggcaccgagtcggtgctttttt |
| MIF | 20 | CCAGTACA | TGG | sgMIF3 | 21 | CCAGTACATCGCAGTGCACGtgtttaa |
| | | TCGCAGTG | | | | gagctaagctggaaacagcatagcaagtttaaataagg |
| | | CACG | | | | ctagtccgttatcaacttggccgctttaaggccggtccta |
| | | | | | | gcaaggccaagtggcaccgagtcggtgctttttt |
| MIF | 22 | ACCGGTCC | AGG | sgMIF4 | 23 | ACCGGTCCGGGCTGATGTGCtgtttaa |
| | | GGGCTGAT | | | | gagctaagctggaaacagcatagcaagtttaaataagg |
| | | GTGC | | | | ctagtccgttatcaacttggccgctttaaggccggtccta |
| | | | | | | gcaaggccaagtggcaccgagtcggtgctttttt |
| MIF | 24 | ACAGCAGC | CGG | sgMIF5 | 25 | ACAGCAGCTTACTGTAGTTGtgtttaag |
| | | TTACTGTA | | | | agctaagctggaaacagcatagcaagtttaaataaggct |
| | | GTTG | | | | agtccgttatcaacttggccgctttaaggccggtcctagc |
| | | | | | | aaggccaagtggcaccgagtcggtgctttttt |
| MIF | 26 | CCCTCTGG | GGG | sgMIF6 | 27 | CCCTCTGGCACGGAGGCGCGtgtttaa |
| | | CACGGAGG | | | | gagctaagctggaaacagcatagcaagtttaaataagg |
| | | CGCG | | | | ctagtccgttatcaacttggccgctttaaggccggtccta |
| | | | | | | gcaaggccaagtggcaccgagtcggtgctttttt |
| MIF | 28 | CACAGCAT | TGG | sgMIF7 | 29 | CACAGCATCGGCAAGATCGGtgtttaa |
| | | CGGCAAGA | | | | gagctaagctggaaacagcatagcaagtttaaataagg |
| | | TCGG | | | | ctagtccgttatcaacttggccgctttaaggccggtccta |
| | | | | | | gcaaggccaagtggcaccgagtcggtgctttttt |
| MIF | 30 | CACGTGCA | GGG | sgMIF8 | 31 | CACGTGCACTGCGATGTACTtgtttaag |
| | | CTGCGATG | | | | agctaagctggaaacagcatagcaagtttaaataaggct |
| | | TACT | | | | agtccgttatcaacttggccgctttaaggccggtcctagc |
| | | | | | | aaggccaagtggcaccgagtcggtgctttttt |
| MIF | 32 | CCCCGCGC | GGG | sgMIF9 | 33 | CCCCGCGCCTCCGTGCCAGAtgtttaa |
| | | CTCCGTGC | | | | gagctaagctggaaacagcatagcaagtttaaataagg |
| | | CAGA | | | | ctagtccgttatcaacttggccgctttaaggccggtccta |
| | | | | | | gcaaggccaagtggcaccgagtcggtgctttttt |

TABLE 9
Elements of guide vector construct

| SEQ | | |
|---|---|---|
| ID NO: | Promoter | Sequence |
| 34 | U6 | gagggcctatttcccatgattccttcatatttgca |
| | promoter | tatacgatacaaggctgttagagagataattagaa |
| | | ttaatttgactgtaaacacaaagatattagtacaa |
| | | aatacgtgacgtagaaagtaataatttcttgggta |
| | | gtttgcagttttaaaattatgttttaaaatggact |
| | | atcatatgcttaccgtaacttgaaagtatttcgat |
| | | ttcttggctttatatatcttgtggaaaggac |
| 35 | Lox2772 | ATAACTTCGTATAGGATACTTTATACGAAGTTAT |
| 36 | Puromycin | atgaccgagtacaagcccacggtgcgcctcgccac |
| | | ccgcgacgacgtccccagggccgtacgcaccctcg |
| | | ccgccgcgttcgccgactaccccgccacgcgccac |
| | | accgtcgatccggaccgccacatcgagcgggtcac |
| | | cgagctgcaagaactcttcctcacgcgcgtcgggc |
| | | tcgacatcggcaaggtgtgggtcgcggacgacggc |
| | | gccgccgtggcggtctggaccacgccggagagcgt |
| | | cgaagcgggggcggtgttcgccgagatcggcccgc |
| | | gcatggccgagttgagcggttcccggctggccgcg |
| | | cagcaacagatggaaggcctcctggcgccgcaccg |
| | | gcccaaggagcccgcgtggttcctggccaccgtcg |
| | | gagtctcgcccgaccaccagggcaagggtctgggc |
| | | agcgccgtcgtgctccccggagtggaggcggccga |
| | | gcgcgccggggtgcccgccttcctggagacctccg |
| | | cgccccgcaacctccccttctacgagcggctcggc |
| | | ttcaccgtcaccgccgacgtcgaggtgcccgaagg |
| | | accgcgcacctggtgcatgacccgcaagcccggtg |
| | | cctga |
| 37 | WPRE | aatcaacctctggattacaaaatttgtgaaagatt |
| | | gactggtattcttaactatgttgctccttttacgc |
| | | tatgtggatacgctgctttaatgcctttgtatcat |
| | | gctattgcttcccgtatggctttcattttctcctc |
| | | cttgtataaatcctggttgctgtctctttatgagg |
| | | agttgtggcccgttgtcaggcaacgtggcgtggtg |
| | | tgcactgtgtttgctgacgcaacccccactggttg |
| | | gggcattgccaccacctgtcagctcctttccggga |
| | | ctttcgctttccccctccctattgccacggcggaa |
| | | ctcatcgccgcctgccttgcccgctgctggacagg |
| | | ggctcggctgttgggcactgacaattccgtggtgt |
| | | tgtcggggaaatcatcgtcctttccttggctgctc |
| | | gcctgtgttgccacctggattctgcgcgggacgtc |
| | | cttctgctacgtcccttcggccctcaatccagcgg |
| | | accttccttcccgcggcctgctgccggctctgcgg |
| | | cctcttccgcgtcttcgccttcgccctcagacgag |
| | | tcggatctccctttgggccgcctccccgc |
| 38 | EGFP | ATGGTGAGCAAGGGCGAGGAGCTGTTCACCGGGGT |
| | | GGTGCCCATCCTGGTCGAGCTGGACGGCGACGTAA |
| | | ACGGCCACAAGTTCAGCGTGTCCGGCGAGGGCGAG |
| | | GGCGATGCCACCTACGGCAAGCTGACCCTGAAGTT |
| | | CATCTGCACCACCGGCAAGCTGCCCGTGCCCTGGC |
| | | CCACCCTCGTGACCACCCTGACCTACGGCGTGCAG |
| | | TGCTTCAGCCGCTACCCCGACCACATGAAGCAGCA |
| | | CGACTTCTTCAAGTCCGCCATGCCCGAAGGCTACG |
| | | TCCAGGAGCGCACCATCTTCTTCAAGGACGACGGC |
| | | AACTACAAGACCCGCGCCGAGGTGAAGTTCGAGGG |
| | | CGACACCCTGGTGAACCGCATCGAGCTGAAGGGCA |
| | | TCGACTTCAAGGAGGACGGCAACATCCTGGGGCAC |
| | | AAGCTGGAGTACAACTACAACAGCCACAACGTCTA |
| | | TATCATGGCCGACAAGCAGAAGAACGGCATCAAGG |
| | | TGAACTTCAAGATCCGCCACAACATCGAGGACGGC |
| | | AGCGTGCAGCTCGCCGACCACTACCAGCAGAACAC |
| | | CCCCATCGGCGACGGCCCCGTGCTGCTGCCCGACA |
| | | ACCACTACCTGAGCACCCAGTCCGCCCTGAGCAAA |
| | | GACCCCAACGAGAAGCGCGATCACATGGTCCTGCT |
| | | GGAGTTCGTGACCGCCGCCGGGATCACTCTCGGCA |
| | | TGGACGAGCTGTACAAGTAA |
| 39 | IRES | tccctcccccccccctaacgttactggccgaagcc |
| | | gcttggaataaggccggtgtgcgtttgtctatatg |
| | | ttattttccaccatattgccgtcttttggcaatgt |
| | | gagggcccggaaacctggccctgtcttcttgacga |
| | | gcattcctaggggtctttcccctctcgccaaagga |
| | | atgcaaggtctgttgaatgtcgtgaaggaagcagt |
| | | tcctctggaagcttcttgaagacaaacaacgtctg |
| | | tagcgaccctttgcaggcagcggaaccccccacct |
| | | ggcgacaggtgcctctgcggccaaaagccacgtgt |
| | | ataagatacacctgcaaaggcggcacaaccccagt |
| | | gccacgttgtgagttggatagttgtggaaagagtc |
| | | aaatggctctcctcaagcgtattcaacaaggggct |
| | | gaaggatgcccagaaggtaccccattgtatgggat |
| | | ctgatctggggcctcggtgcacatgctttacgtgt |
| | | gtttagtcgaggttaaaaaaacgtctaggcccccc |
| | | gaaccacggggacgtggttttcctttgaaaaacac |
| | | gatgataat |
| 40 | 5′ LTR | gggtctctctggttagaccagatctgagcctggga |
| | | gctctctggctaactagggaacccactgcttaagc |
| | | ctcaataaagcttgccttgagtgcttcaagtagtg |
| | | tgtgcccgtctgttgtgtgactctggtaactagag |
| | | atccctcagacccttttagtcagtgtggaaaatct |
| | | ctagca |
| 41 | 3′ LTR + | tggaagggctaattcactcccaacgaagacaagat |
| | hU6 | ttcaccattatcgtttcagacccacctcccaaccc |
| | (hU6 is | cgaggggacccagagagggcctatttcccatgatt |
| | underlined) | ccttcatatttgcatatacgatacaaggctgttag |
| | | agagataattagaattaatttgactgtaaacacaa |
| | | agatattagtacaaaatacgtgacgtagaaagtaa |
| | | taatttcttgggtagtttgcagttttaaaattatg |
| | | ttttaaaatggactatcatatgcttaccgtaactt |
| | | gaaagtatttcgatttcttggctttatatatcttg |
| | | tggaaaggacgaaacaccggagacgcttttttcgt |
| | | ctctgtttaagagctaagctggaaacagcatagca |
| | | agtttaaataaggctagtccgttatcaacttggcc |
| | | gctttaaggccggtcctagcaaggccaagtggcac |
| | | cgagtcggtgcttttttaagcttggcgtaactaga |
| | | tcttgagacactgctttttgcttgtactgggtctc |
| | | tctggttagaccagatctgagcctgggagctctct |
| | | ggctaactagggaacccactgcttaagcctcaata |
| | | aagcttgccttgagtgcttcaagtagtgtgtgccc |
| | | gtctgttgtgtgactctggtaactagagatccctc |
| | | agacccttttagtcagtgtggaaaatctctagca |

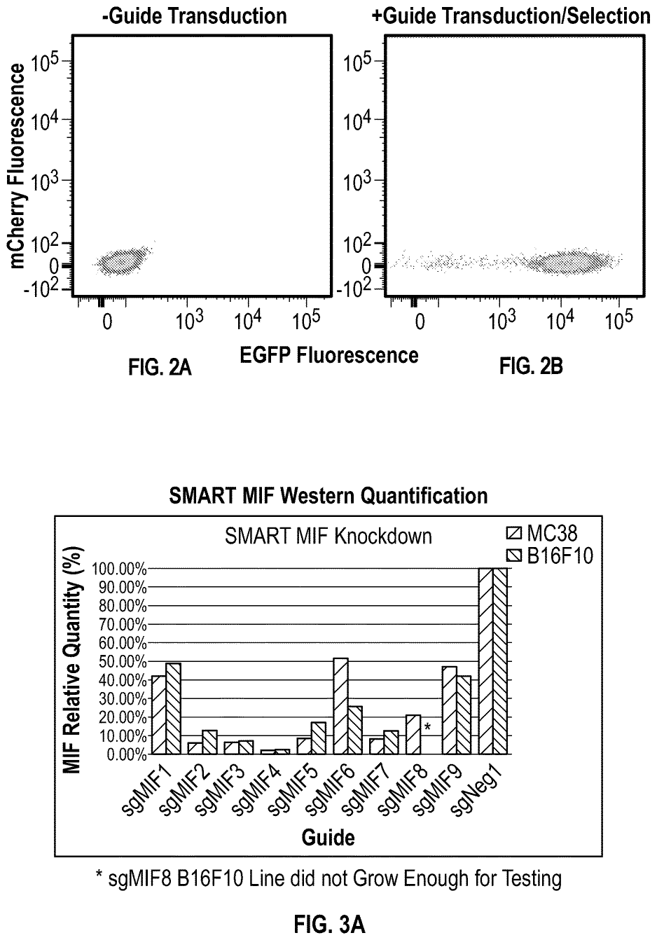

Results: Data were collected for the treated cells. EGFP fluorescence was observed in MC38 cells upon guide construct transduction and puromycin selection (FIG. 2). These data demonstrated that cells were efficiently transduced with guide constructs.

### Example 3: Analyzing Target Protein Knockdown Levels in Cells Treated with Cas9 Constructs

MC38 cells and B16F10 cells that were positive for both Cas9 and guide constructs were assayed for Cas9 mediated gene editing by identifying target protein levels.

Materials and methods: Cells were cultured under dual selection following guide construct transduction for 1-2 weeks, ~1 million cells were harvested and lysed in RIPA buffer for western analysis. B16F10 cells were cultured in Dulbecco's Modified Eagle Medium (DMEM), 10% fetal bovine serum (FBS), and penicillin-streptomycin.

MC38 cells were cultured in DMEM, 10% FBS, penicillin-streptomycin, 10 mM HEPES, minimal essential medium-non-Essential Amino Acids, 2 mM glutamine, and 1 mM sodium pyruvate.

Samples were analyzed for protein knockdown by western blot. The 3-actin level served as a loading control. The sgNEG1 level served as a negative control.

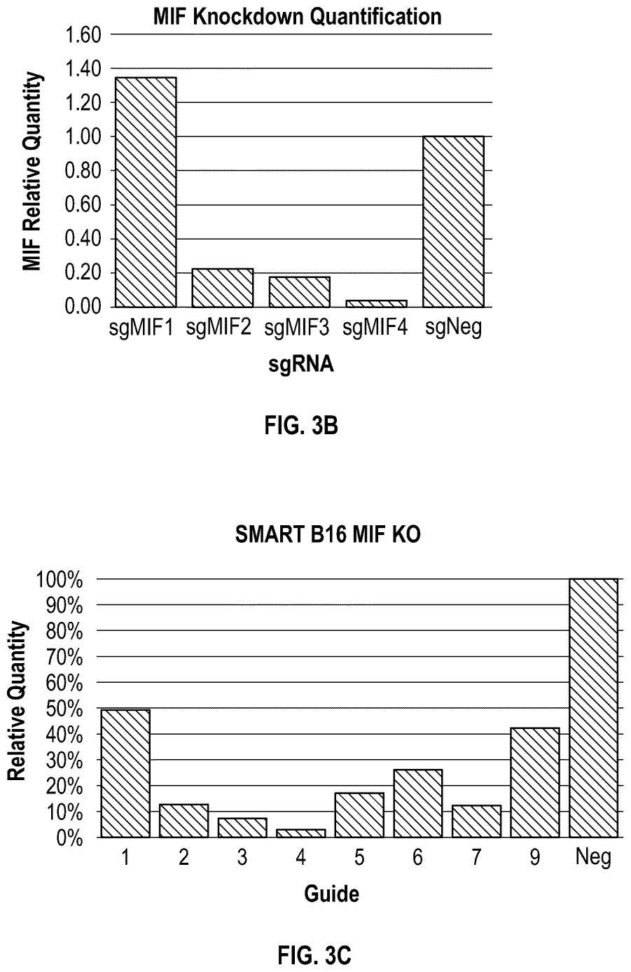

Results: The data show that the sgMIF2-5 and 7 constructs knocked down MIF levels in the B16F10 cells by at least five-fold compared to the negative control (sgNEG cell line). Robust MIF knockdown was observed for multiple sgMIF guides across both cell lines (FIGS. 3A-3C). The construct having SgMIF4 was the most efficient editor of both cell lines.

### Example 4: Determining Whether Selection Markers are Efficiently Removed by Transient Cre Expression

Cells carrying both Cas9 and guide constructs were further modified by removing the selection markers for both constructs.

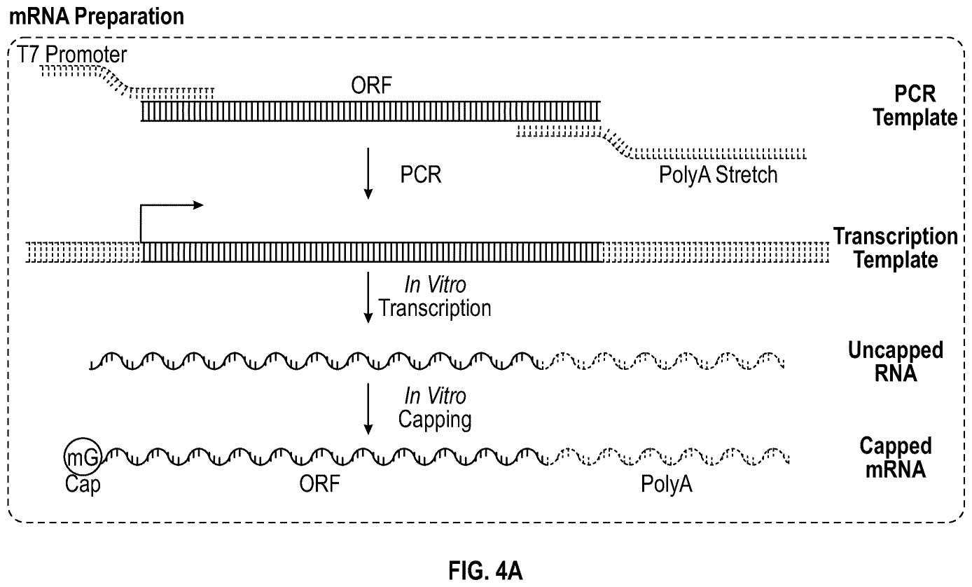

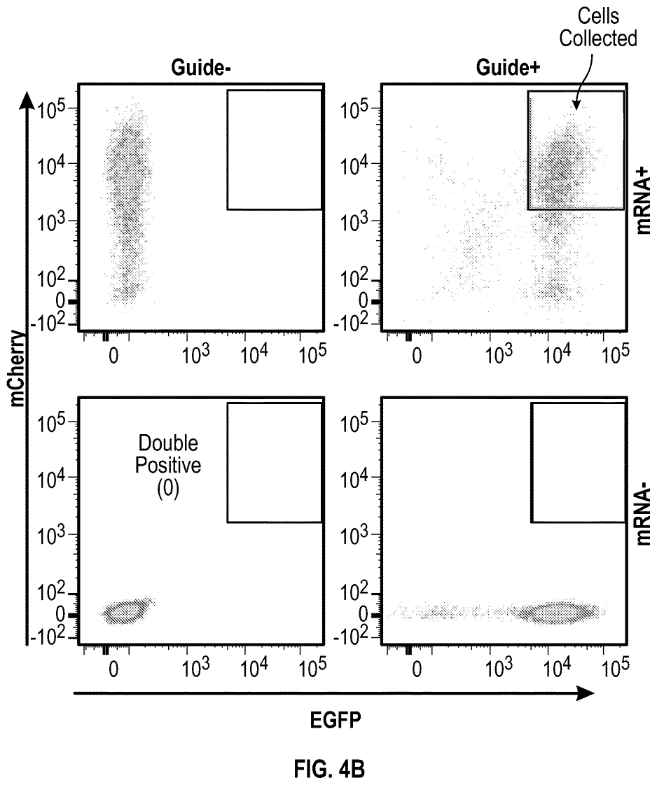

Materials and methods: Templates to produce Cre mRNA and mCherry mRNA were made by amplifying a Cre ORF and mCherry ORF by PCR with primers that added the T7 promoter upstream, and a 120 polyA stretch downstream. The sequences utilized are disclosed in Table 10. Amplicons were subsequently used as a template for in vitro transcription. Resulting transcripts were purified via lithium chloride precipitation, then capped using an in vitro capping reaction and column purified (FIG. 4A).

TABLE 10
Sequences used to make templates for in vitro transcription.

| SEQ | | |
|---|---|---|
| ID | | |
| NO: | Element | Sequence |
| 42 | Cre | ATGTCCAATTTACTGACCGTACACCAAAATTTGCCTGCATTACC |
| | mRNA | CGTCGATGCAACGAGTGATGAGGTTCGCAAGAACCTGATGGAT |
| | | ATGTTCAGGGATCGCCAGGCGTTTTCTGAGCATACCTGGAAAA |
| | | TGCTTCTGTCCGTTTGCCGGTCGTGGGCGGCATGGTGCAAGTTG |
| | | AATAACCGGAAATGGTTTCCCGCAGAACCTGAAGATGTTCGCG |
| | | ATTATCTTCTATATCTTCAGGCCCGCGGTCTGGCAGTAAAAACT |
| | | ATCCAGCAACATTTGGGCCAGCTAAACATGCTTCATCGTCGGT |
| | | CCGGGCTGCCACGACCAAGTGACAGCAATGCTGTTTCACTGGT |
| | | TATGCGGCGCATCCGAAAAGAAAACGTGGATGCCGGTGAACG |
| | | TGCAAAACAGGCTCTAGCGTTTGAACGCACTGATTTCGACCAG |
| | | GTTCGTTCACTCATGGAAAATAGCGACCGCTGCCAGGATATAC |
| | | GTAATCTGGCATTTCTGGGGATTGCTTACAACACCCTGTTACGT |
| | | ATAGCCGAAATTGCCAGGATCAGGGTTAAAGACATCTCACGTA |
| | | CTGACGGTGGGAGAATGTTAATCCATATTGGCAGAACGAAAAC |
| | | GCTGGTTAGCACCGCAGGTGTAGAGAAGGCACTTAGCCTGGGG |
| | | GTAACTAAACTGGTCGAGCGATGGATTTCCGTCTCTGGTGTAG |
| | | CTGATGATCCGAATAACTACCTGTTTTGCCGGGTCAGAAAAAA |
| | | TGGTGTTGCCGCGCCATCTGCCACCAGCCAGCTATCAACTCGC |
| | | GCCCTGGAAGGGATTTTTGAAGCAACTCACCGATTGATTTACG |
| | | GCGCTAAGGATGACTCTGGTCAGAGATACCTGGCCTGGTCTGG |
| | | ACACAGTGCCCGTGTCGGAGCCGCGCGAGATATGGCCCGCGCT |
| | | GGAGTTTCAATACCGGAGATCATGCAAGCTGGTGGCTGGACCA |
| | | ATGTAAATATTGTGATGAACTATATCCGTAACCTGGATAGTGA |
| | | AACAGGGGCAATGGTGCGCCTGCTGGAAGATGGCGACTAG |
| 43 | mCherry | ATGGTGAGCAAGGGCGAGGAGGATAACATGGCCATCATCAAG |
| | mRNA | GAGTTCATGCGCTTCAAGGTGCACATGGAGGGCTCCGTGAACG |
| | | GCCACGAGTTCGAGATCGAGGGCGAGGGCGAGGGCCGCCCCT |
| | | ACGAGGGCACCCAGACCGCCAAGCTGAAGGTGACCAAGGGTG |
| | | GCCCCCTGCCCTTCGCCTGGGACATCCTGTCCCCTCAGTTCATG |
| | | TACGGCTCCAAGGCCTACGTGAAGCACCCCGCCGACATCCCCG |
| | | ACTACTTGAAGCTGTCCTTCCCCGAGGGCTTCAAGTGGGAGCG |
| | | CGTGATGAACTTCGAGGACGGCGGCGTGGTGACCGTGACCCAG |
| | | GACTCCTCCCTGCAGGACGGCGAGTTCATCTACAAGGTGAAGC |
| | | TGCGCGGCACCAACTTCCCCTCCGACGGCCCCGTAATGCAGAA |
| | | GAAGACCATGGGCTGGGAGGCCTCCTCCGAGCGGATGTACCCC |
| | | GAGGACGGCGCCCTGAAGGGCGAGATCAAGCAGAGGCTGAAG |
| | | CTGAAGGACGGCGGCCACTACGACGCTGAGGTCAAGACCACC |
| | | TACAAGGCCAAGAAGCCCGTGCAGCTGCCCGGCGCCTACAAC |
| | | GTCAACATCAAGTTGGACATCACCTCCCACAACGAGGACTACA |
| | | CCATCGTGGAACAGTACGAACGCGCCGAGGGCCGCCACTCCAC |
| | | CGGCGGCATGGACGAGCTGTACAAGTCCGGACTCAGATCTCGA |
| | | TCTTTTTCCCTCTGCCAAAAATTATGGGGACATCATGAAGCCCC |
| | | TTGA |
| 44 | T7 | TAATACGACTCACTATAGGG |
| | promoter | |
| 45 | 120 polyA | AAAAAAAAAAAAAAAAAAAAAAAAAAAAAAAAAAAAAAAA |
| | | AAAAAAAAAAAAAAAAAAAAAAAAAAAAAAAAAAAAAAAA |
| | | AAAAAAAAAAAAAAAAAAAAAAAAAAAAAAAAAAAAAAAA |

Selection marker genes were removed by plating cells at a density of 125,000 cells per well of a 12-well plate without blasticidin or puromycin, then transfected with an mRNA mix of Cre mRNA and mCherry mRNA. Cre mRNA was used to remove selection marker genes, and mCherry mRNA was used as a marker for mRNA uptake.

To assay for successful transfection, EGFP+/mChey+ cells were isolated via FACS, and grown for several days to a week in media without puromycin or blasticidin.

To assay for successful selection marker removal, gDNA was extracted from pre- and post-selection marker removal cells and used as a template (250-500 ng/20 μl reaction, normalized across sample pairs) The ddCT method, with a Cas9 assay as an internal control, was used to determine the reduction in intact selection marker cassettes. The qPCR primers used in this Example are disclosed in Table 11.

TABLE 11
qPCR primers used to assay for selectable markers.

| SEQ ID NO: | Primer Name | Sequence |
|---|---|---|
| 46 | Cas9 Forward | CCCAAGAGGAACAGCGATAAG |
| 47 | Cas9 Reverse | CCACCACCAGCACAGAATAG |
| 48 | BSD cassette | ATTGCTGCCCTCTGGTTATG |
| | Forward | |
| 49 | BSD cassette | AGAGGGTTAGGGATAGGCTTAC |
| | Reverse | |
| 50 | PAC cassette | CATGGACGAGCTGTACAAGTAA |
| | Forward | |
| 51 | PAC cassette | ACACCGGCCTTATTCCAAG |
| | Reverse | |

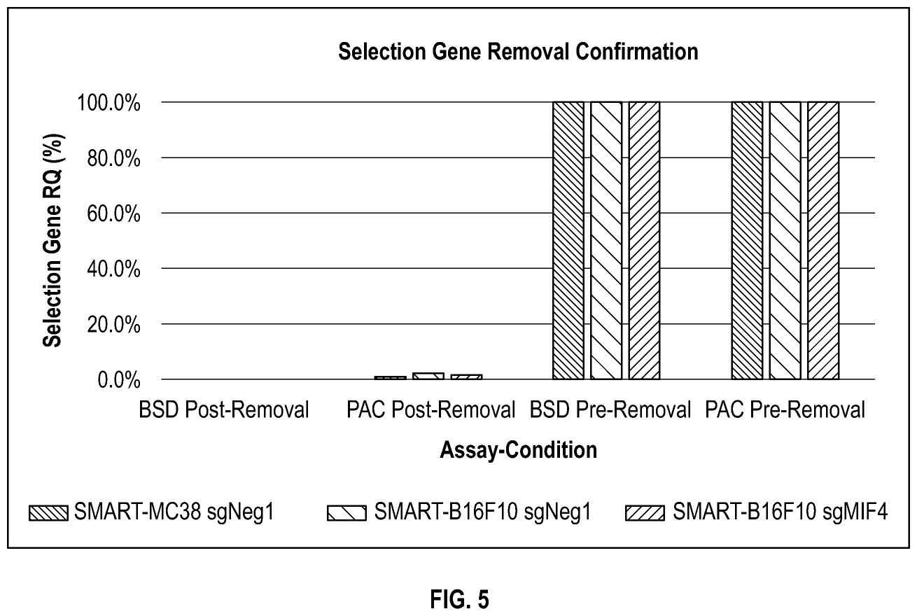

Results: Both MC38 cells and B16F10 cells contacted with the various versions of the system were efficiently transfected with mRNA and isolated 24 hours post mRNA transfection (FIG. 4B). Degradation of the transfected mRNAs was observed over the period of culturing post FACS isolation. Cells were also tested for the presence of loci corresponding to the PAC and BSD selectable markers. Data show both the excision and degradation of both selection gene cassettes from all the tested constructs (>98%) (FIG. 5).

### Example 5: Determination of Whether Engineered MC38 Cells are Tolerized by Subjects in a Cas9+/Egfp+ Mouse Model

Syngeneic MC38 cells carrying the Cas9 and guide vector system were injected into murine subjects to test whether removal of the antibiotic resistance genes reduced tumor rejection in Cas9 tolerized mice.

Materials and methods: Engineered MC38 cells were created using the protocol described in Examples 1 and 2, using a guide (sgNeg1) targeting Olfr706 (a non-expressed gene).

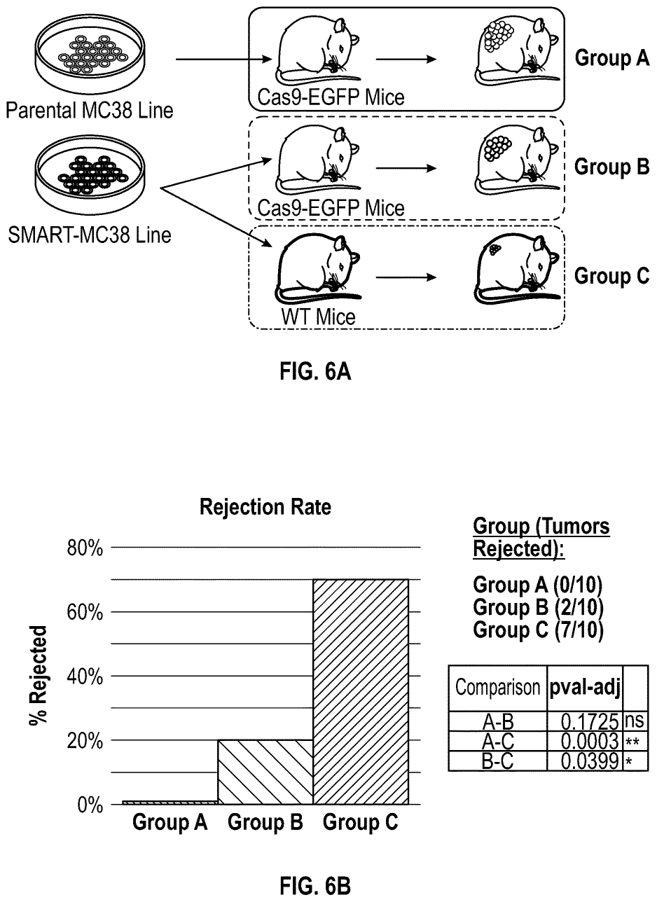

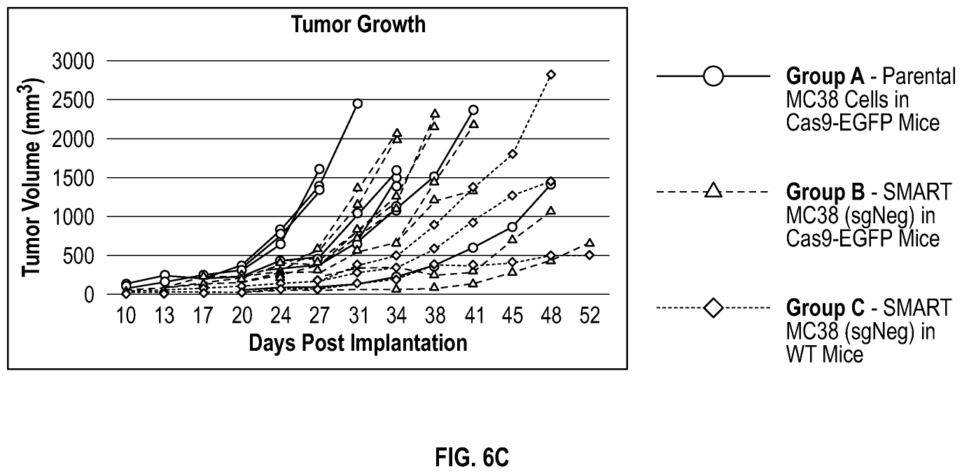

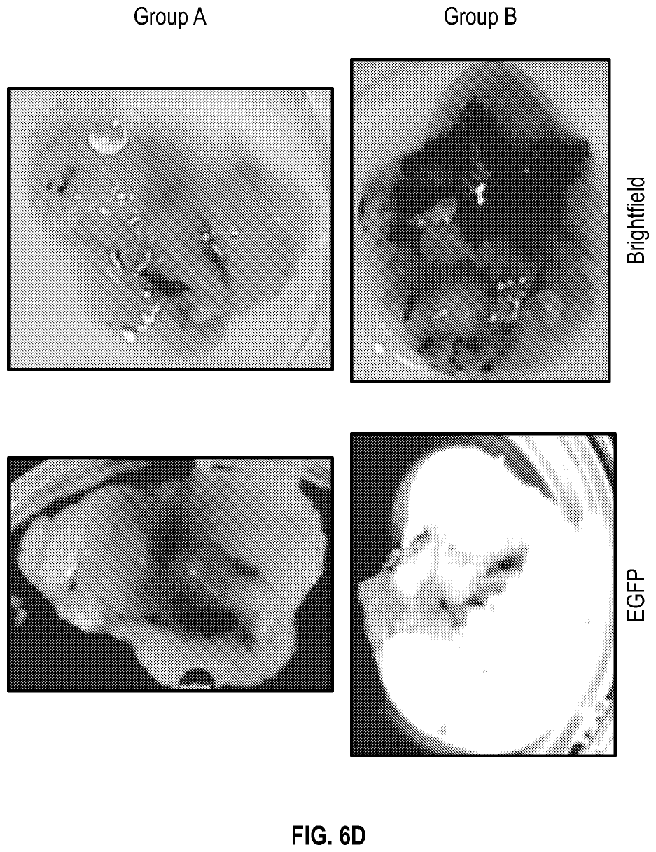

Cells were expanded, then an amount of cells (~1 million cells) were resuspended in 100 μl of cold phosphate buffered saline (PBS), and were implanted subcutaneously into the right flanks of either Cas9-EGFP tolerized mice (MT120-CAG-Cas9/Zp3-Cre) or wild-type (C57BL/6) mice (Group B and Group C, respectively, n=10 each) (FIG. 6A). Parental MC38 cells were implanted in Cas9-EGFP (MT120-CAG-Cas9/Zp3-Cre) mice (group A, n=10) (FIG. 6A).

For the following analysis, tumors were considered rejected if the tumors never grew to palpable size or shrank to a non-palpable size. Tumors were calipered and mice body weights were measured twice weekly, and tumor volumes were calculated using the formula: tumor volume=½(length×width²).

Results: Wild-type mice injected with engineered MC38 cells (Group C) rejected tumors the most, while Cas9+/EGFP+ mice were tolerant of parental MC38 cells (Group A) and engineered MC38 cells (Group B) as measured by a low tumor rejection rate (FIG. 6B and FIG. 6C). Data show that Groups A and B were more tolerant of their injected cells compared to Group C. Wild-type mice implanted with engineered MC38 cells had the highest tumor rejection rate out of the three experimental groups (FIG. 6B).

Tumors were resected from Group A murine subjects and Group B murine subjects. The tumors appeared approximately equivalent by brightfield microscopy (FIG. 6D). Only Group B tumors were observed to be EGFP+ indicating that these cells retained the guide vector (FIG. 6D).

### Example 6: Determination of Whether Engineered MC38 Cells Respond Equivalently to Anti-PD1 Immunotherapy

Syngeneic MC38 cells carrying the Cas9 and guide vector system were injected into murine subjects to test whether engineered cells, post-removal of the antibiotic resistance genes, responded equivalently to the parental MC38 cells to immunotherapy (αPD1 antibody treatment).

Materials and methods: Engineered MC38 cells were those created in Example 5. Parental MC38 cells were used as a control.

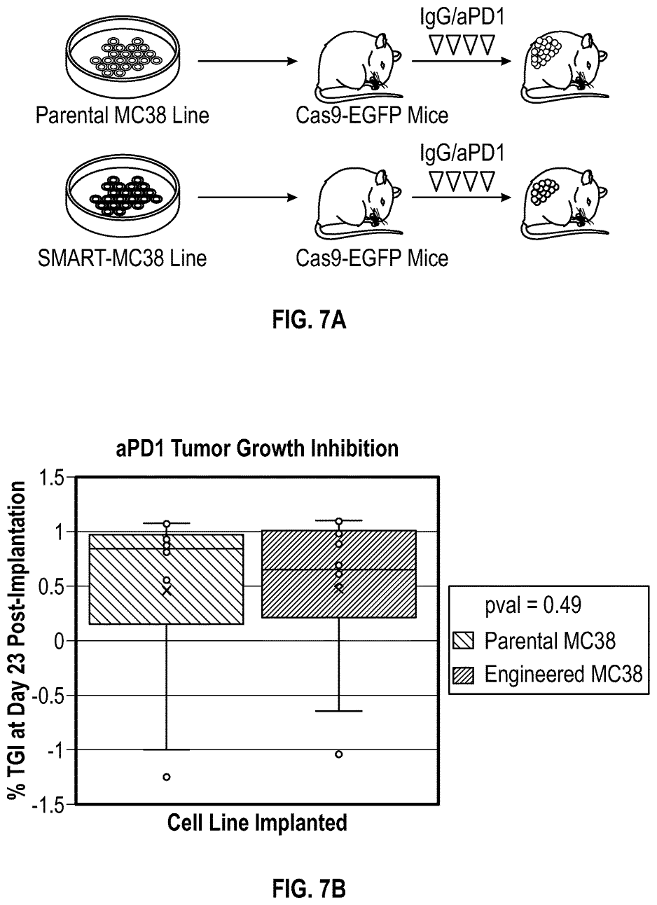

Cells were expanded, then an amount of cells (~1 million cells) were resuspended in 100 μl of cold phosphate buffered saline (PBS), and were implanted subcutaneously into the right flanks of Cas9-EGFP tolerized mice (MT120-CAG-Cas9/Zp3-Cre). Mice with subcutaneous tumors measuring ~80-120 mm³ at 9 days post-implantation were enrolled for treatment (20 mice per cell line). Mice were treated with either αPD1 antibody or a control IgG antibody at days 10, 13, 17, and 20 post implantation (intraperitoneal, 10 mg/kg of body weight) (FIG. 7A).

For the following analysis, percent tumor growth inhibition (% TGI) following treatment was calculated for each αPD1-treated tumor for day 23 post-implantation using the following formula:

$$\begin{matrix}\% TGI = \frac{\frac{\frac{{T}_{23}}{{T}_{9}}}{\frac{{C}_{23}}{{C}_{9}}}}{\frac{{C}_{23} - {C}_{9}}{{C}_{23}}} \\ {T}_{23} = Individual α PD 1 - treated tumor volume , day 23 \\ {T}_{9} = Individual α PD 1 - treated tumor volume , day 9 \\ {C}_{23} = Mean IgG - treated tumor volume , day 23 \\ {C}_{9} = Mean IgG - treated tumor volume , day 9.\end{matrix}$$

Results: Both parental and engineered MC38 tumors showed equivalent tumor growth inhibition in response to αPD1 treatment, 45.5% and 46.5%, respectively (p=0.49), indicating an equivalent response to this treatment (FIG. 7B).

### Example 7: Targeted In Vitro Crispr Screen in Syngeneic Model

This example analyzed whether a pool of the guide constructs could be efficiently introduced into syngeneic cells, and could be used to conduct an in vivo CRISPR screen. A pool of guide vectors was cloned and efficiently introduced into syngeneic cells (KP2) that were already carrying the Cas9 construct as described in Example 1A. KP2 is a pancreatic ductal adenocarcinoma (PDAC) cell line.

Materials and methods: A guide vector with the hEF1a promoter was linearized with BsmBI. For each guide (672 total, 6 guides per each gene, 112 genes targeted), two premixed oligos (structure described in Table 11A) were brought to 100 μM with 10 mM tris, pH=8, with 50 mM NaCl, heated to 95° C. for 45 minutes, and annealed by cooling slowly to room temperature for 45 minutes. Annealed oligos for each guide were pooled in an equimolar ratio and cloned into the linearized vector using Gibson assembly. Dilutions of the subsequent bacterial transformation were plated, and colonies counted, used to ensure a library complexity of over 1000 times the number of guides.

This pooled guide library was then introduced into lentivirus as described above. KP2 cells comprising Cas9 (such as the construct shown in FIG. 1B) were transduced with this guide construct pool followed by puromycin selection. Cells were maintained in both blasticidin and puromycin to maintain selection pressure.

Selection genes were removed by plating cells at a density of 7.5×10⁶ cells on a 150 mm plate without blasticidin or puromycin, then transfected with an mRNA mix of Cre mRNA and mCherry mRNA. Cre mRNA was used to remove selection genes, and mCherry mRNA was used as a marker for mRNA uptake.

To assay for successful transfection, 1×10⁶ EGFP+/mCherry+ cells were isolated via FACS, and grown for approximately one week without puromycin or blasticidin.

Cells were expanded, then an amount of cells (5×10⁵ cells) were resuspended in 100 μl of a 1:1 mixture of cold phosphate buffered saline (PBS) and Matrigel, and were implanted subcutaneously into the right flanks of Cas9-EGFP tolerized mice (MT120-CAG-Cas9/Zp3-Cre).

Cell pellets of cell mixture used for implantation were collected (n=3), and cells were maintained in culture for 12 doublings and pellets were again collected (n=3).

Tumors were collected 21 days (n=25) and 28 days (n=22) post-implantation. Tumors and cell pellets were lysed and gDNA was column purified from the lysate. The integrated guide protospacer regions were amplified from 12 μg of gDNA per tumor, using the indexed oligos indicated (Table 11B, with variable length stagger in the forward primer used to create sequence diversity for sequencing). The same oligos were used to amplify the guide protospacer regions from the plasmid DNA library. These PCR amplicons were sequenced using Illumina sequencing technology for guide quantification (Read 1-52 cycles; Index 1-8 cycles; Index 2-8 cycles). Each individual guide was counted and then transformed into a normalized ratio for the population as described below.

To evaluate the effect of guides on cell growth and/or fitness, the relative in vitro response was determined by comparing the abundance of guides in tumor cells grown in cell culture for 12 doublings to the plasmid library of guides.

To evaluate and identify guides that have a stronger effect in vivo than in vitro, the relative in vivo response was determined by comparing the abundance of guides in the mouse tumor to the abundance of guides in an in vitro cell culture (e.g, the in vitro arm). The in vitro arm was the same cell line used to grow the mouse tumor (KP2s) with the same guide library but grown in vitro for 12 doublings.

The in vivo response was plotted against the in vitro arm, and candidate genes were identified based on guides that had a stronger effect in vivo than in vitro.

TABLE 11A
Guide construction

| Source | Sequence | SEQ ID NO |
|---|---|---|
| Elements of Guide | ttaaac | N/A |
| Oligo 1 (5′-3′) | Protospacer reverse complement | |
| | cggtgtttcgtcctttcca | SEQ ID NO: 52 |
| Elements of Guide | Protospacer | N/A |
| oligo 2 (5′-3′) | gtttaagagctaagctggaaacagc | SEQ ID NO: 53 |
| Elements of Guide | AATGATACGGCGACCACCGA | SEQ ID NO: 54 |
| sequence | 8 bp index | |
| amplification primer | ACACTCTTTCCCTACACGACGCTCTTCCG | SEQ ID NO: 55 |
| forward | ATCT | |
| | Variable length stagger, 0-8 bp long | |
| | TTGTGGAAAGGACGAAAC*A*C*C*G | SEQ ID NO: 56 |
| | (* indicate phosphorothioate bonds) | |
| Elements of Guide | CAAGCAGAAGACGGCATACGAGAT | SEQ ID NO: 57 |
| sequence | 8 bp index | |
| amplification primer | GTGACTGGAGTTCAGACGTGTGCTCTTC | SEQ ID NO: 58 |
| reverse | CGATCTCAAGTTGATAACGGACT*A*G*C | |
| | *C | |
| | * indicate phosphorothioate bonds) | |

TABLE 11B
Construction of indexed oligos

| Source | Sequence | SEQ ID NO |
|---|---|---|
| Elements of Guide | AATGATACGGCGACCACCGA | SEQ ID NO: 59 |
| sequence | 8 bp index | |
| amplification | ACACTCTTTCCCTACACGACGCTCTTCCGAT | SEQ ID NO: 60 |
| primer | CT | |
| forward | Variable length stagger, 0-8 bp long | |
| | TTGTGGAAAGGACGAAAC*A*C*C*G | SEQ ID NO: 61 |
| | (* indicate phosphorothioate bonds) | |
| Elements of Guide | CAAGCAGAAGACGGCATACGAGAT | SEQ ID NO: 62 |
| sequence | 8 bp index | |
| amplification | GTGACTGGAGTTCAGACGTGTGCTCTTCCG | SEQ ID NO: 63 |
| primer | ATCTCAAGTTGATAACGGACT*A*G*C*C | |
| reverse | (* indicate phosphorothioate bonds) | |

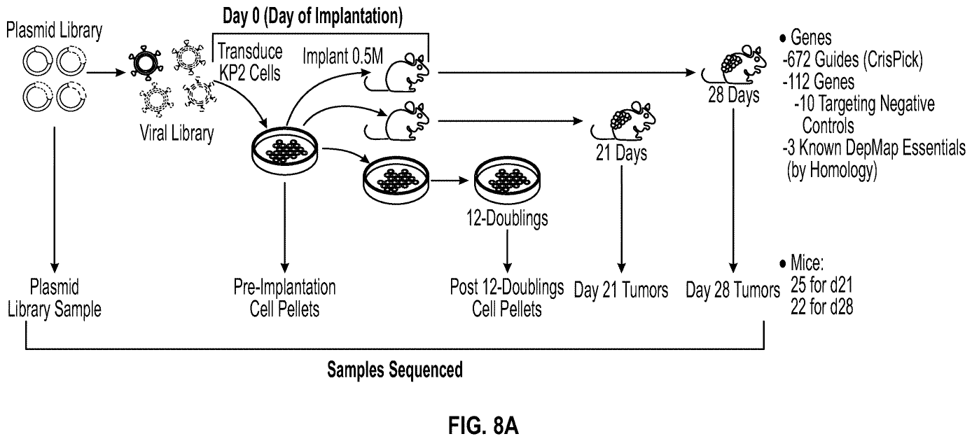

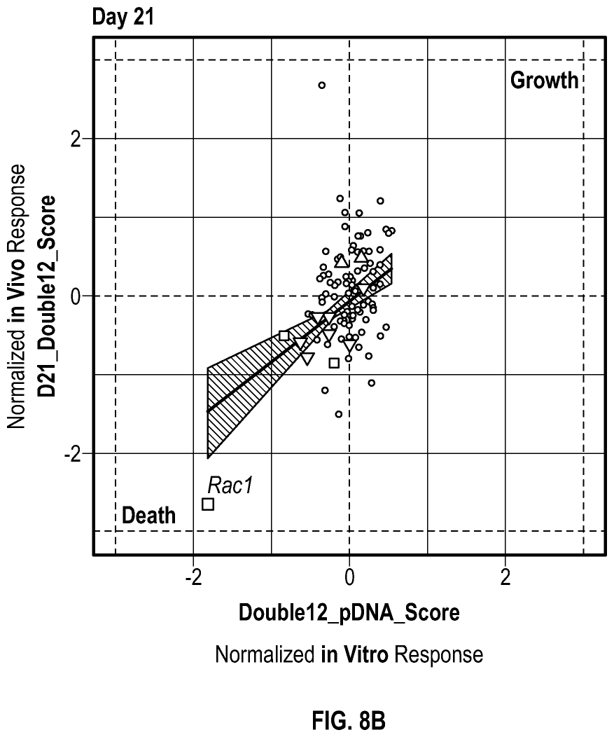

Results: Out of the 672 guides screened, 112 genes of interest were identified as effectors of tumor viability (FIG. 8A, FIG. 8B). 10 were known negative controls, and 3 were known Cancer Dependency Map (DepMap) essential genes. Of note, Rac1 is a strong essential gene and was isolated as a candidate from the screen (FIG. 8B). Generally, the 28 day in vivo phenotype matched the 21 day in vivo phenotype (data not shown). This demonstrates that the systems provided herein can be used to identify gene candidates associated with pancreatic ductal adenocarcinoma (PDAC).

Shown below are additional sequences included in the present application.

TABLE 12
Additional sequences

| # | SEQUENCE | ANNOTATION |
|---|---|---|
| 1 | GACATTGATTATTGACTAGTTATTAATAGTAATCAATTACGGGGTCAT | hCMV |
| | TAGTTCATAGCCCATATATGGAGTTCCGCGTTACATAACTTACGGTAA | promoter |
| | ATGGCCCGCCTGGCTGACCGCCCAACGACCCCCGCCCATTGACGTCA | |
| | ATAATGACGTATGTTCCCATAGTAACGCCAATAGGGACTTTCCATTGA | |
| | CGTCAATGGGTGGAGTATTTACGGTAAACTGCCCACTTGGCAGTACA | |
| | TCAAGTGTATCATATGCCAAGTCCGCCCCCTATTGACGTCAATGACGG | |
| | TAAATGGCCCGCCTGGCATTATGCCCAGTACATGACCTTACGGGACTT | |
| | TCCTACTTGGCAGTACATCTACGTATTAGTCATCGCTATTACCATGGT | |
| | GATGCGGTTTTGGCAGTACACCAATGGGCGTGGATAGCGGTTTGACT | |
| | CACGGGGATTTCCAAGTCTCCACCCCATTGACGTCAATGGGAGTTTGT | |
| | TTTGGCACCAAAATCAACGGGACTTTCCAAAATGTCGTAATAACCCC | |
| | GCCCCGTTGACGCAAATGGGCGGTAGGCGTGTACGGTGGGAGGTCTA | |
| | TATAAGCAGAGCTGGTTTAGTGAACCGTCAGATCCGCTAGCGCTACC | |
| | GGACTCAGATCTCGAGCTCAAGCTTCGAATTCTGCAGTCGACGGTAC | |
| | CGCGGGCCCGGGATCCACCGGTC | |
| 2 | GGGGTTGGGGTTGCGCCTTTTCCAAGGCAGCCCTGGGTTTGCGCAGG | hPGK |
| | GACGCGGCTGCTCTGGGCGTGGTTCCGGGAAACGCAGCGGCGCCGAC | promoter |
| | CCTGGGTCTCGCACATTCTTCACGTCCGTTCGCAGCGTCACCCGGATC | |
| | TTCGCCGCTACCCTTGTGGGCCCCCCGGCGACGCTTCCTGCTCCGCCC | |
| | CTAAGTCGGGAAGGTTCCTTGCGGTTCGCGGCGTGCCGGACGTGACA | |
| | AACGGAAGCCGCACGTCTCACTAGTACCCTCGCAGACGGACAGCGCC | |
| | AGGGAGCAATGGCAGCGCGCCGACCGCGATGGGCTGTGGCCAATAG | |
| | CGGCTGCTCAGCAGGGCGCGCCGAGAGCAGCGGCCGGGAAGGGGCG | |
| | GTGCGGGAGGCGGGGTGTGGGGCGGTAGTGTGGGCCCTGTTCCTGCC | |
| | CGCGCGGTGTTCCGCATTCTGCAAGCCTCCGGAGCGCACGTCGGCAG | |
| | TCGGCTCCCTCGTTGACCGAATCACCGACCTCTCTCCCCAG | |
| 3 | GGGTAGGGGAGGCGCTTTTCCCAAGGCAGTCTGGAGCATGCGCTTTA | mPGK |
| | GCAGCCCCGCTGGGCACTTGGCGCTACACAAGTGGCCTCTGGCCTCG | promoter |
| | CACACATTCCACATCCACCGGTAGGCGCCAACCGGCTCCGTTCTTTGG | |
| | TGGCCCCTTCGCGCCACCTTCTACTCCTCCCCTAGTCAGGAAGTTCCC | |
| | CCCCGCCCCGCAGCTCGCGTCGTGCAGGACGTGACAAATGGAAGTAG | |
| | CACGTCTCACTAGTCTCGTGCAGATGGACAGCACCGCTGAGCAATGG | |
| | AAGCGGGTAGGCCTTTGGTGCAGCGGCCAATAGCAGCTTTGCTCCTT | |
| | CGCTTTCTGGGCTCAGAGGCTGGGAAGGGGTGGGTCCGGGGGCGGGC | |
| | GCCCGAAGGTCCTCCGGAGGCCCGGCATTCTGCACGCTTCAAAAGCG | |
| | CACGTCTGCCGCGCTGTTCTCCTCTTCCTCATCTCCGGGCCTTTCG | |
| 4 | GGCTCCGGTGCCCGTCAGTGGGCAGAGCGCACATCGCCCACAGTCCC | hEF1a |
| | CGAGAAGTTGGGGGGAGGGGTCGGCAATTGAACCGGTGCCTAGAGA | promoter |
| | AGGTGGCGCGGGGTAAACTGGGAAAGTGATGTCGTGTACTGGCTCCG | |
| | CCTTTTTCCCGAGGGTGGGGGAGAACCGTATATAAGTGCAGTAGTCG | |
| | CCGTGAACGTTCTTTTTCGCAACGGGTTTGCCGCCAGAACACAGGTA | |
| | AGTGCCGTGTGTGGTTCCCGCGGGCCTGGCCTCTTTACGGGTTATGGC | |
| | CCTTGCGTGCCTTGAATTACTTCCACCTGGCTGCAGTACGTGATTCTT | |
| | GATCCCGAGCTTCGGGTTGGAAGTGGGTGGGAGAGTTCGAGGCCTTG | |
| | CGCTTAAGGAGCCCCTTCGCCTCGTGCTTGAGTTGAGGCCTGGCCTGG | |
| | GCGCTGGGGCCGCCGCGTGCGAATCTGGTGGCACCTTCGCGCCTGTC | |
| | TCGCTGCTTTCGATAAGTCTCTAGCCATTTAAAATTTTTGATGACCTG | |
| | CTGCGACGCTTTTTTTCTGGCAAGATAGTCTTGTAAATGCGGGCCAAG | |
| | ATCTGCACACTGGTATTTCGGTTTTTGGGGCCGCGGGCGGCGACGGG | |
| | GCCCGTGCGTCCCAGCGCACATGTTCGGCGAGGCGGGGCCTGCGAGC | |
| | GCGGCCACCGAGAATCGGACGGGGGTAGTCTCAAGCTGGCCGGCCTG | |
| | CTCTGGTGCCTGGCCTCGCGCCGCCGTGTATCGCCCCGCCCTGGGCGG | |
| | CAAGGCTGGCCCGGTCGGCACCAGTTGCGTGAGCGGAAAGATGGCCG | |
| | CTTCCCGGCCCTGCTGCAGGGAGCTCAAAATGGAGGACGCGGCGCTC | |
| | GGGAGAGCGGGCGGGTGAGTCACCCACACAAAGGAAAAGGGCCTTT | |
| | CCGTCCTCAGCCGTCGCTTCATGTGACTCCACGGAGTACCGGGCGCC | |
| | GTCCAGGCACCTCGATTAGTTCTCGAGCTTTTGGAGTACGTCGTCTTT | |
| | AGGTTGGGGGGAGGGGTTTTATGCGATGGAGTTTCCCCACACTGAGT | |
| | GGGTGGAGACTGAAGTTAGGCCAGCTTGGCACTTGATGTAATTCTCC | |
| | TTGGAATTTGCCCTTTTTGAGTTTGGATCTTGGTTCATTCTCAAGCCTC | |
| | AGACAGTGGTTCAAAGTTTTTTTCTTCCATTTCAGGTGTCGTGA | |
| 5 | AGAGTAATTCATACAAAAGGAGGGATCGCCTTCGCCTGGGGAAGTCC | mEFla |
| | CAGGGACCGTCGCTAAATTCTCATAACCCATAATCCCGGTACCCGCC | promoter |
| | CCACCACAGTGCGAGGAGCATGCGCTCAGGGCTGAGCGCGGGGAGA | |
| | GCAGAGCACACAAGCTCATAGACCCTGGTCGTGGGGGGAGGGGCGC | |
| | ACTGAGCGGGGGGGGGGGGGGTGATGGGGGGGAGGACCGGGGAGCT | |
| | GGCGCGGGGCAAACTGGGAAAGCGGTGTCGTGTGCTGGCTCCGCCCT | |
| | CTTCCCGAGGGTGGGGGAGAACGGTATATAAGTGCGGCAGTCGCCTT | |
| | GGACGTTCTTTTTCGCAACGGGTTTGCCGTCAGAACGCAGGTGAGGG | |
| | GCGGGTGTGGCTTCCGCGGGCCGCCGAGCTGGAGGTCCTGCTCCGAG | |
| | CGGGCCGGGCCCCGCTGTCGTCGGCGGGGATTAGCTGCGAGCATTCC | |
| | CGCTTCGAGTTGCGGGCGGCGCGGGAGGCAGAGTGCGAGGCCTAGC | |
| | GGCAACCCCGTAGCCTCGCCTCGTGTCCGGCTTGAGGCCTAGCGTGG | |
| | TGTCCGCGCCGCCGCCGCGTGCTACTCCGGCCGCACTCTGGTCTTTTT | |
| | TTTTTTGTTGTTGTTGTTGCCCTGCTGCCTTCGATTGCCGTTCAGCAAT | |
| | AGGGGCTAACAAAGGGAGGGTGCGGGGCTTGCTCGCCCGGAGCCCG | |
| | GAGAGGTCATGGTTGGGGAGGAATGGAGGGACAGGAGTGGCGGCTG | |
| | GGGCCCGCCCGCCTTCGGAGCACATGTCCGACGCCACCTGGATGGGG | |
| | CGAGGCCTGGGGTTTTTCCCGAAGCAACCAGGCTGGGGTTAGCGTGC | |
| | CGAGGCCATGTGGCCCCAGCACCCGGCACGATCTGGCTTGGCGGCGC | |
| | CGCGTTGCCCTGCCTCCCTAACTAGGGTGAGGCCATCCCGTCCGGCAC | |
| | CAGTTGCGTGCGTGGAAAGATGGCCGCTCCCGGGCCCTGTTGCAAGG | |
| | AGCTCAAAATGGAGGACGCGGCAGCCCGGTGGAGCGGGCGGGTGAG | |
| | TCACCCACACAAAGGAAGAGGGCCTGGTCCCTCACCGGCTGCTGCTT | |
| | CCTGTGACCCCGTGGTCCTATCGGCCGCAATAGTCACCTCGGGCTTTT | |
| | GAGCACGGCTAGTCGCGGCGGGGGGAGGGGATGTAATGGCGTTGGA | |
| | GTTTGTTCACATTTGGTGGGTGGAGACTAGTCAGGCCAGCCTGGCGCT | |
| | GGAAGTCATTTTTGGAATTTGTCCCCTTGAGTTTTGAGCGGAGCTAAT | |
| | TCTCGGGCTTCTTAGCGGTTCAAAGGTATCTTTTAAACCCTTTTTTAG | |
| | GTGTTGTGAAAACCACCGCTAATTCAAAGCAA | |
| 6 | ATGGTGAGCAAGGGCGACTATAAGGACCACGACGGAGACTACAAGG | Flag-Cas9 |
| | ATCATGATATTGATTACAAAGACGATGACGATAAGATGGCCCCAAAG | |
| | AAGAAGCGGAAGGTCGGTATCCACGGAGTCCCAGCAGCCGACAAGA | |
| | AGTACAGCATCGGCCTGGACATCGGCACCAACTCTGTGGGCTGGGCC | |
| | GTGATCACCGACGAGTACAAGGTGCCCAGCAAGAAATTCAAGGTGCT | |
| | GGGCAACACCGACCGGCACAGCATCAAGAAGAACCTGATCGGAGCC | |
| | CTGCTGTTCGACAGCGGCGAAACAGCCGAGGCCACCCGGCTGAAGAG | |
| | AACCGCCAGAAGAAGATACACCAGACGGAAGAACCGGATCTGCTAT | |
| | CTGCAAGAGATCTTCAGCAACGAGATGGCCAAGGTGGACGACAGCTT | |
| | CTTCCACAGACTGGAAGAGTCCTTCCTGGTGGAAGAGGATAAGAAGC | |
| | ACGAGCGGCACCCCATCTTCGGCAACATCGTGGACGAGGTGGCCTAC | |
| | CACGAGAAGTACCCCACCATCTACCACCTGAGAAAGAAACTGGTGGA | |
| | CAGCACCGACAAGGCCGACCTGCGGCTGATCTATCTGGCCCTGGCCC | |
| | ACATGATCAAGTTCCGGGGCCACTTCCTGATCGAGGGCGACCTGAAC | |
| | CCCGACAACAGCGACGTGGACAAGCTGTTCATCCAGCTGGTGCAGAC | |
| | CTACAACCAGCTGTTCGAGGAAAACCCCATCAACGCCAGCGGCGTGG | |
| | ACGCCAAGGCCATCCTGTCTGCCAGACTGAGCAAGAGCAGACGGCTG | |
| | GAAAATCTGATCGCCCAGCTGCCCGGCGAGAAGAAGAATGGCCTGTT | |
| | CGGAAACCTGATTGCCCTGAGCCTGGGCCTGACCCCCAACTTCAAGA | |
| | GCAACTTCGACCTGGCCGAGGATGCCAAACTGCAGCTGAGCAAGGAC | |
| | ACCTACGACGACGACCTGGACAACCTGCTGGCCCAGATCGGCGACCA | |
| | GTACGCCGACCTGTTTCTGGCCGCCAAGAACCTGTCCGACGCCATCCT | |
| | GCTGAGCGACATCCTGAGAGTGAACACCGAGATCACCAAGGCCCCCC | |
| | TGAGCGCCTCTATGATCAAGAGATACGACGAGCACCACCAGGACCTG | |
| | ACCCTGCTGAAAGCTCTCGTGCGGCAGCAGCTGCCTGAGAAGTACAA | |
| | AGAGATTTTCTTCGACCAGAGCAAGAACGGCTACGCCGGCTACATTG | |
| | ACGGCGGAGCCAGCCAGGAAGAGTTCTACAAGTTCATCAAGCCCATC | |
| | CTGGAAAAGATGGACGGCACCGAGGAACTGCTCGTGAAGCTGAACA | |
| | GAGAGGACCTGCTGCGGAAGCAGCGGACCTTCGACAACGGCAGCAT | |
| | CCCCCACCAGATCCACCTGGGAGAGCTGCACGCCATTCTGCGGCGGC | |
| | AGGAAGATTTTTACCCATTCCTGAAGGACAACCGGGAAAAGATCGAG | |
| | AAGATCCTGACCTTCCGCATCCCCTACTACGTGGGCCCTCTGGCCAGG | |
| | GGAAACAGCAGATTCGCCTGGATGACCAGAAAGAGCGAGGAAACCA | |
| | TCACCCCCTGGAACTTCGAGGAAGTGGTGGACAAGGGCGCTTCCGCC | |
| | CAGAGCTTCATCGAGCGGATGACCAACTTCGATAAGAACCTGCCCAA | |
| | CGAGAAGGTGCTGCCCAAGCACAGCCTGCTGTACGAGTACTTCACCG | |
| | TGTATAACGAGCTGACCAAAGTGAAATACGTGACCGAGGGAATGAG | |
| | AAAGCCCGCCTTCCTGAGCGGCGAGCAGAAAAAGGCCATCGTGGACC | |
| | TGCTGTTCAAGACCAACCGGAAAGTGACCGTGAAGCAGCTGAAAGA | |
| | GGACTACTTCAAGAAAATCGAGTGCTTCGACTCCGTGGAAATCTCCG | |
| | GCGTGGAAGATCGGTTCAACGCCTCCCTGGGCACATACCACGATCTG | |
| | CTGAAAATTATCAAGGACAAGGACTTCCTGGACAATGAGGAAAACG | |
| | AGGACATTCTGGAAGATATCGTGCTGACCCTGACACTGTTTGAGGAC | |
| | AGAGAGATGATCGAGGAACGGCTGAAAACCTATGCCCACCTGTTCGA | |
| | CGACAAAGTGATGAAGCAGCTGAAGCGGCGGAGATACACCGGCTGG | |
| | GGCAGGCTGAGCCGGAAGCTGATCAACGGCATCCGGGACAAGCAGT | |
| | CCGGCAAGACAATCCTGGATTTCCTGAAGTCCGACGGCTTCGCCAAC | |
| | AGAAACTTCATGCAGCTGATCCACGACGACAGCCTGACCTTTAAAGA | |
| | GGACATCCAGAAAGCCCAGGTGTCCGGCCAGGGCGATAGCCTGCACG | |
| | AGCACATTGCCAATCTGGCCGGCAGCCCCGCCATTAAGAAGGGCATC | |
| | CTGCAGACAGTGAAGGTGGTGGACGAGCTCGTGAAAGTGATGGGCC | |
| | GGCACAAGCCCGAGAACATCGTGATCGAAATGGCCAGAGAGAACCA | |
| | GACCACCCAGAAGGGACAGAAGAACAGCCGCGAGAGAATGAAGCGG | |
| | ATCGAAGAGGGCATCAAAGAGCTGGGCAGCCAGATCCTGAAAGAAC | |
| | ACCCCGTGGAAAACACCCAGCTGCAGAACGAGAAGCTGTACCTGTAC | |
| | TACCTGCAGAATGGGCGGGATATGTACGTGGACCAGGAACTGGACAT | |
| | CAACCGGCTGTCCGACTACGATGTGGACCATATCGTGCCTCAGAGCT | |
| | TTCTGAAGGACGACTCCATCGACAACAAGGTGCTGACCAGAAGCGAC | |
| | AAGAACCGGGGCAAGAGCGACAACGTGCCCTCCGAAGAGGTCGTGA | |
| | AGAAGATGAAGAACTACTGGCGGCAGCTGCTGAACGCCAAGCTGATT | |
| | ACCCAGAGAAAGTTCGACAATCTGACCAAGGCCGAGAGAGGCGGCC | |
| | TGAGCGAACTGGATAAGGCCGGCTTCATCAAGAGACAGCTGGTGGAA | |
| | ACCCGGCAGATCACAAAGCACGTGGCACAGATCCTGGACTCCCGGAT | |
| | GAACACTAAGTACGACGAGAATGACAAGCTGATCCGGGAAGTGAAA | |
| | GTGATCACCCTGAAGTCCAAGCTGGTGTCCGATTTCCGGAAGGATTTC | |
| | CAGTTTTACAAAGTGCGCGAGATCAACAACTACCACCACGCCCACGA | |
| | CGCCTACCTGAACGCCGTCGTGGGAACCGCCCTGATCAAAAAGTACC | |
| | CTAAGCTGGAAAGCGAGTTCGTGTACGGCGACTACAAGGTGTACGAC | |
| | GTGCGGAAGATGATCGCCAAGAGCGAGCAGGAAATCGGCAAGGCTA | |
| | CCGCCAAGTACTTCTTCTACAGCAACATCATGAACTTTTTCAAGACCG | |
| | AGATTACCCTGGCCAACGGCGAGATCCGGAAGCGGCCTCTGATCGAG | |
| | ACAAACGGCGAAACCGGGGAGATCGTGTGGGATAAGGGCCGGGATT | |
| | TTGCCACCGTGCGGAAAGTGCTGAGCATGCCCCAAGTGAATATCGTG | |
| | AAAAAGACCGAGGTGCAGACAGGCGGCTTCAGCAAAGAGTCTATCCT | |
| | GCCCAAGAGGAACAGCGATAAGCTGATCGCCAGAAAGAAGGACTGG | |
| | GACCCTAAGAAGTACGGCGGCTTCGACAGCCCCACCGTGGCCTATTC | |
| | TGTGCTGGTGGTGGCCAAAGTGGAAAAGGGCAAGTCCAAGAAACTG | |
| | AAGAGTGTGAAAGAGCTGCTGGGGATCACCATCATGGAAAGAAGCA | |
| | GCTTCGAGAAGAATCCCATCGACTTTCTGGAAGCCAAGGGCTACAAA | |
| | GAAGTGAAAAAGGACCTGATCATCAAGCTGCCTAAGTACTCCCTGTT | |
| | CGAGCTGGAAAACGGCCGGAAGAGAATGCTGGCCTCTGCCGGCGAA | |
| | CTGCAGAAGGGAAACGAACTGGCCCTGCCCTCCAAATATGTGAACTT | |
| | CCTGTACCTGGCCAGCCACTATGAGAAGCTGAAGGGCTCCCCCGAGG | |
| | ATAATGAGCAGAAACAGCTGTTTGTGGAACAGCACAAGCACTACCTG | |
| | GACGAGATCATCGAGCAGATCAGCGAGTTCTCCAAGAGAGTGATCCT | |
| | GGCCGACGCTAATCTGGACAAAGTGCTGTCCGCCTACAACAAGCACC | |
| | GGGATAAGCCCATCAGAGAGCAGGCCGAGAATATCATCCACCTGTTT | |
| | ACCCTGACCAATCTGGGAGCCCCTGCCGCCTTCAAGTACTTTGACACC | |
| | ACCATCGACCGGAAGAGGTACACCAGCACCAAAGAGGTGCTGGACG | |
| | CCACCCTGATCCACCAGAGCATCACCGGCCTGTACGAGACACGGATC | |
| | GACCTGTCTCAGCTGGGAGGCGACAAAAGGCCGGCGGCCACGAAAA | |
| | AGGCCGGCCAGGCAAAAAAGAAAAAGTAA | |
| 7 | ATAACTTCGTATAGCATACATTATACGAAGTTAT | LoxP |
| 8 | tccctcccccccccctaacgttactggccgaagccgcttggaataaggccggtgtgcgtttgtctatatgttat | IRES |
| | tttccaccatattgccgtcttttggcaatgtgagggcccggaaacctggccctgtcttcttgacgagcattcct | |
| | aggggtctttcccctctcgccaaaggaatgcaaggtctgttgaatgtcgtgaaggaagcagttcctctggaagc | |
| | ttcttgaagacaaacaacgtctgtagcgaccctttgcaggcagcggaaccccccacctggcgacaggtgcctct | |
| | gcggccaaaagccacgtgtataagatacacctgcaaaggcggcacaaccccagtgccacgttgtgagttggata | |
| | gttgtggaaagagtcaaatggctctcctcaagcgtattcaacaaggggctgaaggatgcccagaaggtacccca | |
| | ttgtatgggatctgatctggggcctcggtgcacatgctttacgtgtgtttagtcgaggttaaaaaaacgtctag | |
| | gccccccgaaccacggggacgtggttttcctttgaaaaacacgatgataat | |
| 9 | ATGGCCAAGCCTTTGTCTCAAGAAGAATCCACCCTCATTGAAAGAGC | BSD |
| | AACGGCTACAATCAACAGCATCCCCATCTCTGAAGACTACAGCGTCG | |
| | CCAGCGCAGCTCTCTCTAGCGACGGCCGCATCTTCACTGGTGTCAATG | |
| | TATATCATTTTACTGGGGGACCTTGTGCAGAACTCGTGGTGCTGGGCA | |
| | CTGCTGCTGCTGCGGCAGCTGGCAACCTGACTTGTATCGTCGCGATCG | |
| | GAAATGAGAACAGGGGCATCTTGAGCCCCTGCGGACGGTGCCGACA | |
| | GGTGCTTCTCGATCTGCATCCTGGGATCAAAGCCATAGTGAAGGACA | |
| | GTGATGGACAGCCGACGGCAGTTGGGATTCGTGAATTGCTGCCCTCT | |
| | GGTTATGTGTGGGAGGGCCTGCAGCTGCAGTAG | |
| 10 | TTCCGCGGCTCTTCC | Upstream |
| | | amplicon |
| | | primer |
| 11 | GCCCTTGCTCACCATGGTGGC | Downstream |
| | | amplicon |
| | | primer |
| 12 | ATATTGGGAGGTATCTACAC | NEG1 |
| | | protospacer |
| 13 | ATATTGGGAGGTATCTACACtgtttaagagctaagctggaaacagcatagcaagtttaaataagg | sgNEG1 |
| | ctagtccgttatcaacttggccgctttaaggccggtcctagcaaggccaagtggcaccgagtcggtg | |
| | ctttttt | |
| 14 | GCTACTCCTTGTCATCACAG | NEG2 |
| | | protospacer |
| 15 | GCTACTCCTTGTCATCACAGtgtttaagagctaagctggaaacagcatagcaagtttaaataagg | sgNEG2 |
| | ctagtccgttatcaacttggccgctttaaggccggtcctagcaaggccaagtggcaccgagtcggtg | |
| | ctttttt | |
| 16 | GCAAACCTGTGCGGGCTTGC | MIF1 |
| | | protospacer |
| 17 | GCAAACCTGTGCGGGCTTGCtgtttaagagctaagctggaaacagcatagcaagtttaaataagg | sgMIF1 |
| | ctagtccgttatcaacttggccgctttaaggccggtcctagcaaggccaagtggcaccgagtcggtg | |
| | ctttttt | |
| 18 | GGCCACCGGCAAGCCCGCAC | MIF2 |
| | | protospacer |
| 19 | GGCCACCGGCAAGCCCGCACtgtttaagagctaagctggaaacagcatagcaagtttaaataag | sgMIF2 |
| | gctagtccgttatcaacttggccgctttaaggccggtcctagcaaggccaagtggcaccgagtcgg | |
| | tgctttttt | |
| 20 | CCAGTACATCGCAGTGCACG | MIF3 |
| | | protospacer |
| 21 | CCAGTACATCGCAGTGCACGtgtttaagagctaagctggaaacagcatagcaagtttaaataagg | sgMIF3 |
| | ctagtccgttatcaacttggccgctttaaggccggtcctagcaaggccaagtggcaccgagtcggtg | |
| | ctttttt | |
| 22 | ACCGGTCCGGGCTGATGTGC | MIF4 |
| | | protospacer |
| 23 | ACCGGTCCGGGCTGATGTGCtgtttaagagctaagctggaaacagcatagcaagtttaaataagg | sgMIF4 |
| | ctagtccgttatcaacttggccgctttaaggccggtcctagcaaggccaagtggcaccgagtcggtg | |
| | ctttttt | |
| 24 | ACAGCAGCTTACTGTAGTTG | MIF5 |
| | | protospacer |
| 25 | ACAGCAGCTTACTGTAGTTGtgtttaagagctaagctggaaacagcatagcaagtttaaataagg | sgMIF5 |
| | ctagtccgttatcaacttggccgctttaaggccggtcctagcaaggccaagtggcaccgagtcggtg | |
| | ctttttt | |
| 26 | CCCTCTGGCACGGAGGCGCG | MIF6 |
| | | protospacer |
| 27 | CCCTCTGGCACGGAGGCGCGtgtttaagagctaagctggaaacagcatagcaagtttaaataagg | sgMIF6 |
| | ctagtccgttatcaacttggccgctttaaggccggtcctagcaaggccaagtggcaccgagtcggtg | |
| | ctttttt | |
| 28 | CACAGCATCGGCAAGATCGG | MIF7 |
| | | protospacer |
| 29 | CACAGCATCGGCAAGATCGGtgtttaagagctaagctggaaacagcatagcaagtttaaataag | sgMIF7 |
| | gctagtccgttatcaacttggccgctttaaggccggtcctagcaaggccaagtggcaccgagtcgg | |
| | tgctttttt | |
| 30 | CACGTGCACTGCGATGTACT | MIF8 |
| | | protospacer |
| 31 | CACGTGCACTGCGATGTACTtgtttaagagctaagctggaaacagcatagcaagtttaaataagg | sgMIF8 |
| | ctagtccgttatcaacttggccgctttaaggccggtcctagcaaggccaagtggcaccgagtcgg | |
| | tgctttttt | |
| 32 | CCCCGCGCCTCCGTGCCAGA | MIF9 |
| | | protospacer |
| 33 | CCCCGCGCCTCCGTGCCAGAtgtttaagagctaagctggaaacagcatagcaagtttaaataagg | sgMIF9 |
| | ctagtccgttatcaacttggccgctttaaggccggtcctagcaaggccaagtggcaccgagtcggtg | |
| | ctttttt | |
| 34 | gagggcctatttcccatgattccttcatatttgcatatacgatacaaggctgttagagagataat | U6 |
| | tagaattaatttgactgtaaacacaaagatattagtacaaaatacgtgacgtagaaagtaataattt | promoter |
| | cttgggtagtttgcagttttaaaattatgttttaaaatggactatcatatgcttaccgtaacttgaa | |
| | agtatttcgatttcttggctttatatatcttgtggaaaggac | |
| 35 | ATAACTTCGTATAGGATACTTTATACGAAGTTAT | Lox2772 |
| 36 | atgaccgagtacaagcccacggtgcgcctcgccacccgcgacgacgtccccagggccgtacgcaccctcgccg | Puromycin |
| | ccgcgttcgccgactaccccgccacgcgccacaccgtcgatccggaccgccacatcgagcgggtcaccgagct | |
| | gcaagaactcttcctcacgcgcgtcgggctcgacatcggcaaggtgtgggtcgcggacgacggcgccgccgtgg | |
| | cggtctggaccacgccggagagcgtcgaagcgggggcggtgttcgccgagatcggcccgcgcatggccgagtt | |
| | gagcggttcccggctggccgcgcagcaacagatggaaggcctcctggcgccgcaccggcccaaggagcccgc | |
| | gtggttcctggccaccgtcggagtctcgcccgaccaccagggcaagggtctgggcagcgccgtcgtgctccccg | |
| | gagtggaggcggccgagcgcgccggggtgcccgccttcctggagacctcegcgccccgcaacctccccttctac | |
| | gagcggctcggcttcaccgtcaccgccgacgtcgaggtgcccgaaggaccgcgcacctggtgcatgacccgca | |
| | agcccggtgcctga | |
| 37 | aatcaacctctggattacaaaatttgtgaaagattgactggtattcttaactatgttgctccttttacgctatgtg | WPRE |
| | gatacgctgctttaatgcctttgtatcatgctattgcttcccgtatggctttcattttctcctccttgtataaatc | |
| | ctggttgctgtctctttatgaggagttgtggcccgttgtcaggcaacgtggcgtggtgtgcactgtgtttgctgac | |
| | gcaacccccactggttggggcattgccaccacctgtcagctcctttccgggactttcgctttccccctccctattg | |
| | ccacggeggaactcategccgcctgccttgcccgctgctggacaggggctcggctgttgggcactgacaattccgt | |
| | ggtgttgtcggggaaatcatcgtcctttccttggctgctcgcctgtgttgccacctggattctgcgcgggacgtcc | |
| | ttctgctacgtcccttcggccctcaatccagcggaccttccttcccgcggcctgctgccggctctgcggcctcttc | |
| | cgcgtcttcgccttcgccctcagacgagtcggatctccctttgggccgcctccccgc | |
| 38 | ATGGTGAGCAAGGGCGAGGAGCTGTTCACCGGGGTGGTGCCCATCCT | EGFP |
| | GGTCGAGCTGGACGGCGACGTAAACGGCCACAAGTTCAGCGTGTCCG | |
| | GCGAGGGCGAGGGCGATGCCACCTACGGCAAGCTGACCCTGAAGTTC | |
| | ATCTGCACCACCGGCAAGCTGCCCGTGCCCTGGCCCACCCTCGTGAC | |
| | CACCCTGACCTACGGCGTGCAGTGCTTCAGCCGCTACCCCGACCACA | |
| | TGAAGCAGCACGACTTCTTCAAGTCCGCCATGCCCGAAGGCTACGTC | |
| | CAGGAGCGCACCATCTTCTTCAAGGACGACGGCAACTACAAGACCCG | |
| | CGCCGAGGTGAAGTTCGAGGGCGACACCCTGGTGAACCGCATCGAGC | |
| | TGAAGGGCATCGACTTCAAGGAGGACGGCAACATCCTGGGGCACAA | |
| | GCTGGAGTACAACTACAACAGCCACAACGTCTATATCATGGCCGACA | |
| | AGCAGAAGAACGGCATCAAGGTGAACTTCAAGATCCGCCACAACATC | |
| | GAGGACGGCAGCGTGCAGCTCGCCGACCACTACCAGCAGAACACCCC | |
| | CATCGGCGACGGCCCCGTGCTGCTGCCCGACAACCACTACCTGAGCA | |
| | CCCAGTCCGCCCTGAGCAAAGACCCCAACGAGAAGCGCGATCACATG | |
| | GTCCTGCTGGAGTTCGTGACCGCCGCCGGGATCACTCTCGGCATGGA | |
| | CGAGCTGTACAAGTAA | |
| 39 | tccctcccccccccctaacgttactggccgaagccgcttggaataaggccggtgtgcgtttgtctatatgttatttt | |
| | ccaccatattgccgtcttttggcaatgtgagggcccggaaacctggccctgtcttcttgacgagcattcctaggggt | |
| | ctttcccctctcgccaaaggaatgcaaggtctgttgaatgtcgtgaaggaagcagttcctctggaagcttcttgaag | |
| | acaaacaacgtctgtagcgaccctttgcaggcagcggaaccccccacctggcgacaggtgcctctgcggccaaaagc | |
| | cacgtgtataagatacacctgcaaaggcggcacaaccccagtgccacgttgtgagttggatagttgtggaaagagtca | |
| | aatggctctcctcaagcgtattcaacaaggggctgaaggatgcccagaaggtaccccattgtatgggatctgatctg | |
| | gggcctcggtgcacatgctttacgtgtgtttagtcgaggttaaaaaaacgtctaggccccccgaaccacggggacg | |
| | tggttttcctttgaaaaacacgatgataat | |
| 40 | gggtctctctggttagaccagatctgagcctgggagctctctggctaactagggaacccactgcttaagcctcaataa | 5′ LTR |
| | agcttgccttgagtgcttcaagtagtgtgtgcccgtctgttgtgtgactctggtaactagagatccctcagacccttt | |
| | tagtcagtgtggaaaatctctagca | |
| 41 | tggaagggctaattcactcccaacgaagacaagatttcaccattatcgtttcagacccacctcccaaccccgagggg | 3′ |
| | acccagagagggcctatttcccatgattccttcatatttgcatatacgatacaaggctgttagagagataattagaa | LTR + hU6 |
| | ttaatttgactgtaaacacaaagatattagtacaaaatacgtgacgtagaaagtaataatttcttgggtagtttgca | (hU6 is |
| | gttttaaaattatgttttaaaatggactatcatatgcttaccgtaacttgaaagtatttcgatttcttggctttata | underlined) |
| | tatcttgtggaaaggacgaaacaccggagacgcttttttcgtctctgtttaagagctaagctggaaacagcatagca | |
| | agtttaaataaggctagtccgttatcaacttggccgctttaaggccggtcctagcaaggccaagtggcaccgagtcg | |
| | gtgcttttttaagcttggcgtaactagatcttgagacactgctttttgcttgtactgggtctctctggttagaccag | |
| | atctgagcctgggagctctctggctaactagggaacccactgcttaagcctcaataaagcttgccttgagtgcttca | |
| | agtagtgtgtgcccgtctgttgtgtgactctggtaactagagatccctcagacccttttagtcagtgtggaaaatct | |
| | ctagca | |
| 42 | ATGTCCAATTTACTGACCGTACACCAAAATTTGCCTGCATTACCCGTC | Cre mRNA |
| | GATGCAACGAGTGATGAGGTTCGCAAGAACCTGATGGATATGTTCAG | |
| | GGATCGCCAGGCGTTTTCTGAGCATACCTGGAAAATGCTTCTGTCCGT | |
| | TTGCCGGTCGTGGGCGGCATGGTGCAAGTTGAATAACCGGAAATGGT | |
| | TTCCCGCAGAACCTGAAGATGTTCGCGATTATCTTCTATATCTTCAGG | |
| | CCCGCGGTCTGGCAGTAAAAACTATCCAGCAACATTTGGGCCAGCTA | |
| | AACATGCTTCATCGTCGGTCCGGGCTGCCACGACCAAGTGACAGCAA | |
| | TGCTGTTTCACTGGTTATGCGGCGCATCCGAAAAGAAAACGTGGATG | |
| | CCGGTGAACGTGCAAAACAGGCTCTAGCGTTTGAACGCACTGATTTC | |
| | GACCAGGTTCGTTCACTCATGGAAAATAGCGACCGCTGCCAGGATAT | |
| | ACGTAATCTGGCATTTCTGGGGATTGCTTACAACACCCTGTTACGTAT | |
| | AGCCGAAATTGCCAGGATCAGGGTTAAAGACATCTCACGTACTGACG | |
| | GTGGGAGAATGTTAATCCATATTGGCAGAACGAAAACGCTGGTTAGC | |
| | ACCGCAGGTGTAGAGAAGGCACTTAGCCTGGGGGTAACTAAACTGGT | |
| | CGAGCGATGGATTTCCGTCTCTGGTGTAGCTGATGATCCGAATAACTA | |
| | CCTGTTTTGCCGGGTCAGAAAAAATGGTGTTGCCGCGCCATCTGCCAC | |
| | CAGCCAGCTATCAACTCGCGCCCTGGAAGGGATTTTTGAAGCAACTC | |
| | ACCGATTGATTTACGGCGCTAAGGATGACTCTGGTCAGAGATACCTG | |
| | GCCTGGTCTGGACACAGTGCCCGTGTCGGAGCCGCGCGAGATATGGC | |
| | CCGCGCTGGAGTTTCAATACCGGAGATCATGCAAGCTGGTGGCTGGA | |
| | CCAATGTAAATATTGTGATGAACTATATCCGTAACCTGGATAGTGAA | |
| | ACAGGGGCAATGGTGCGCCTGCTGGAAGATGGCGACTAG | |
| 43 | ATGGTGAGCAAGGGCGAGGAGGATAACATGGCCATCATCAAGGAGT | mCherry |
| | TCATGCGCTTCAAGGTGCACATGGAGGGCTCCGTGAACGGCCACGAG | mRNA |
| | TTCGAGATCGAGGGCGAGGGCGAGGGCCGCCCCTACGAGGGCACCC | |
| | AGACCGCCAAGCTGAAGGTGACCAAGGGTGGCCCCCTGCCCTTCGCC | |
| | TGGGACATCCTGTCCCCTCAGTTCATGTACGGCTCCAAGGCCTACGTG | |
| | AAGCACCCCGCCGACATCCCCGACTACTTGAAGCTGTCCTTCCCCGA | |
| | GGGCTTCAAGTGGGAGCGCGTGATGAACTTCGAGGACGGCGGCGTGG | |
| | TGACCGTGACCCAGGACTCCTCCCTGCAGGACGGCGAGTTCATCTAC | |
| | AAGGTGAAGCTGCGCGGCACCAACTTCCCCTCCGACGGCCCCGTAAT | |
| | GCAGAAGAAGACCATGGGCTGGGAGGCCTCCTCCGAGCGGATGTACC | |
| | CCGAGGACGGCGCCCTGAAGGGCGAGATCAAGCAGAGGCTGAAGCT | |
| | GAAGGACGGCGGCCACTACGACGCTGAGGTCAAGACCACCTACAAG | |
| | GCCAAGAAGCCCGTGCAGCTGCCCGGCGCCTACAACGTCAACATCAA | |
| | GTTGGACATCACCTCCCACAACGAGGACTACACCATCGTGGAACAGT | |
| | ACGAACGCGCCGAGGGCCGCCACTCCACCGGCGGCATGGACGAGCT | |
| | GTACAAGTCCGGACTCAGATCTCGATCTTTTTCCCTCTGCCAAAAATT | |
| | ATGGGGACATCATGAAGCCCCTTGA | |
| 44 | TAATACGACTCACTATAGGG | T7 |
| | | promoter |
| 45 | AAAAAAAAAAAAAAAAAAAAAAAAAAAAAAAAAAAAAAAA | 120 polyA |
| | AAAAAAAAAAAAAAAAAAAAAAAAAAAAAAAAAAAAAAAA | |
| | AAAAAAAAAAAAAAAAAAAAAAAAAAAAAAAAAAAAAAAA | |
| 46 | CCCAAGAGGAACAGCGATAAG | Cas9 |
| | | Forward |
| 47 | CCACCACCAGCACAGAATAG | Cas9 |
| | | Reverse |
| 48 | ATTGCTGCCCTCTGGTTATG | BSD |
| | | cassette |
| | | Forward |
| 49 | AGAGGGTTAGGGATAGGCTTAC | BSD |
| | | cassette |
| | | Reverse |
| 50 | CATGGACGAGCTGTACAAGTAA | PAC |
| | | cassette |
| | | Forward |
| 51 | ACACCGGCCTTATTCCAAG | PAC |
| | | cassette |
| | | Reverse |
| 52 | cggtgtttcgtcctttcca | Component |
| | | of Guide |
| | | oligo 1 |
| 53 | gtttaagagctaagctggaaacagc | Component |
| | | of guide |
| | | oligo 2 |
| 54 | AATGATACGGCGACCACCGA | Elements |
| | | of Guide |
| | | sequence |
| | | amplification |
| | | primer |
| | | forward |
| 55 | ACACTCTTTCCCTACACGACGCTCTTCCGATCT | Elements |
| | | of Guide |
| | | sequence |
| | | amplification |
| | | primer |
| | | forward |
| 56 | TTGTGGAAAGGACGAAACACCG | Elements |
| | | of Guide |
| | | sequence |
| | | amplification |
| | | primer |
| | | forward |
| 57 | CAAGCAGAAGACGGCATACGAGAT | Elements |
| | | of Guide |
| | | sequence |
| | | amplification |
| | | primer |
| | | reverse |
| 58 | GTGACTGGAGTTCAGACGTGTGCTCTTCCGATCTCAAGTTGATAACGG | Elements |
| | ACTAGCC | of Guide |
| | | sequence |
| | | amplification |
| | | primer |
| | | reverse |
| 59 | AATGATACGGCGACCACCGA | Elements |
| | | of Guide |
| | | sequence |
| | | amplification |
| | | primer |
| | | forward |
| 60 | ACACTCTTTCCCTACACGACGCTCTTCCGATCT | Elements |
| | | of Guide |
| | | sequence |
| | | amplification |
| | | primer |
| | | forward |
| 61 | TTGTGGAAAGGACGAAACACCG | Elements |
| | | of Guide |
| | | sequence |
| | | amplification |
| | | primer |
| | | forward |
| 62 | CAAGCAGAAGACGGCATACGAGAT | Elements |
| | | of Guide |
| | | sequence |
| | | amplification |
| | | primer |
| | | reverse |
| 63 | GTGACTGGAGTTCAGACGTGTGCTCTTCCGATCTCAAGTTGATAACGG | Elements |
| | ACTAGCC | of Guide |
| | | sequence |
| | | amplification |
| | | primer |
| | | reverse |

1. A vector comprising i) a nucleic acid encoding a Cas enzyme and/or or a nucleic acid encoding a guide RNA and ii) a nucleic acid encoding a selectable marker, wherein the nucleic acid encoding the selectable marker is flanked by recognition sites for a site-specific recombinase and wherein the nucleic acid encoding the Cas enzyme or the nucleic acid encoding the guide RNA is located outside of the recognition sites for the site-specific recombinase.

2. The vector of claim 1, wherein the vector comprises a nucleic acid encoding a Cas enzyme, wherein the Cas enzyme is a type I, type II, type Ill, type IV, or type V Cas enzyme.

3. The vector of claim 2, wherein the Cas enzyme is a Cas9, a Cas10, or a Cas12.

4. The vector of claim 2, further comprising a promoter operably linked to the nucleic acid encoding the Cas enzyme and the selectable marker.

5. The vector of claim 1, wherein the vector comprises a nucleic acid encoding a guide RNA.

6. The vector of claim 5, wherein the nucleic acid encoding the guide RNA is operably linked to a PolIII promoter.

7. The vector of claim 1, wherein the selectable marker comprises an antibiotic resistance gene.

8. The vector of claim 7, wherein the antibiotic resistance gene is BSD or PAC.

9. The vector of claim 4, wherein the promoter is an inducible promoter.

10. (canceled)

11. The vector of claim 4, wherein the promoter is selected form the group consisting of hCMV, hPGK, mPGK, hEF1a, and mEF1a.

12. (canceled)

13. The vector of claim 1, further comprising nucleic acid encoding a detection marker.

14. The vector of claim 13, wherein the nucleic acid encoding the detection marker is flanked by recognition sites for the site-specific recombinase.

15. The vector of claim 13, wherein the nucleic acid encoding the detection marker and the nucleic acid encoding the selection marker are flanked by the same recognition sites for the site-specific recombinase or
wherein the nucleic acid encoding the detection marker and the selection marker are flanked by different recognition sites for a site-specific recombinase.

16. The vector of claim 13, wherein the detection marker is a fluorescent protein, optionally wherein the detection marker is GFP, YFP, RFP, EGFP, or a luciferase.

17. The vector of claim 1, comprising a nucleic acid encoding an internal ribosome entry site between the nucleic acid encoding the Cas enzyme and the selectable marker.

18. The vector of claim 1, wherein the site specific recombinase is selected from the group consisting of Cre, Dre, and Flp.

19. The vector of claim 1, wherein the vector is a viral vector.

20. The vector of claim 19, wherein the vector is an adenoviral or a lentiviral vector.

21. A system comprising the vector of claim 1.

22. The system of claim 21, comprising a first vector comprising the nucleic acid encoding the Cas enzyme and a first selectable marker and a second vector comprising the nucleic acid encoding the guide RNA and a second selectable marker.

23. The system of claim 22, wherein the first selectable marker and the second selectable marker are different.

24. An engineered cell comprising the vector of claim 21.

25. The engineered cell of claim 24, wherein the cell is a human cell or a murine cell.

26. The engineered cell of claim 24, wherein the cell is a cell from a primary tumor or a tumor cell line.

27. (canceled)

28. A method of generating cells comprising a nucleic acid encoding a Cas enzyme comprising
removing nucleic acid encoding a selectable marker from a vector within a plurality of cells, wherein the vector comprises a nucleic acid encoding a Cas enzyme, a nucleic acid encoding a guide RNA, and the nucleic acid encoding the selectable marker, wherein the resulting cells comprise engineered cells comprising the nucleic acid encoding the Cas enzyme.

29. The method of claim 28, wherein prior to removing, the method comprises selecting the plurality of cells comprising the nucleic acid encoding the Cas enzyme, the nucleic acid encoding the guide RNA, and the nucleic acid encoding the selectable marker, wherein the nucleic acid encoding the selectable marker is removed by providing a site-specific recombinase to the plurality of cells.

30. The method of claim 29, wherein the site-specific recombinase is selected from the group consisting of Cre, Dre, and Flp.

31. The method of claim 29, wherein providing the site-specific recombinase to the cell comprises providing protein or mRNA encoding the site-specific recombinase to the cell plurality of cells.

32. The method of claim 31, further comprising performing in vitro transcription to produce the mRNA.

33. The method of claim 28, further comprising detecting removal of the nucleic acid encoding the selectable marker.

34. The method of claim 30, wherein detecting removal of the selectable marker comprises assaying for nucleic acid encoding the selectable marker.

35. The method of claim 29, wherein prior to selecting or removing, the method comprises transducing or transfecting the cell plurality of cells with one or more vectors comprising the nucleic acid encoding the Cas enzyme, the nucleic acid encoding the guide RNA, and the nucleic acid encoding the selectable marker.

36. The method of claim 28, comprising providing two or more vectors to the cell, wherein at least one vector comprises the nucleic acid encoding the Cas enzyme, at least one vector comprises the nucleic acid encoding the guide RNA, and at least one vector comprises the nucleic acid encoding the selectable marker.

37. The method of claim 36, wherein the two or more vectors comprises a selectable marker.

38. The method of claim 36, wherein each of the selectable markers is flanked by recognition sites for the site-specific recombinase.

39. The method of claim 37, wherein the selectable marker comprises two or more distinct selection markers.

40. The method of claim 36, wherein the nucleic acid encoding the Cas enzyme and the nucleic acid encoding the guide RNA are provided on two different vectors.

41. The method of claim 36, comprising providing first and second vectors, wherein the first vector comprises the nucleic acid encoding the Cas enzyme and nucleic acid encoding a first selectable marker flanked by recognition sites for a site specific-recombinase and
wherein the second vector comprises the nucleic acid encoding the guide RNA and nucleic acid encoding a second selectable marker flanked by recognition sites for a site-specific recombinase.

42. The method of claim 41, wherein the nucleic acid encoding the guide RNA and the nucleic acid encoding the Cas enzyme are each operably linked to a promoter.

43. The method of claim 28, wherein the selectable markers are removed following selecting the cells for the selectable marker.

44. The method of claim 28, further comprising selecting the cells for the selectable marker.

45. The method of claim 44, wherein selecting the cells for the selectable marker comprises culturing the cell with an antibiotic.

46. The method of claim 44, further comprising culturing the cell following selection with the selectable marker.

47. The method of claim 28, wherein the Cas enzyme is Cas9.

48. The method of claim 28, further comprising assaying the Cas enzyme activity in the engineered cells.

49. The method of claim 28, wherein the nucleic acid encoding the guide RNA is not removed.

50. The method of claim 28, wherein the one or more vectors comprises nucleic acid encoding a detection marker.

51. The method of claim 50, wherein the nucleic acid encoding the detection marker is flanked by recognition sites for the site specific recombinase.

52. The method of claim 50, wherein the detection marker is a fluorescent protein.

53. The method of claim 50, wherein the detection marker is GFP, YFP, RFP, EGFP, or a luciferase.

54. The method of claim 50, further comprising detecting the detection marker.

55. The method of claim 28, wherein the engineered cell is a primary tumor cell or a tumor cell line.

56. The method of claim 28, wherein the engineered cell is a murine cell or a human cell.

57. The method of claim 28, wherein the nucleic acid encoding the guide RNA is operably linked to a PolIII promoter.

58. The method of claim 57, wherein the promoter is an inducible promoter.

59. The method of claim 28, wherein the nucleic acid encoding the Cas enzyme is operably linked to an inducible promoter.

60. The method of any one of claims 57-59, wherein the promoter is a human promoter or a murine promoter.

61. (canceled)

62. (canceled)

63. (canceled)

64. The method of claim 28, wherein the engineered cells are engineered inducible cells that can be induced to express the Cas enzyme after introduction into an organism.

65. A method of introducing engineered cells comprising a Cas enzyme and a guide RNA into an organism that has an immune response, comprising
generating the engineered cells by
providing one or more vectors comprising the Cas enzyme, the guide RNA, and a selectable marker to a plurality of cells,
culturing the cells such that the plurality of cells express the selectable marker,
isolating from the cultured cells, selected cells the express the selectable marker,
removing the selectable marker from the isolated, selected cells to produce engineered cells,
providing the engineered cells to the organism, wherein the organism is tolerized to the Cas enzyme.

66. The method of claim 65, further comprising tolerizing the organism to the Cas enzyme.

67. The method of claim 66, wherein tolerizing the organism to the Cas enzyme comprises expressing the Cas enzyme in the organism prior to providing the engineered cells to the organism.

68. The method of claim 65, wherein the engineered cell is implanted or injected into the organism.

69. The method of claim 65, further comprising culturing the engineered cells prior to providing the engineered cells to the organism.

70. The method of claim 65, wherein expression of Cas enzyme and/or the guide RNA are operably linked to an inducible promoter.

71. The method of claim 70, further comprising activating the inducible promoter in the organism.

72. The method of claim 65, wherein expression of the nucleic acid encoding the Cas enzyme and the nucleic acid encoding the guide RNA results in editing in the genome of the engineered cell.

73. The method of claim 65, wherein the organism is a non-human mammal.

74. The method of claim 73, wherein the organism is a rat or a mouse.

75. The method of claim 65, wherein the organism is the same species as the engineered cell.

76. The method of claim 65, wherein the organism is a different species from the cell.

77. The method of claim 65, wherein the Cas enzyme is a Cas9.

78. The method of claim 65, wherein the engineered cells are mammalian cells.

79. The method of any claim 78, wherein the mammalian cells are murine cells or human cells.

80. The method of claim 65, wherein the engineered cells do not express a selectable marker.

81. (canceled)

82. The method of claim 65, wherein the rate of rejection of the engineered cells by the organism is reduced compared to rate of rejection of the engineered cells introduced into an organism which is not tolerized.

83. The method of claim 82, wherein the rejection rate of the engineered cells by the organism is reduced by at least 20% compared to rate of rejection of the engineered cells introduced into an organism which is not tolerized.

84. The method of claim 82, wherein the rejection rate of the engineered cells by the organism is reduced by at least 50% to rate of rejection of the engineered cells introduced into an organism which is not tolerized.

85. The method of claim 65, wherein the engineered cells are tumor cells and the rate of tumor growth in the organism is increased compared to the rate of tumor growth of the engineered cells into an organism which is not tolerized.

86. A kit comprising the vector of claim 1 and instructions for use.

87. (canceled)

88. (canceled)


SEQUENCE LISTING (63 sequences)

SEQ ID NO: 1 - DNA, 688 bp, synthetic construct
  source 1..688: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtc

SEQ ID NO: 2 - DNA, 511 bp, synthetic construct
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 3 - DNA, 471 bp, synthetic construct
  source 1..471: mol_type = other DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggtgcagcggccaatagcagctttgctccttcgctttctgggctcagaggctgggaaggggtgggtccgggggcgggcgcccgaaggtcctccggaggcccggcattctgcacgcttcaaaagcgcacgtctgccgcgctgttctcctcttcctcatctccgggcctttcg

SEQ ID NO: 4 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 5 - DNA, 1348 bp, synthetic construct
  source 1..1348: mol_type = other DNA
agagtaattcatacaaaaggagggatcgccttcgcctggggaagtcccagggaccgtcgctaaattctcataacccataatcccggtacccgccccaccacagtgcgaggagcatgcgctcagggctgagcgcggggagagcagagcacacaagctcatagaccctggtcgtggggggaggggcgcactgagcggggggggggggggtgatgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtctttttttttttgttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaa

SEQ ID NO: 6 - DNA, 4284 bp, synthetic construct
  source 1..4284: mol_type = other DNA
atggtgagcaagggcgactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaa

SEQ ID NO: 7 - DNA, 34 bp, synthetic construct
  source 1..34: mol_type = other DNA
ataacttcgtatagcatacattatacgaagttat

SEQ ID NO: 8 - DNA, 569 bp, synthetic construct
  source 1..569: mol_type = other DNA
tccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaaacgtctaggccccccgaaccacggggacgtggttttcctttgaaaaacacgatgataat

SEQ ID NO: 9 - DNA, 411 bp, synthetic construct
  source 1..411: mol_type = other DNA
atggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggcctgcagctgcagtag

SEQ ID NO: 10 - DNA, 15 bp, synthetic construct
  source 1..15: mol_type = other DNA
ttccgcggctcttcc

SEQ ID NO: 11 - DNA, 21 bp, synthetic construct
  source 1..21: mol_type = other DNA
gcccttgctcaccatggtggc

SEQ ID NO: 12 - DNA, 20 bp, synthetic construct
  source 1..20: mol_type = other DNA
atattgggaggtatctacac

SEQ ID NO: 13 - DNA, 139 bp, synthetic construct
  source 1..139: mol_type = other DNA
atattgggaggtatctacactgtttaagagctaagctggaaacagcatagcaagtttaaataaggctagtccgttatcaacttggccgctttaaggccggtcctagcaaggccaagtggcaccgagtcggtgctttttt

SEQ ID NO: 14 - DNA, 20 bp, synthetic construct
  source 1..20: mol_type = other DNA
gctactccttgtcatcacag

SEQ ID NO: 15 - DNA, 139 bp, synthetic construct
  source 1..139: mol_type = other DNA
gctactccttgtcatcacagtgtttaagagctaagctggaaacagcatagcaagtttaaataaggctagtccgttatcaacttggccgctttaaggccggtcctagcaaggccaagtggcaccgagtcggtgctttttt

SEQ ID NO: 16 - DNA, 20 bp, synthetic construct
  source 1..20: mol_type = other DNA
gcaaacctgtgcgggcttgc

SEQ ID NO: 17 - DNA, 139 bp, synthetic construct
  source 1..139: mol_type = other DNA
gcaaacctgtgcgggcttgctgtttaagagctaagctggaaacagcatagcaagtttaaataaggctagtccgttatcaacttggccgctttaaggccggtcctagcaaggccaagtggcaccgagtcggtgctttttt

SEQ ID NO: 18 - DNA, 20 bp, synthetic construct
  source 1..20: mol_type = other DNA
ggccaccggcaagcccgcac

SEQ ID NO: 19 - DNA, 139 bp, synthetic construct
  source 1..139: mol_type = other DNA
ggccaccggcaagcccgcactgtttaagagctaagctggaaacagcatagcaagtttaaataaggctagtccgttatcaacttggccgctttaaggccggtcctagcaaggccaagtggcaccgagtcggtgctttttt

SEQ ID NO: 20 - DNA, 20 bp, synthetic construct
  source 1..20: mol_type = other DNA
ccagtacatcgcagtgcacg

SEQ ID NO: 21 - DNA, 139 bp, synthetic construct
  source 1..139: mol_type = other DNA
ccagtacatcgcagtgcacgtgtttaagagctaagctggaaacagcatagcaagtttaaataaggctagtccgttatcaacttggccgctttaaggccggtcctagcaaggccaagtggcaccgagtcggtgctttttt

SEQ ID NO: 22 - DNA, 20 bp, synthetic construct
  source 1..20: mol_type = other DNA
accggtccgggctgatgtgc

SEQ ID NO: 23 - DNA, 139 bp, synthetic construct
  source 1..139: mol_type = other DNA
accggtccgggctgatgtgctgtttaagagctaagctggaaacagcatagcaagtttaaataaggctagtccgttatcaacttggccgctttaaggccggtcctagcaaggccaagtggcaccgagtcggtgctttttt

SEQ ID NO: 24 - DNA, 20 bp, synthetic construct
  source 1..20: mol_type = other DNA
acagcagcttactgtagttg

SEQ ID NO: 25 - DNA, 139 bp, synthetic construct
  source 1..139: mol_type = other DNA
acagcagcttactgtagttgtgtttaagagctaagctggaaacagcatagcaagtttaaataaggctagtccgttatcaacttggccgctttaaggccggtcctagcaaggccaagtggcaccgagtcggtgctttttt

SEQ ID NO: 26 - DNA, 20 bp, synthetic construct
  source 1..20: mol_type = other DNA
ccctctggcacggaggcgcg

SEQ ID NO: 27 - DNA, 139 bp, synthetic construct
  source 1..139: mol_type = other DNA
ccctctggcacggaggcgcgtgtttaagagctaagctggaaacagcatagcaagtttaaataaggctagtccgttatcaacttggccgctttaaggccggtcctagcaaggccaagtggcaccgagtcggtgctttttt

SEQ ID NO: 28 - DNA, 20 bp, synthetic construct
  source 1..20: mol_type = other DNA
cacagcatcggcaagatcgg

SEQ ID NO: 29 - DNA, 139 bp, synthetic construct
  source 1..139: mol_type = other DNA
cacagcatcggcaagatcggtgtttaagagctaagctggaaacagcatagcaagtttaaataaggctagtccgttatcaacttggccgctttaaggccggtcctagcaaggccaagtggcaccgagtcggtgctttttt

SEQ ID NO: 30 - DNA, 20 bp, synthetic construct
  source 1..20: mol_type = other DNA
cacgtgcactgcgatgtact

SEQ ID NO: 31 - DNA, 139 bp, synthetic construct
  source 1..139: mol_type = other DNA
cacgtgcactgcgatgtacttgtttaagagctaagctggaaacagcatagcaagtttaaataaggctagtccgttatcaacttggccgctttaaggccggtcctagcaaggccaagtggcaccgagtcggtgctttttt

SEQ ID NO: 32 - DNA, 20 bp, synthetic construct
  source 1..20: mol_type = other DNA
ccccgcgcctccgtgccaga

SEQ ID NO: 33 - DNA, 139 bp, synthetic construct
  source 1..139: mol_type = other DNA
ccccgcgcctccgtgccagatgtttaagagctaagctggaaacagcatagcaagtttaaataaggctagtccgttatcaacttggccgctttaaggccggtcctagcaaggccaagtggcaccgagtcggtgctttttt

SEQ ID NO: 34 - DNA, 241 bp, synthetic construct
  source 1..241: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 35 - DNA, 34 bp, synthetic construct
  source 1..34: mol_type = other DNA
ataacttcgtataggatactttatacgaagttat

SEQ ID NO: 36 - DNA, 600 bp, synthetic construct
  source 1..600: mol_type = other DNA
atgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgccgtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 37 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 38 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 39 - DNA, 569 bp, synthetic construct; identical to SEQ ID NO: 8
  source 1..569: mol_type = other DNA

SEQ ID NO: 40 - DNA, 181 bp, synthetic construct
  source 1..181: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 41 - DNA, 699 bp, synthetic construct
  source 1..699: mol_type = other DNA
tggaagggctaattcactcccaacgaagacaagatttcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagacgcttttttcgtctctgtttaagagctaagctggaaacagcatagcaagtttaaataaggctagtccgttatcaacttggccgctttaaggccggtcctagcaaggccaagtggcaccgagtcggtgcttttttaagcttggcgtaactagatcttgagacactgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 42 - DNA, 1032 bp, synthetic construct
  source 1..1032: mol_type = other DNA
atgtccaatttactgaccgtacaccaaaatttgcctgcattacccgtcgatgcaacgagtgatgaggttcgcaagaacctgatggatatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcccgcggtctggcagtaaaaactatccagcaacatttgggccagctaaacatgcttcatcgtcggtccgggctgccacgaccaagtgacagcaatgctgtttcactggttatgcggcgcatccgaaaagaaaacgtggatgccggtgaacgtgcaaaacaggctctagcgtttgaacgcactgatttcgaccaggttcgttcactcatggaaaatagcgaccgctgccaggatatacgtaatctggcatttctggggattgcttacaacaccctgttacgtatagccgaaattgccaggatcagggttaaagacatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcaccgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtgatgaactatatccgtaacctggatagtgaaacaggggcaatggtgcgcctgctggaagatggcgactag

SEQ ID NO: 43 - DNA, 774 bp, synthetic construct
  source 1..774: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtccggactcagatctcgatctttttccctctgccaaaaattatggggacatcatgaagccccttga

SEQ ID NO: 44 - DNA, 20 bp, synthetic construct
  source 1..20: mol_type = other DNA
taatacgactcactataggg

SEQ ID NO: 45 - DNA, 120 bp, synthetic construct
  source 1..120: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 46 - DNA, 21 bp, synthetic construct
  source 1..21: mol_type = other DNA
cccaagaggaacagcgataag

SEQ ID NO: 47 - DNA, 20 bp, synthetic construct
  source 1..20: mol_type = other DNA
ccaccaccagcacagaatag

SEQ ID NO: 48 - DNA, 20 bp, synthetic construct
  source 1..20: mol_type = other DNA
attgctgccctctggttatg

SEQ ID NO: 49 - DNA, 22 bp, synthetic construct
  source 1..22: mol_type = other DNA
agagggttagggataggcttac

SEQ ID NO: 50 - DNA, 22 bp, synthetic construct
  source 1..22: mol_type = other DNA
catggacgagctgtacaagtaa

SEQ ID NO: 51 - DNA, 19 bp, synthetic construct
  source 1..19: mol_type = other DNA
acaccggccttattccaag

SEQ ID NO: 52 - DNA, 19 bp, synthetic construct
  source 1..19: mol_type = other DNA
cggtgtttcgtcctttcca

SEQ ID NO: 53 - DNA, 25 bp, synthetic construct
  source 1..25: mol_type = other DNA
gtttaagagctaagctggaaacagc

SEQ ID NO: 54 - DNA, 20 bp, synthetic construct
  source 1..20: mol_type = other DNA
aatgatacggcgaccaccga

SEQ ID NO: 55 - DNA, 33 bp, synthetic construct
  source 1..33: mol_type = other DNA
acactctttccctacacgacgctcttccgatct

SEQ ID NO: 56 - DNA, 22 bp, synthetic construct
  source 1..22: mol_type = other DNA
  modified_base 18..22: mod_base = OTHER; note = phosphorothioate bonds
ttgtggaaaggacgaaacaccg

SEQ ID NO: 57 - DNA, 24 bp, synthetic construct
  source 1..24: mol_type = other DNA
caagcagaagacggcatacgagat

SEQ ID NO: 58 - DNA, 55 bp, synthetic construct
  source 1..55: mol_type = other DNA
  modified_base 51..55: mod_base = OTHER; note = phosphorothioate bonds
gtgactggagttcagacgtgtgctcttccgatctcaagttgataacggactagcc

SEQ ID NO: 59 - DNA, 20 bp, synthetic construct
  source 1..20: mol_type = other DNA
aatgatacggcgaccaccga

SEQ ID NO: 60 - DNA, 33 bp, synthetic construct; identical to SEQ ID NO: 55
  source 1..33: mol_type = other DNA

SEQ ID NO: 61 - DNA, 22 bp, synthetic construct
  source 1..22: mol_type = other DNA
  modified_base 18..22: mod_base = OTHER; note = phosphorothioate bonds
ttgtggaaaggacgaaacaccg

SEQ ID NO: 62 - DNA, 24 bp, synthetic construct
  source 1..24: mol_type = other DNA
caagcagaagacggcatacgagat

SEQ ID NO: 63 - DNA, 55 bp, synthetic construct; identical to SEQ ID NO: 58
  source 1..55: mol_type = other DNA
  modified_base 51..55: mod_base = OTHER; note = phosphorothioate bonds